SEQUENCE LISTING (53 sequences)

SEQ ID NO: 1 - DNA, 15 bp, synthetic construct
  source 1..15: mol_type = other DNA
taaagtgctgaagcc

SEQ ID NO: 2 - DNA, 16 bp, synthetic construct
  source 1..16: mol_type = other DNA
ttaaagtgcagaagcc

SEQ ID NO: 3 - DNA, 16 bp, synthetic construct
  source 1..16: mol_type = other DNA
taaagtgcagaaaatt

SEQ ID NO: 4 - RNA, 16 nt, Mus musculus
  source 1..16: mol_type = mRNA
ggcttctgcactttaa

SEQ ID NO: 5 - RNA, 32 nt, Mus musculus
  source 1..32: mol_type = mRNA
tatggggcttctgcactttaaaaaggctgtgt

SEQ ID NO: 6 - RNA, 15 nt, Homo sapiens
  source 1..15: mol_type = mRNA
ggcttcagcacttta

SEQ ID NO: 7 - RNA, 30 nt, Homo sapiens
  source 1..30: mol_type = mRNA
tgtgggcttcagcactttaaagaggctgtg

SEQ ID NO: 8 - RNA, 16 nt, Mus musculus
  source 1..16: mol_type = mRNA
aattttctgcacttta

SEQ ID NO: 9 - RNA, 34 nt, Mus musculus
  source 1..34: mol_type = mRNA
gttataattttctgcactttaatttattttacgt

SEQ ID NO: 10 - RNA, 16 nt, Homo sapiens
  source 1..16: mol_type = mRNA
aatcttctgcacttta

SEQ ID NO: 11 - RNA, 33 nt, Homo sapiens
  source 1..33: mol_type = mRNA
attgctaatcttctgcactttaatttattttat

SEQ ID NO: 12 - RNA, 16 nt, Homo sapiens
  source 1..16: mol_type = mRNA
aatactgagcacttta

SEQ ID NO: 13 - RNA, 34 nt, Homo sapiens
  source 1..34: mol_type = mRNA
gtgtaatactgagcactttacttcttaataaaaa

SEQ ID NO: 14 - DNA, 19 bp, synthetic construct
  source 1..19: mol_type = other DNA
gcccatatgtggacaggta

SEQ ID NO: 15 - DNA, 20 bp, synthetic construct
  source 1..20: mol_type = other DNA
gtgtggccagccaggaccca

SEQ ID NO: 16 - DNA, 20 bp, synthetic construct
  source 1..20: mol_type = other DNA
cgaacttatcattgtcgtac

SEQ ID NO: 17 - DNA, 20 bp, synthetic construct
  source 1..20: mol_type = other DNA
aaacttaaacccgtggttca

SEQ ID NO: 18 - DNA, 21 bp, synthetic construct
  source 1..21: mol_type = other DNA
acgtaggttccccagagagca

SEQ ID NO: 19 - DNA, 20 bp, synthetic construct
  source 1..20: mol_type = other DNA
tgtgtggccaaccaggaccc

SEQ ID NO: 20 - DNA, 20 bp, synthetic construct
  source 1..20: mol_type = other DNA
ttatatgccctgaccaccat

SEQ ID NO: 21 - DNA, 20 bp, synthetic construct
  source 1..20: mol_type = other DNA
aatgttttcggacatagaga

SEQ ID NO: 22 - DNA, 20 bp, synthetic construct
  source 1..20: mol_type = other DNA
ctaggggtcttggccattcc

SEQ ID NO: 23 - DNA, 20 bp, synthetic construct
  source 1..20: mol_type = other DNA
ctgaacctgaggacttgggg

SEQ ID NO: 24 - DNA, 20 bp, synthetic construct
  source 1..20: mol_type = other DNA
gttcctgagacaccgagagg

SEQ ID NO: 25 - DNA, 20 bp, synthetic construct
  source 1..20: mol_type = other DNA
ggcagcaaatcagcgtactt

SEQ ID NO: 26 - DNA, 20 bp, synthetic construct
  source 1..20: mol_type = other DNA
tgaggactcggggaaataaa

SEQ ID NO: 27 - DNA, 18 bp, synthetic construct
  source 1..18: mol_type = other DNA
cgtggagtcggagtggac

SEQ ID NO: 28 - DNA, 20 bp, synthetic construct
  source 1..20: mol_type = other DNA
ggtcgtgacagtgaaatcca

SEQ ID NO: 29 - DNA, 22 bp, synthetic construct
  source 1..22: mol_type = other DNA
cagaagattagcaatcggtcac

SEQ ID NO: 30 - DNA, 20 bp, synthetic construct
  source 1..20: mol_type = other DNA
tggcagcctatgctgttatg

SEQ ID NO: 31 - DNA, 21 bp, synthetic construct
  source 1..21: mol_type = other DNA
ctagacctgtcccacaaccta

SEQ ID NO: 32 - DNA, 23 bp, synthetic construct
  source 1..23: mol_type = other DNA
gcaaacacgccttcttctaatgt

SEQ ID NO: 33 - DNA, 22 bp, synthetic construct
  source 1..22: mol_type = other DNA
gcgtggtaccctcttggcagtt

SEQ ID NO: 34 - DNA, 19 bp, synthetic construct
  source 1..19: mol_type = other DNA
cacgacaatcacaacatcc

SEQ ID NO: 35 - DNA, 20 bp, synthetic construct
  source 1..20: mol_type = other DNA
catatggggcttctgcactt

SEQ ID NO: 36 - DNA, 20 bp, synthetic construct
  source 1..20: mol_type = other DNA
gaggctgggtactcacttgg

SEQ ID NO: 37 - DNA, 20 bp, synthetic construct
  source 1..20: mol_type = other DNA
attgctgccttccttacccc

SEQ ID NO: 38 - DNA, 20 bp, synthetic construct
  source 1..20: mol_type = other DNA
ctgggtactcacttggtcca

SEQ ID NO: 39 - DNA, 32 bp, synthetic construct
  source 1..32: mol_type = other DNA
cagccacgccagaccacagttgcactcaaatg

SEQ ID NO: 40 - DNA, 94 bp, Mus musculus
  source 1..94: mol_type = other DNA
aggtattgctgccttccttacctgtccacatatggggcttctgcactttaaaaaggctgtgtggccagccaggacccagggtcccctccccaca

SEQ ID NO: 41 - DNA, 41 bp, synthetic construct
  source 1..41: mol_type = other DNA
aggtattgctgccttccttaccccagggtcccctccccaca

SEQ ID NO: 42 - DNA, 38 bp, synthetic construct
  source 1..38: mol_type = other DNA
tattgctgccttccttaccccagggtcccctccccaca

SEQ ID NO: 43 - DNA, 36 bp, synthetic construct
  source 1..36: mol_type = other DNA
aggtattgctgccttcctcagggtcccctccccaca

SEQ ID NO: 44 - DNA, 40 bp, synthetic construct
  source 1..40: mol_type = other DNA
aggtattgctgccttccttacccagggtcccctccccaca

SEQ ID NO: 45 - DNA, 32 bp, synthetic construct
  source 1..32: mol_type = other DNA
gggccaggcagaacggcctccccacaggagga

SEQ ID NO: 46 - DNA, 36 bp, synthetic construct
  source 1..36: mol_type = other DNA
aggtattgctgccttccccagggtcccctccccaca

SEQ ID NO: 47 - DNA, 116 bp, synthetic construct
  source 1..116: mol_type = other DNA
gggccaggcagaacggctgcatgccacacagcctttttaaagtgcagaagccccatatgtgtaaggaaggcagcaatacctgtacccaaaagaactggcagggtcccctccccaca

SEQ ID NO: 48 - DNA, 117 bp, Mus musculus
  source 1..117: mol_type = other DNA
gtatttatataccccggctgcattatgatgctggtctttgtgaccaattgttataattttctgcactttaatttattttacgtaaacttaaacccgtggttcaaggaaggtttgtct

SEQ ID NO: 49 - DNA, 29 bp, synthetic construct
  source 1..29: mol_type = other DNA
gtaatataatgagttcagaaagaagaaaa

SEQ ID NO: 50 - DNA, 37 bp, synthetic construct
  source 1..37: mol_type = other DNA
gtgttcctgagacaccgagaggtgggagagccttcac

SEQ ID NO: 51 - DNA, 42 bp, synthetic construct
  source 1..42: mol_type = other DNA
gtgttcctgagacaccgagaggtggctgtcctcaaggaaggt

SEQ ID NO: 52 - DNA, 30 bp, synthetic construct
  source 1..30: mol_type = other DNA
gtgttcctgagacaccgagaaggtttgtct

SEQ ID NO: 53 - DNA, 107 bp, synthetic construct
  source 1..107: mol_type = other DNA
gcctaataatccacacgtgtttgtgtgttcctgagacaccgagaaggtttgtcttacttttctcatgtgagaaatctaggtgtaaatattgagttcagaaagaagaa

(19) United States
(12) Patent Application Publication
(10) Pub. No.: US 2026/0218201 A1
(43) Pub. Date: Jul. 30, 2026

(54) PKD-STABILIZING OLIGONUCLEOTIDE FOR THE TREATMENT OF AUTOSOMAL DOMINANT POLYCYSTIC KIDNEY DISEASE

(71) Applicant: THE BOARD OF REGENTS OF THE UNIVERSITY OF TEXAS SYSTEM, Austin, TX (US)

(72) Inventors: Ronak LAKHIA, Dallas, TX (US); Harini RAMALINGAM, Dallas, TX (US); Laurence BIGGERS, Dallas, TX (US); Maggie ZUMWALT, Dallas, TX (US); Patricia COBO-STARK, Dallas, TX (US); Vishal D. PATEL, Dallas, TX (US)

(73) Assignee: THE BOARD OF REGENTS OF THE UNIVERSITY OF TEXAS SYSTEM, Austin, TX (US)

(21) Appl. No.: 19/159,042

(22) PCT Filed: Feb. 23, 2024
(86) PCT No.: PCT/US2024/017086

§ 371 Date: Aug. 22, 2025

Related U.S. Application Data
(60) Provisional application No. 63/486,549, filed on Feb. 23, 2023.

(51) Int. Cl. C12N 15/113; A61P 13/12

(52) U.S. Cl. CPC C12N 15/1138; A61P 13/12; C12N 2310/11

(57) ABSTRACT

The present disclosure provides in part an antisense oligonucleotide strategy to increase levels of polycystin 1 and/or polycystin 2 (PKD1 and/or PKD2). By targeting and inhibiting regulatory elements of the 3′ UTR of PKD1 mRNA and/or PKD2 mRNA, compositions of the present disclosure are able to de-repress PKD1 and/or PKD2 mRNA and increase expression of PKD1 and/or PKD2 protein. Further methods are provided for increasing PKD1 and/or PKD2 expression in cells in vitro or in vivo as well as methods of treating autosomal dominant polycystic kidney disease using the antisense oligonucleotides provided herein.

### CROSS REFERENCE TO RELATED APPLICATION

This application claims priority to U.S. Provisional Patent Application Ser. No. 63/486,549, filed Feb. 23, 2023, and titled “PKD-STABILIZING OLIGONUCLEOTIDE FOR THE TREATMENT OF AUTOSOMAL DOMINANT POLYCYSTIC KIDNEY DISEASE,” which is incorporated by reference herein in its entirety.

### ACKNOWLEDGEMENT OF GOVERNMENT SUPPORT

This invention was made with Government support under Grant No. DK079328 awarded by the National Institutes of Health. The government has certain rights in this invention.

### INCORPORATION BY REFERENCE OF SEQUENCE LISTING

This application contains a Sequence Listing that has been submitted in XML format via Patent Center and is hereby incorporated by reference in its entirety. The CML file, created on Feb. 22, 2024, is named 106546-786464_UTSD 4017_SequenceListing.xml and is about 47,000 bytes in size.

### BACKGROUND

### 1. Field

The present disclosure relates to composition and methods for increasing polycystin protein levels to treat autosomal dominant polycystic kidney disease. More particularly, the present disclosure provides antisense oligonucleotide strategy useful for targeting PKD1 and/or PKD2 mRNA expression.

### 2. Discussion of Related Art

Autosomal dominant polycystic kidney disease (ADPKD) is a common genetic disorder, affecting approximately 12.5 million people. ~80% of ADPKD cases are caused due by mutations of the PKD1 gene. Despite the transformative potential, no direct PKD1-boosting drugs are in clinical development. ADPKD individuals inherit one mutated PKD1 allele from the affected parent, whereas the non-affected parent passes down a normal copy. A clinical hallmark of ADPKD is the relentless growth of innumerable fluid-filled cysts in the kidneys, which replace the normal parenchyma and over decades cause massive bilateral kidney enlargement and renal failure. ADPKD occurs because of heterozygous, loss-of-function mutations in PKD1 (~78% of cases) or PKD2 (~15% of cases).

New methods and therapeutics to treat this condition are needed.

### BRIEF SUMMARY

In certain aspects of the present disclosure, an antisense oligonucleotide (ASO) that hybridizes to a 3′UTR regulatory region on an mRNA encoding polycystin 1 or polycystin 2 (PKD1 or PKD2 mRNA) is provided.

In certain aspects, the ASO provided herein may interfere with a microRNA hybridizing to the 3′UTR regulatory region. In some aspects, the microRNA is microRNA-17.

In various aspects, the 3′UTR regulatory region targeted by any of the ASOs of the present disclosure can comprise a cis-inhibitory motif.

In any aspect of the present disclosure, the ASO may stabilize the PKD1 and/or PKD2 mRNA.

In any of the aspects of the present disclosure, the ASO can comprises at least 9 nucleotides. For example, in some aspects, the ASO can comprise 9 to 24 nucleotides. For example, in some aspects the ASO consists of 15 or 16 nucleotides.

In various aspects, the ASO of the present disclosure may hybridize to at least 6, at least 7, at least 8, at least 9, at least 10, at least 11, at least 12, at least 13, at least 14, at least 15, or at least 16 consecutive nucleotides of a 3′UTR regulatory region on an mRNA encoding polycystin 1 (PKD1 mRNA). In some aspects, the 3′UTR regulatory region on an mRNA encoding polycystin 1 (PKD1 mRNA) has a nucleic acid sequence of any one of SEQ ID NOs: 4 to 7.

In some aspects, the ASO of the present disclosure may hybridize to at least 6, at least 7, at least 8, at least 9, at least 10, at least 11, at least 12, at least 13, at least 14, at least 15, or at least 16 consecutive nucleotides of a 3′UTR regulatory region on an mRNA encoding polycystin 2 (PKD2 mRNA). In some aspects, the 3′UTR regulatory region on an mRNA encoding polycystin 1 (PKD2 mRNA) has a nucleic acid sequence of any one of SEQ ID NOs: 8 to 13.

In any of the foregoing or related aspects, the ASO may comprise a nucleic acid sequence having at least at least 50%, at least 60%, at least 70% or at least 80%, at least 90%, at least 95%, at least 96%, at least 97%, at least 98%, or at least 99% identity to SEQ ID NO: 1, SEQ ID NO: 2 or SEQ ID NO: 3. For example, in some aspects, the ASO has a nucleic acid sequence comprising or consisting of SEQ ID NO: 1, SEQ ID NO: 2 or SEQ ID NO: 3.

In any of the foregoing or related aspects, the ASO may comprise at least one locked-nucleic acid (LNA).

Also provided are pharmaceutical compositions comprising any of the ASOs as provided herein. In some aspects, the pharmaceutical composition further comprises a carrier or excipient.

Also provided are kits comprising any of the pharmaceutical compositions provided herein.

Further aspects of the present disclosure are directed to methods of selectively increasing expression of polycystin 1 and/or polycystin 2 in a cell, the method comprising delivering an ASO as provided herein to the cell.

In various aspects, the cell carries a mutation in at least one allele of the PKD1 and/or PKD2 gene and has reduced baseline expression of polycystin 1 and/or polycystin 2 compared to a cell without the mutation. In further aspects, delivering the ASO provided herein may increase expression of polycystin 1 and/or polycystin 2 in the cell. For example, in various aspects, expression of polycystin 1 and/or polycystin 2 may be increased by at least 25%, at least 30%, at least 40%, at least 50%, at least 60%, at least 70% or at least 80% over baseline.

In various aspects, the cell may be in vitro. In other aspects, the cell may be in vivo. In various aspects, the cell may be human or murine.

In further aspects of the present disclosure, a method of treating Autosomal Dominant Polycystic Kidney Disease (ADPKD) in a subject in need thereof is provided, the method comprising administering a pharmaceutically effective amount of an antisense oligonucleotide (ASO) as provided herein.

In various aspects, the ASO may be administered systemically (e.g., orally, intravenously, subcutaneously, or intraperitoneally).

In various aspects, the ASO may be administered as a pharmaceutical composition.

In any of the foregoing or related aspects, the subject can be human.

### BRIEF DESCRIPTION OF THE DRAWINGS

FIG. 1A-1J show how Pkd1 mRNA is cis-repressed via its 3′-UTR miR-17 binding motif. FIG. 1A depicts a graphic illustration of the CRISPR/Cas9 approach used to delete the miR-17 motif from Pkd1 3′-UTR (Pkd1^{Δ17}). FIG. 1B depicts representative immunoblots showing PKDR products obtained after amplification of tail DNA from mice with indicated genotypes. The lower band represents the Δ17 deletion. n=3 for all genotypes. FIG. 1C is a 3′-UTR nucleotide sequence of wildtype (WT) and Pkd1^{Δ17} alleles. The miR-17 binding motif and sgRNA PAM sites are highlighted in bold green and pink, respectively. The pink dashed line indicates the deleted nucleotides in Pkd1^{Δ17}. Sanger sequencing chromatogram depicting the nucleotide sequence of the Pkd1^{Δ17}. FIG. 1D depicts H&E staining, Lotus Tetragonolobus Lectin labeling (LTL, a proximal tubule marker), Tamm-Horsfall protein immunostaining (THP, a loop of Henle maker), and Dolichos Biflorus Agglutinin labeling (DBA, a collecting duct marker) showing normal kidney histology in 8-week-old Pkd1^{+/+} and Pkd1^{Δ17/Δ17} mice. FIG. 1E-1F depict normal kidney-weight-to-body-weight (KW/BW) and serum blood urea nitrogen (BUN) levels in 8-week-old Pkd1^{+/+} and Pkd1^{Δ17/Δ17} mice. FIG. 1G-1H depict images and cyst index quantification of E13.5 Pkd1^{+/+}, Pkd1^{Δ17/+}, and Pkd1^{Δ17/Δ17} kidneys grown for four days in culture media containing vehicle, 100 μM CAMP, or 100 μM CAMP plus 250 μM SAM. FIG. 11 depicts representative immunoblots depicting PKD1 expression in Pkd1^{+/+}, Pkd1^{Δ17/+} and Pkd1^{Δ17/Δ17} ex-vivo kidneys treated with vehicle, CAMP, or cAMP plus SAM. Actin is used as the loading control. FIG. 1J shows an allele-specific qRT-PKDR showing the quantity of Pkd1 mRNAs produced by the wildtype (+) and Δ17 alleles in E15.5 Pkd1^{Δ17/+} kidneys (n=5).

FIG. 2A-2J show how monoallelic Pkd1 derepression alleviates polycystic kidney disease. FIG. 2A depicts an immunoblot showing reduced PKD1 expression in Pkd1^{RC/-} compared to Pkd1^{RC/+} cells. PKD1 level was restored in Pkd1^{RCΔ17/-} cells. #1 and #2 refer to the two independent Pkd1^{RCΔ17/-} clonal cell lines. Actin serves as the loading control. n=3 biologically independent samples. FIG. 2B-2C depict representative images and quantification showing increased 3D cyst size of Pkd1^{RC/-} compared to Pkd1^{RC/+} cells cultured in Matrigel. Cyst size was normalized in Pkd1^{RCΔ17/-} cells. n=300 cysts pooled from three independent experiments. FIG. 2D shows a heatmap showing alamarBlue-assessed proliferation of Pkd1^{RC/-} and Pkd1^{RCΔ17/-} cells in the absence (−) or presence (+) of 100 μM CAMP, 17 mM glucose, or 100 μM SAM. n=8, each circle represents a biological replicate. FIG. 2E shows representative images showing Mito-tracker labeling and anti-PKDreb1 immunostaining in Pkd1^{RC/+}, Pkd1^{RC/-}, and Pkd1^{RCΔ17/-} cells. n=3 biologically dependent experiments. FIG. 2F depicts gross kidney and H&E-stained kidney sections from 18-day-old mice with the indicated genotypes. Data from the progeny of the three founders are shown separately. Founder #1: Pkd1^{RC/+} (n=7), Pkd1^{RCΔ17/+} (n=7), Pkd1^{RC/-} (n=8), and Pkd1^{RCΔ17/-} (n=7). Founder #2: Pkd1^{RC/+} (n=15), Pkd1^{RCΔ17/+} (n=19), Pkd1^{RC/-} (n=15), and Pkd1^{RCΔ17/-} (n=17). Founder #3: Pkd1^{RC/+} (n=6), Pkd1^{RCΔ17/+} (n=6), Pkd1^{RC/-} (n=8), and Pkd1^{RCΔ17/-} (n=8). FIG. 2G shows an immunoblot showing PKD1 expression in kidneys of 18-day-old mice with the indicated genotypes derived from the three founders. Actin serves as the loading control. n=3 independent kidney samples for each genotype and founder.

FIG. 2H-2I show KW/BW ratio and BUN levels in mice with the indicated genotypes. Data from all three founders are shown. Founder #1 (blue circles), Founder #2 (light pink circles), and Founder #3 (orange circles). FIG. 2J shows paired-end RNA-seq data showing the RC allele usage in Pkd1^{RC/-} (grey circles, n=5), Pkd1^{RCΔ17/-} founder #2 (pink circles, n=5), and Pkd1^{RCΔ17/-} founder #3 (orange circles, n=5). Error bars indicate SEM. Statistical analysis: One-way ANOVA, Tukey's multiple-comparisons test (c, h, i, j). Source data are provided as a Source Data file.

FIG. 3A-3D show how Pkd1 derepression attenuates cyst-pathogenic events and disease progression. FIG. 3A depicts Gross kidney images and H&E-stained kidney sections from 18-week-old mice with the indicated genotypes derived from founder #3. miR-17 motif deletion was associated with sustained benefit and suppressed long-term PKD progression. n=3 (Pkd1^{RC/+}), n=3 (Pkd1^{RCΔ17/+}), n=8 (Pkd1^{RC/-}), and n=7 (Pkd1^{RCΔ17/-}). FIG. 3B depicts KW/BW, BUN, and serum creatinine (Scr) levels in the 18-week-old progeny of founder #3.

FIG. 3C depicts a heatmap showing global mRNA expression profiles of kidneys from 18-day-old mice with the indicated genotypes. mRNAs that were dysregulated in Pkd1^{RC/-} compared to Pkd1^{RC/+} kidneys but exhibited improved expression in Pkd1^{RCΔ17/-} kidneys were chosen for visualization. #3=founder #3; #2=founder #2. n=3 (Pkd1^{RC/+}), n=3 (Pkd1^{RCΔ17/+} founder 2), n=3 (Pkd1^{RCΔ17/+} founder 3), n=5 (Pkd1^{RC/-}), n=5 (Pkd1^{RCΔ17/-} founder 2), and n=5 (Pkd1^{RCΔ17/-} founder 3). FIG. 3D depicts representative images showing phospho-Histone-H3 (pHH3), Mannose Receptor C-Type 1 (MRC1), or PKDreb1 immunostaining in kidney sections of 18-day-old and 18-week-old Pkd1^{RC/-} (n=5) and Pkd1^{RCΔ17/-} (n=5) mice. The sections were co-labeled with DBA to mark collecting duct-derived cysts. Error bars indicate SEM. Statistical analysis: One-way ANOVA, Tukey's multiple-comparisons test (b). Source data are provided as a Source Data file.

FIG. 4A-4K provide data showing Pkd2 derepression retards cyst growth in Pkd1-mutant models. FIG. 4A depicts an immunoblot showing Polycystin-2 (PKD2) expression in cells with the indicated genotypes. miR-17 motif deletion from Pkd2 3′-UTR leads to higher PKD2 expression in Pkd1^{RC/-} cells. Actin serves as the loading control. #1 and #2 refer to the two independent Pkd1^{RC/-}; Pkd2^{Δ17/Δ17} clonal cell lines. n=3 biologically independent samples for both clones. FIG. 4B-4C depict representative images and quantification showing 3D cyst size of cells with indicated genotypes grown in Matrigel cultures. n=300 cysts pooled from three independent experiments. FIG. 4D depicts representative images showing mitotracker labeling and anti-PKDreb1 immunostaining in cells with the indicated genotypes. n=3 biologically independent experiments. FIG. 4E depicts a representative heatmap showing alamarBlue-assessed proliferation of Pkd1^{RC/-} and Pkd1^{RC/-}; Pkd2^{Δ17/Δ17} cells in the absence (−) or presence (+) of CAMP, glucose, or SAM. n=8, each circle represents a biological replicate. FIG. 4F depicts H&E-stained kidney sections from 18-day-old mice with the indicated genotypes. n=3 (Pkd1^{RC/+}; Pkd2^{+/+}), n=3 (Pkd1^{RC/+}; Pkd2^{Δ17/Δ17}), n=8 Pkd1^{RC/-}; Pkd2^{+/+}), and n=11 (Pkd1^{RC/-}; Pkd2^{17/Δ17}). FIG. 4G depicts immunoblots showing PKD2, Yap1, and c-Myc expression in kidneys of 18-day-old mice with the indicated genotypes (n=3 for each group). FIG. 4H-4I depict KW/BW and serum creatinine levels in 18-day-old mice with the indicated genotypes. FIG. 4J depicts representative images showing pHH3 and MRC1 immunostaining in kidney sections from 18-day-old mice with the indicated genotypes (n=3 for each group). FIG. 4K depicts a heatmap showing differential mRNA expression in kidneys of 18-day-old mice with the indicated genotypes (n=3). mRNAs that were dysregulated in Pkd1^{RC/-} compared to Pkd1^{RC/+} kidneys but exhibited improved expression in Pkd1^{RC/-}; Pkd2^{Δ17/Δ17} kidney were chosen for heatmap visualization. Error bars indicate SEM. Statistical analysis: One-way ANOVA, Tukey's multiple-comparisons test (b, h, and i).

FIG. 5A-5N depict data showing that acute Pkd1 and Pkd2 derepression attenuates PKD. FIG. 5A-5B show qRT-PKDR and immunoblot analysis showing Pkd1/PKD1 and Pkd2/PKD2 expression in Pkd1^{RC/-} cells transfected with vehicle (PBS), 100 μM control oligonucleotide, or 100 μM RGLS4326. illustrates an aspect of the subject matter in accordance with one embodiment. FIG. 5C depicts images and quantification of 3D cyst size of Pkd1^{RC/-} cells cultured in Matrigel before (day4) or after (day7) transfection with vehicle (PBS), 100 μM control oligonucleotide, or 100 μM RGLS4326. FIG. 5D depicts H&E-stained kidney sections from 18-day-old Pkd1^{RC/-} mice injected on P10, P11, P12, and P16 either with vehicle (PBS), 20 mg/kg control oligonucleotide, or 20 mg/kg RGLS4326. H&E-stained kidney section from untreated 18-day-old wildtype mouse is shown for reference. FIG. 5E-5G show KW/BW, BUN, and serum creatinine levels in 18-day-old Pkd1^{RC/-} mice treated with vehicle (PBS), 20 mg/kg control oligonucleotide, or 20 mg/kg RGLS4326 are shown. Data from untreated 18-day-old wildtype mice are shown as a reference. FIG. 5H depicts H&E-stained kidney sections from 26-day-old Pkd1^{RC/-} mice injected on P16 and P17 with 20 mg/kg control oligonucleotide or 20 mg/kg RGLS4326. H&E-stained kidney sections from genetically matched but untreated 16-day-old Pkd1^{RC/-} mice are shown to depict disease prior to starting treatment. FIG. 5I-5K depict KW/BW, BUN, and serum creatinine levels in untreated 16-day-old or treated 26-day-old Pkd1^{RC/-} mice. FIG. 5L-5N show data from an experiment where Pkd1^{RC/-} mice were injected on P16 and P17 with vehicle, 20 mg/kg RGLS4326, or 20 mg/kg control oligonucleotide. These mice then received their respective treatment regimen every week until 18 weeks of age. The fourth cohort of Pkd1^{RC/-} mice received 20 mg/kg RGLS4326 treatment on P16, P17, and bimonthly thereafter. FIG. 5L depicts H&E-stained kidney sections of 125-day-old Pkd1^{RC/-} mice on the vehicle or RGLS4326 treatment. FIG. 5M depicts Kaplan-Meir survival curves of Pkd1^{RC/-} mice in the four treatment groups. Survival of untreated wildtype is shown as a reference. FIG. 5N depicts KW/BW ratio in mice that survived till 125 days. Wildtype mice: n=3; Pkd1^{RC/-} mice: n=2 (vehicle treatment), n=5 (weekly RGLS4326 treatment), and n=7 (bi-monthly RGLS4326 treatment). Error bars indicate SEM. Statistical analysis: One-way ANOVA, Tukey's multiple-comparisons test (a, c, e-g, i-k, and n); Mantel-Cox (m).

FIG. 6A-6H show data illustrating that PKD1^{Δ17} or PKD2^{Δ17} reduce 3D cyst growth in primary human ADPKD cultures. Specifically, CRISPR/Cas9-editing was used to delete the miR-17 motif from PKD1 3′-UTR (PKD1^{Δ17}) or PKD2 3′-UTR (PKD2^{Δ17}) in primary ADPKD cultures from four human donors (#1 through #4). FIG. 6A-6B show immunoblots showing higher PKD1 expression in PKD1^{Δ17} and higher PKD2 expression in PKD2^{Δ17} ADPKD cultures compared to their respective unedited (UE) parental ADPKD cultures. Protein bands are 460 kDa (FIG. 6A) and 110-120 kDa (FIG. 6B). Actin serves as loading control. FIG. 6C-6F show images and quantification showing reduced cyst size of PKD1^{Δ17} and PKD2^{Δ17} compared to their respective unedited (UE) parental ADPKD cultures. FIG. 6G-6H show images showing higher mitotracker labeling (red) and reduced PKDREB1 immunostaining (green) in PKD1^{Δ17} and PKD2^{Δ17} ADPKD cultures compared to their respective unedited parental ADPKD cultures. n=3 biologically independent experiments for each cell line. Errors bars represent SEM, Statistical analysis: Two-tailed Students t-test (e-f). Source data are provided as a Source Data file.

FIG. 7A depicts H&E-stained kidney sections of 6-week-old Pkd1^{+/+} and Pkd1^{Δ17/Δ17} mice.

FIG. 7B-7C show Kidney-weight-to-body-weight (KW/BW) ratio and BUN levels of 6-week-old Pkd1^{+/+} and Pkd1^{Δ17/Δ17} mice. Error bars indicate SEM. Statistical analysis two-tailed Student's t-test.

FIG. 7D depicts an immunoblot showing PKD1 expression in Pkd1^{+/+}, Pkd1^{-/-} cell lines and in kidneys of 6 or 18-week-old Pkd1++ and Pkd1^{Δ17/Δ17} mice. (n=5 for each genotype). Lysates from Pkd1^{-/-} cells serve as negative control and do not exhibit PKD1 expression.

FIG. 8A-8B show data for the validation of the 7E12 PKD1 antibody by testing in Pkd1^{+/+} and Pkd1^{-/-} collecting duct cell lines. FIG. 8A shows qRT-PKDR showing that the Pkd1/cells do not express the Pkd1 mRNA. FIG. 8B shows an immunoblot showing absence of full-length PKD1 protein in the Pkd1-cell line. n=3 biologically independent samples from the indicated cell lines.

FIG. 9 shows data showing that miR-17 family expression declines with postnatal kidney maturation. Microarray signal intensity values for miRNAs belonging to the miR-17 family, miR-17, miR-20a, miR-20b, miR-106a, miR-106b, or miR-93 in mouse kidneys at ages P2, P7, P14, P21, and P35. The miR-17 family members show an age-dependent decrease in expression. n=4 mouse kidneys at P2, n=3 mouse kidneys at P7, P14, P21 or P35. Error bars indicate SEM. Statistical analysis one-way ANOVA, test for linear trend.

FIG. 10A-10C shows data showing that Pkd1 is cis-inhibited via its miR-17 3′-UTR motif. Allele-specific qRT-PKDR analysis showing the quantity of Pkd1 mRNAs produced by the wildtype (+) and 417 alleles in ex vivo kidney cultures of Pkd1^{Δ17/+} mice treated with vehicle (FIG. 10A), c-AMP (FIG. 10B), or c-AMP plus SAM (FIG. 10C). The Pkd1^{Δ17} allele produced more mRNA transcripts compared to the Pkd1⁺ allele. This difference was even more pronounced in the presence of c-AMP. n=5 ex vivo kidney cultures. Statistical analysis: paired t-test. Error bars indicate SEM.

FIG. 11A-11B relate to the characterization of CRISPR-edited Pkd1^{RC/-} cell lines. FIG. 11A shows PKDR products obtained after amplifying the DNA (encoding the Pkd1 3′UTR segment) from parental and CRISPR-edited cell lines. The lower band indicates the Δ17/− genotype. FIG. 11B shows graphical illustration of Sanger sequencing results from the Δ17/− bands of each clone confirming deletion of the miR-17 motif from both Pkd1 alleles.

FIG. 12A-12C relate to the phenotypic characterization of CRISPR-edited Pkd1^{RCΔ17} cell lines. FIG. 12A shows Alamarblue assay showing reduced proliferation of Pkd1 clones which lack miR-17 motif compared to Pkd1^{RC/-} parent cell line at 12 hours. n=9 biologically independent experiments. FIG. 12B shows Mitotracker images and IF staining for PKDreb1 showing restored mitochondrial membrane potential (red) and reduced PKDreb1 (green) expression in Pkd1^{RCΔ17/-} clone #2 compared to the parental cell line. n=3 biologically independent experiments. FIG. 12C shows western blot characterization of both Pkd1^{RCΔ17/-} cell lines showing reduced expression of cyst promoting genes Yap1, PKDreb1, and c-Myc compared to parental Pkd1^{RC/-} cells. As a pertinent control, PKD2 expression remained unchanged. Actin serves as the loading control. n=3 biologically independent experiments. Error bars indicate SEM. Statistical analysis: one-way ANOVA, Tukey's multiple comparisons test (a).

FIG. 13A-13B relate to PKD1 derepression in CRISPR-edited Pkd1^{RCΔ17/-}? cell lines and mouse kidneys wherein two independent PKD1 antibodies (7E12 from Santa Cruz and E8-8C3C10 from U Maryland) were used to examine PKD1 expression in Pkd1^{RCΔ17/-}? cell lines and kidneys. The 7E12 antibody detects the full-length 462 kDa PKD1, whereas the E8-8C3C10 antibody detects the 140 kDa c-terminal fragment (CTF) of the PKD1 protein. FIG. 13A shows an illustrative immunoblot showing full-length PKD1 and PKD1-CTF expression in the Pkd1^{RCΔ17/-} clones #1 and #2 compared to the control Pkd1^{RC/+} and Pkd1^{RC/-}. n=3 biologically independent experiments. FIG. 13B shows immunoblots using the E8 antibody showing PKD1 expression in the kidneys of mice with the indicated genotypes. Analysis of progeny from all three founders (#1, #2, and #3) is shown. PKD1 western blots using the 7E12 antibody are shown in FIG. 2. Both antibodies exhibit consistent results in cells and kidney tissue. Actin is used as the loading control. n=3 biologically independent kidney samples for each genotype.

FIG. 14 relates to the characterization of CRISPR-edited Pkd1^{RC/RC} mice and depicts a graphical illustration of Sanger sequencing from tail DNA from the three CRISPR-edited founders. Founders #1 and #2 harbor 108 bp and 53 base pair deletions, respectively, including the miR-17 motif. Founder #3 also lacked the miR-17 motif but acquired a 72 base pair insertion (blue), resulting in a net loss of 18 base pairs in the 3′-UTR sequence.

FIG. 15A-15F present data showing that monoallelic Pkd1 derepression suppresses disease progression, wherein a cohort of progeny derived from founder #2 was prospectively monitored till eight weeks of age. FIG. 15A shows H&E-stained kidney sections of mice of the indicated genotypes that survived till eight weeks of age are shown. Pkd1^{RC/+} (n=3), Pkd1^{RCΔ17/+} (n=3), Pkd1^{RC/-} (n=4), and Pkd1^{RCΔ17/-} (n=16). FIG. 15B shows Kaplan-Meir survival curves of mice with the indicated genotypes are shown. FIG. 15C-15E show KW/BW, serum creatinine (Scr), and BUN levels of the surviving 8-week-old mice with indicated genotypes. FIG. 15F shows immunoblots of Yap1 and c-Myc expression in kidneys of 18-day-old mice with the indicated genotypes (n=3 for all genotypes). Actin serves as the loading control. Error bars indicate SEM. Statistical analysis: one-way ANOVA, Tukey's multiple comparisons test (c-e); Log-rank Mantel-Cox (b).

FIG. 16A-16B relate to the characterization of Pkd1^{RC/-} cell lines lacking miR-17 motif from Pkd2 3′-UTR. FIG. 16A depicts PKDR products obtained after amplifying the DNA (encoding the Pkd2 3′UTR segment) from parental and CRISPR-edited cell lines. The lower bands indicate the Δ17 genotype. FIG. 16B shows a graphical illustration of Sanger sequencing results from the Δ17/− bands of each clone confirm deletion of the miR-17 motif from both Pkd2 alleles.

FIG. 17A-17C relate to the phenotypic characterization of CRISPR-edited Pkd1^{RC/-}; Pkd2^{Δ17/Δ17}? cell lines. FIG. 17A shows qRT-PKDR showing Pkd2 derepression in both Pkd1^{RC/-}; Pkd2^{Δ17/Δ17}?clones #1 and #2 compared to the parental Pkd1^{RC/-} cell line. n=3 biologically independent samples from the indicated cell lines. FIG. 17B shows Western blot characterization shows a reduced expression of cyst-promoting genes Yap1, Mettl3, c-Myc, and PKDreb1 in both Pkd1^{RC/-}; Pkd2^{Δ17/Δ17}? clones compared to the parent Pkd1^{RC/-} cell line. Pertinently, PKD1 expression remained unchanged. Actin serves as a loading control. n=3 biologically independent samples from the indicated cell lines. FIG. 17C shows Mitotracker images and IF staining for PKDreb1 showing restored mitochondrial membrane potential (red) and reduced PKDreb1 (green) expression, respectively, in the Pkd1^{RC/-}; Pkd2^{Δ17/Δ17}?clone #2 compared to the parental cell line. n=3 biologically independent samples from the indicated cell lines.

FIG. 18 relates to the CRISPR-editing and characterization of Pkd2417? mice that lack miR-17 motif in Pkd2 3′-UTR (see FIG. 1A) and shows a graphical illustration and Sanger sequencing results of PKDR product from the tail DNA of founder mouse showing 139 base pair deletion from the Pkd2 3′-UTR, including the miR-17 motif.

FIG. 19A-19E relate to the phenotypic characterization of RGLS4326 treatment on Pkd1^{RC/-} cells wherein Pkd1^{RC/-} cells were transfected with 100 μM RGLS4326, 100 μM control oligonucleotide, or vehicle control. Seventy-two hours later, cells were equivalently seeded in 96 well plate for 12 hours for the alamarBlue assay or placed in matrigel for seven days for a 3D cyst assay. FIG. 19A shows reduced proliferation of Pkd1^{RC/-} cells treated with RGLS4326 compared to cells treated with control oligonucleotide or vehicle control. n=9 biologically independent experiments for each treatment group. FIG. 19B-19C show representative images and quantification show a reduction in cyst size of RGLS4326-treated cells compared to vehicle or control oligonucleotide-treated Pkd1^{RC/-} cells. No significant change was observed in cyst size between vehicle control and control oligonucleotide-treated groups. FIG. 19D depicts immunoblots showing Yap1, c-Myc, and PKDreb1 expression in Pkd1^{RC/-} transfected with a vehicle, control oligonucleotide or RGLS4326. Actin is the loading control. n=3 biologically independent experiments for each treatment group. FIG. 19E shows mitotracker labeling and anti-PKDreb1 immunostaining of Pkd1^{RC/-} cells transfected with a vehicle, control oligonucleotide, or RGLS4326. n=3 biologically independent experiments for each treatment group. Error bars indicate SEM. Statistical analysis: one-way ANOVA, Tukeys' multiple comparisons test (a,c).

FIG. 20A-20G relate to the characterization of RGLS4326 treatment on CRISPR-edited Pkd1^{RCΔ17/-}? or Pkd1^{RC/-}; Pkd2^{Δ17/Δ17?}? cell lines. FIG. 20A-20C depict Pkd1^{RC/-} (FIG. 20A), Pkd1^{RCΔ17/-} clone #1 (FIG. 20B), and Pkd1^{RCΔ17/-} clone #2 (FIG. 20C) cells were transfected with 100 μM RGLS4326 or vehicle control. Seventy-two hours later, protein from these cells was analyzed by western blot to assess PKD1 and PKD2 expression. FIG. 20A: Expression of PKD1 and PKD2 was increased in the Pkd1^{RC/-} cells upon RGLS4326 treatment. FIG. 20B-20C: No additional PKD1 upregulation was observed after RGLS4326 treatment in the Pkd1^{RCΔ17/-} clones #1 or #2, indicating that this oligo medicates PKD1 derepression via the miR-17 motif in the Pkd1 3′-UTR. As expected, PKD2 expression was increased in the Pkd1^{RCΔ17/-} clones #1 and #2 with RGLS4326 treatment. Actin serves as the loading control. Quantification of the western blots is shown in the graphs below (n=3 biologically independent samples from each of the indicated cell lines and treatment groups).

FIG. 20D-20E show representative images and quantification showing cyst size of vehicle or 100 μM RGLS4326-treated Pkd1^{RC/-}, Pkd1^{RCΔ17/-} (clone #1), or Pkd1^{RCΔ17/-} (clone #2). FIG. 20F-20G show representative images and quantification showing cyst size of vehicle or 100 μM RGLS4326-treated Pkd1^{RC/-}, Pkd1^{RC/-}; Pkd2^{Δ17/Δ17} (clone #1) or Pkd1^{RC/-}; Pkd2417/Δ17 (clone #2). Error bars indicate SEM. Statistical analysis: Two-tailed Student's T-test (a-c); one-way ANOVA, Tukeys' multiple comparisons test (e and g).

FIG. 21A-21C relate to the characterization of long-term RGLS4326 treatment in Pkd1^{RC/-} mice. FIG. 21A shows low and high magnification images of H&E-stained kidneys from P125-day-old Pkd1^{RC/-} mice treated with vehicle or RGLS4326 are shown. The high magnification images are from the regions marked by black insets on the low magnification images. Substantially preserved histology was observed in kidneys of Pkd1^{RC/-} mice treated with RGLS4326 compared to vehicle. FIG. 21B shows BUN levels of Pkd1^{RC/-} mice treated with vehicle (grey, n=1) or RGLS4326 (shades of blue, n=6). BUN from age-matched, non-cystic controls (brown, n=4) is shown as a reference of the normal values. The comparison is limited because the blood from only one of the two surviving vehicle-treated Pkd1^{RC/-} mice was available for renal function measurement. BUN levels are not available for the control oligo-treated Pkd1^{RC/-} mice because none of them survived till the end of the study at 125 days. FIG. 21C shows IF staining showing reduced expression of fibrosis markers, α-SMA and Vimentin in kidneys from Pkd1^{RC/-} mice treated with RGLS4326 compared to vehicle (n=2 for vehicle, n=3 for each treatment group). Statistical analysis: ANOVA, Tukeys' multiple comparisons test (b).

FIG. 22A-22D relate to genotyping of CRISPR-edited primary human ADPKD cultures and. FIG. 22A-22B show PKDR products obtained after amplifying the DNA encoding the PKD1 (FIG. 22A) or PKD2 (FIG. 22B) 3′UTR segment from unedited parental and CRISPR-edited human ADPKD cultures. The arrows indicate the PKDR bands resulting from miR17 motif deletion in PKD1 (FIG. 22A) and PKD2 (FIG. 22B) genes. FIG. 22C-22D show Sanger sequencing of the PKDR product (black rectangles) aligned with the human genome (purple rectangles). The deleted region contains the miR-17 binding site (green rectangles).

FIG. 23A-23B depict data showing reduced proliferation in PKD1^{Δ17} and PKD2^{Δ17} edited ADPKD cultures and show alamarBlue-assessed proliferation of ADPKD donor cultures that were CRISPR-edited to remove the miR-17 motif in either the PKD1 (pink, FIG. 23A) or PKD2 (Green, FIG. 23B) gene compared to their respective unedited (UE) parental controls (grey). Error bars indicate SEM. n's indicate biologically independent experiments for each cell line and treatment. Statistical test: Two-tailed Students t-test for each cell line separately.

FIG. 24A is a graphical description of PKD1 stabilizing oligo binding to 3′ untranslated region (UTR) of PKD1 mRNA preventing miR-17 engagement.

FIG. 24B is an alignment of PKD1 3′UTR and conservation track with annotation of miR-17 seed (grey) and localization of PKD1 oligo targeting region (green).

FIG. 24C is a plot showing luminescence activity measured 72 hours after transfection in IMCD3 cells transfected with pls-PKD1-3′-UTR reporter plasmid, microRNA mimic (sc or miR-17) and scramble (ctl) or Pkd1 oligo. Luminescence activity measured 72 hours after transfection demonstrates increased signal in cells treated with PKD1 oligo.

FIG. 24D is a plot of results from a qRT-PKDR for Pkd1 mRNA spanning exon 4-5 in scramble and Pkd1 oligo treated mouse kidney epithelial cells.

FIG. 24E is a plot showing Pkd1 oligo binding prevents cDNA synthesis over Pkd1-3′-UTR binding site evidenced by lack of Pkd1 3′-UTR transcript detection in Pkd1 oligo treated cells

FIG. 24F is an illustrative immunoblot showing increased Polycystin 1 (PKD1) in Pkd1 oligo treated kidney epithelial cells.

FIG. 25A depicts luminescence activity measured from Pkd1^{RC/-} cells transfected with Pls-PKD1-3′-UTR reporter plasmid and Pkd1 or scramble oligo. Luminescence activity measured after 72 hours shows increased Pkd1-3′-UTR activity in Pkd1 oligo treated cells.

FIG. 25B depicts levels of Pkd1 mRNA transcript spanning exon 4-5 (left) or Pkd1 3′-UTR transcript (right) in Pkd1^{RC/-} cells transfected with Pkd1 or scramble oligo.

FIG. 25C-25D depict plots of abundance of Pkd1 (FIG. 25C) and c-Myc (FIG. 25D) mRNA transcripts in Pkd1^{RC/-} cells transfected with scramble or Pkd1 oligo and treated with actinomycin to inhibit transcription 48 hours after transfection. Samples were harvested for at 0, 4 and 8 hours to measure abundance of mRNA transcripts. Pkd1 mRNA degradation is inhibited by Pkd1 oligo. c-Myc transcript is equivalently degraded in both scramble and Pkd1 oligo treated samples.

FIG. 25E-25F depicts representative fluorescent images (FIG. 25F) and quantification (FIG. 25E) of Pkd1 mRNA abundance in cells transfected with scramble (Sc) or a pDAC565 plasmid bearing a guide RNA targeting exon 4 of the Pkd1 gene (Pkd1^{RC/-}).

FIG. 25G-25H depicts representative fluorescent images (FIG. 25G) and quantification (FIG. 25H) of Pkd1 mRNA abundance in cells transfected with scramble (Sc) or Pkd1 oligo and dDAC565 plasmid bearing a guide RNA targeting exon 4 of the Pkd1 gene (Pkd1 oligo).

FIG. 25I depicts a western blot and quantification showing increased Polycystin 1 protein (PKD-1) expression in Pkd1 oligo treated Pkd1^{RC/-} cells (pink) compared to Scramble treated cells (purple).

FIG. 25J shows phase contrast images and quantification showing reduced cyst size in Pkd1 oligo treated Pkd1^{RC/-} cells (pink) compared to Scramble treated cells (purple).

FIG. 25K depicts representative fluorescent images of Pkd1 oligo treated cells showing increased mitochondrial activity measured by MitoTracker signal (red) and reduced PKDREB (green) expression.

FIG. 26A depicts representative immunoblots showing increased PKD1 expression of the remaining PKD1 allele in three immortalized human ADPKD kidney epithelial cell lines treated with Pkd1 oligo.

FIG. 26B depicts phenotypic and molecular analysis of 2 immortalized human ADPKD kidney epithelial cell lines treated with Pkd1 oligo showing reduced cyst size, increased MitoTracker signal (red) and reduced PKDREB (green) expression.

FIG. 27A shows from left to right, plots depicting levels of Pkd1 full transcript (far left), Pkd1 3-UTR transcript (middle) and a representative immunoplot showing Polycystin 1 (PC1) expression (far right), in murine Pkd1^{RC/-} cells treated with scrambled (SC), Pkd1 oligo #1 (1) or Pkd1 oligo #2 (2).

FIG. 27B shows from left to right, plots depicting levels of Pkd1 full transcript (far left), Pkd1 3-UTR transcript (middle) and a representative immunoplot showing Polycystin 1 (PC1) expression (far right), in a human ADPKD cell line (Donor 3) treated with scrambled (SC), Pkd1 oligo #1 (1) or Pkd1 oligo #2 (2).

FIG. 27C shows from left to right, plots depicting levels of Pkd1 full transcript (far left), Pkd1 3-UTR transcript (middle) and a representative immunoplot showing Polycystin 1 (PC1) expression (far right), in a human ADPKD cell line (Donor 4) treated with scrambled (SC), Pkd1 oligo #1 (1) or Pkd1 oligo #2 (2).

FIG. 27D shows from left to right, plots depicting levels of Pkd1 full transcript (far left), Pkd1 3-UTR transcript (middle) and a representative immunoplot showing Polycystin 1 (PC1) expression (far right), in a human ADPKD cell line (WT9-7) treated with scrambled (SC), Pkd1 oligo #1 (1) or Pkd1 oligo #2 (2).

FIG. 28 shows a plot depicting PKD2 expression in mIMCD3 cells treated with scrambled miR (“sc”) or miR-17 (“17”) alongside a scrambled oligo (“sc”) or a Pkd2 oligo (“Pkd2”, SEQ ID NO: 3).

### DETAILED DESCRIPTION

The following detailed description references the accompanying drawings that illustrate various aspects of the present disclosure. The drawings and description are intended to describe aspects and aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

The present disclosure is based, at least in part, on the discovery of compositions and methods for treating autosomal dominant polycystic kidney disease through targeted modulation of polycystin 1 (PKD1) and/or polycystin 2 (PKD2) protein expression using 3′UTR-masking compositions. The disclosure shows antisense oligonucleotides (ASOs) targeting the 3′UTRs of PKD1 mRNA and/or PKD2 mRNA are useful in modulating protein expression by, in part, blocking repressive sites (e.g., cis inhibitory sites targeted by certain microRNAs like miR-17). Specifically, the present disclosure is related to the surprising discovery that eliminating a cis-inhibitory (microRNA-17 binding) motif from the 3′-UTR of mRNA expressed from a non-mutated PKD1 allele copy is sufficient to improve PKD1 dosage and ameliorate PKD1 deficiency in mice. To this end, the Applicant has designed antisense oligonucleotides to selectively increase levels of PKD1 and/or PKD2 by targeting the cis-inhibitory motif of its mRNA. Further aspects of the present disclosure also provide therapeutic strategies to selectively increase PKD1 and/or PKD2 protein expression for subjects in need thereof.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims. All publications, patent applications, patents and other references mentioned herein are incorporated by reference in their entirety.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A non-limiting example of stringent hybridization conditions are hybridization in 6× sodium chloride/sodium citrate (SSC) at about 45° C., followed by one or more washes in 0.2×SSC, 0.1% SDS at 50-65° C. (e.g., 50° C. or 60° C. or 65° C.). Preferably, the isolated nucleic acid molecule of the invention that hybridizes under stringent conditions corresponds to a naturally occurring nucleic acid molecule. As used herein, a “naturally-occurring” nucleic acid molecule refers to an RNA or DNA molecule having a nucleotide sequence that occurs in a human cell in nature (e.g., encodes a natural protein).

As used herein, the term “nucleic acid molecule” is intended to include DNA molecules (e.g., cDNA or genomic DNA) and RNA molecules (e.g., mRNA or miRNA) and analogs of the DNA or RNA generated using nucleotide analogs. The nucleic acid molecule may be single-stranded or double-stranded.

An “isolated nucleic acid molecule” means that the material is removed from its original environment (e.g., the natural environment if it is naturally occurring). For example, a naturally occurring polynucleotide present in a living animal is not isolated, but the same polynucleotide or polypeptide, separated from some or all of the coexisting materials in the natural system, is isolated, even if subsequently reintroduced into the natural system. Such polynucleotides may be part of a vector or other composition and still be isolated in that such vector or composition is not part of its natural environment.

A “nucleic acid vector” is a nucleic acid sequence designed to be propagated and or transcribed upon exposure to a cellular environment, such as a cell lysate or a whole cell. A “gene therapy vector” refers to a nucleic acid vector that also carries functional aspects for transfection into whole cells, with the intent of increasing expression of one or more genes or proteins. In each case, such vectors usually contain a “vector propagation sequence” which is commonly an origin of replication recognized by the cell to permit the propagation of the vector inside the cell. A wide range of nucleic acid vectors and gene therapy vectors are familiar to those skilled in the art.

A miRNA is a small non-coding RNA molecule which functions in transcriptional and post-transcriptional regulation of gene expression. A miRNA functions via base-pairing with complementary sequences within mRNA molecules, usually resulting in gene silencing via translational repression or target degradation. A mature miRNA is processed through a series of steps from a larger primary RNA transcript (pri-miRNA), or from an intron comprising a miRNA (mirtron), to generate a stem loop pre-miRNA structure comprising the miRNA sequence. A pre-miRNA is then cleaved to generate the mature miRNA.

Primary miRNA transcripts are transcribed by RNA polymerase II and may range in size from hundreds to thousands of nucleotides in length (pri-mRNA). Pri-miRNAs may encode for a single miRNA but may also contain clusters of several miRNAs. The pri-miRNA is subsequently processed into an about 70 nucleotide hairpin (pre-miRNA) by the nuclear ribonuclease III (RNase III) endonuclease, Drosha. Thus, isolated nucleic acid molecules of the invention have various preferred lengths, depending on their intended targets. When targeted to pri-miRNA, preferred lengths vary between 100 and 200 nucleotides, e.g., 100, 120, 150, 180 or 200 nucleotides. In the cytoplasm, a second RNAse III, Dicer, together with its dsRBD protein partner, cuts the pre-miRNA in the stem region of the hairpin thereby liberating an about 21 nucleotide RNA-duplex. Thus, isolated polynucleotides of about 80, 70, 60, 50, 40, 30, 25, 21, 20, 19, 18, 17, 16, 15, 14, 13, 12, 11, 10, 9, 8, 7, or 6 nucleotides in length are also considered in one embodiment of the invention.

As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95%, or 98% identity are defined herein as sufficiently identical.

The term “sample” refers to a cell, a population of cells, biological samples, and subjects, such as mammalian subjects. The term “biological sample” refers to tissues, cells and biological fluids isolated from a subject, as well as tissues, cells and fluids present within a subject.

As used herein, “subject” refers to a living organism having a central nervous system. In particular, subjects may include, but are not limited to, human subjects or patients and companion animals. Exemplary companion animals may include domesticated mammals (e.g., dogs, cats, horses), mammals with significant commercial value (e.g., dairy cows, beef cattle, sporting animals), mammals with significant scientific value (e.g., captive or free specimens of endangered species), or mammals which otherwise have value. Suitable subjects may also include mice, rats, dogs, cats, ungulates such as cattle, swine, sheep, horses, and goats, lagomorphs such as rabbits and hares, other rodents, and primates such as monkeys, chimps, and apes. In some embodiments, subjects may be diagnosed with autosomal dominant polycystic kidney disease or may be at risk for autosomal dominant polycystic kidney disease. Subjects may be of any age including newborn, adolescent, adult, middle age, or elderly.

As used herein, a “pharmaceutical composition” includes a pharmacologically effective amount of a therapeutic agent of the invention and a pharmaceutically acceptable carrier. As used herein, “pharmacologically effective amount,” “therapeutically effective amount” or simply “effective amount” refers to that amount of an agent effective to produce the intended pharmacological, therapeutic or preventive result. For example, if a given clinical treatment is considered effective when there is at least a 15% reduction in a measurable parameter associated with a disease or disorder, a therapeutically effective amount of an agent for the treatment of that disorder or disease is the amount necessary to effect at least a 15% reduction in that parameter.

The term “pharmaceutically acceptable carrier” refers to a carrier for administration of a therapeutic agent. Such carriers may include, but are not limited to, saline, buffered saline, dextrose, water, glycerol, ethanol, and combinations thereof. The term specifically excludes cell culture medium. For drugs administered orally, pharmaceutically acceptable carriers may include, but are not limited to, pharmaceutically acceptable excipients such as inert diluents, disintegrating agents, binding agents, lubricating agents, sweetening agents, flavoring agents, coloring agents and preservatives. Suitable inert diluents may include sodium and calcium carbonate, sodium and calcium phosphate, and lactose, while corn starch and alginic acid are suitable disintegrating agents. Binding agents may include starch and gelatin, while the lubricating agent, if present, may generally be magnesium stearate, stearic acid or talc. If desired, the tablets may be coated with a material such as glyceryl monostearate or glyceryl distearate to delay absorption in the gastrointestinal tract.

As used herein, “percent complementarity” means the percentage of nucleotides of a modified oligonucleotide that are complementary to a microRNA. Percent complementarity may be calculated by dividing the number of nucleotides of the modified oligonucleotide that are complementary to nucleotides at corresponding positions in the microRNA by the total length of the modified oligonucleotide.

As used herein, “oligonucleotide” means a polymer of linked nucleosides, each of which may be modified or unmodified, independent from one another.

As used herein, “anti-miR” means an oligonucleotide having a nucleotides sequence complementary to a microRNA. In certain embodiments, an anti-miR is a modified oligonucleotide.

As used herein, “internucleoside linkage” means a covalent linkage between adjacent nucleosides.

As used herein, “linked nucleosides” means nucleosides joined by a covalent linkage.

As used herein, “nucleobase” means a heterocyclic moiety capable of non-covalently pairing with another nucleobase.

As used herein, “nucleoside” means a nucleobase linked to a sugar.

As used herein, “nucleotide” means a nucleoside having a phosphate group or other internucleoside linkage forming group covalently linked to the sugar portion of a nucleoside.

As used herein, “modified oligonucleotide” means an oligonucleotide having one or more modifications relative to a naturally occurring terminus, sugar, nucleobase, and/or internucleoside linkage.

As used herein, “modified internucleoside linkage” means any change from a naturally occurring internucleoside linkage.

As used herein, “phosphorothioate internucleoside linkage” means a linkage between nucleosides where one of the non-bridging atoms is a sulfur atom.

As used herein, “modified sugar” means substitution and/or any change from a natural sugar.

As used herein, “modified nucleobase” means any substitution and/or change from a natural nucleobase.

As used herein, “5-methylcytosine” means a cytosine modified with a methyl group attached to the 5′ position.

As used herein, “2′fluoro sugar” means a sugar having a fluorine modification at the 2′ position.

As used herein, “2′-O-methyl sugar” or “2′-OMe sugar” means a sugar having an O-methyl modification at the 2′ position.

As used herein, “2′-O-methoxyethyl sugar” or “2′-MOE sugar” means a sugar having an O-methoxyethyl modification at the 2′ position.

As used herein, “2′-O-fluoro” or “2′-F” means a sugar having a fluoro modification at the 2′ position.

As used herein, “bicyclic sugar moiety” means a sugar modified by the bridging of two non-geminal ring atoms.

As used herein, “locked nucleic acid (LNA)” means a nucleic acid comprising one or more nucleosides having a substituted sugar moiety having a (CH₂)—O bridge between the 4′ and 2′ furanose ring atoms.

In practicing the present invention, many conventional techniques in molecular biology, microbiology, and recombinant DNA may be used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins eds.); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian cells, 1987 (J. H. Miller and M. P. Calos eds., Cold Spring Harbor Laboratory); and Methods in Enzymology, Vol. 154 and Vol. 155 (Wu and Grossman, and Wu, eds., respectively).

### II. Compositions

Various aspects of the present disclosure are directed to antisense oligonucleotides (ASO) that specifically target regulatory elements on mRNA expressed from the PKD1 gene (hereinafter referred to as PKD1 mRNA). Further aspects of the present disclosure are directed to antisense oligonucleotides (ASO) that specifically target regulatory elements on mRNA expressed from the PKD2 gene (hereinafter referred to as PKD2 mRNA). These ASOs interfere with binding of microRNA elements to 3′UTR regions of the mRNA and, in this way, extend the lifetime of the mRNA. These ASOs can restore polycystin 1 (PKD1) and/or polycystin 2 (PKD2) expression in cells or subjects that do not normally express sufficient levels of either or both proteins, and thereby provide a means to treat autosomal dominant polycystic kidney disease (ADPKD).

Accordingly, in various aspects, an antisense oligonucleotide is provided that selectively hybridizes (e.g., under stringent conditions) to a 3′UTR regulatory region on a PKD1 mRNA and/or PKD2 mRNA. By binding to this region, the ASO interferes with a microRNA (e.g., microRNA-17) hybridizing to the same region and therefore stabilizes the mRNA. As used herein, the term “stabilizes” refers to increasing the longevity and/or preventing degradation of the mRNA. An mRNA that is “stabilized” herein would be able to be translated more frequently and therefore would result in a higher titer of the encoded protein.

As mentioned, the ASOs provided herein selectively hybridize to a 3′UTR regulatory region of the PKD1 mRNA or PKD2 mRNA (e.g., a human or murine PKD1 or PKD2 mRNA). Illustrative regions of Pkd1 and Pkd2 3′ UTR in both human and mice mRNA are shown in Table 1 below. An antisense oligonucleotide of the present disclosure typically comprises a region of nucleotide sequence that hybridizes under stringent conditions to at least about 6, 7, 8, 9, 10, 11, 12, 13, 14, 15, 16, or more consecutive nucleotides of the sense or antisense sequence of 3′UTR sequence of PKD1 mRNA or PKD2 mRNA as described in Table 1 below.

TABLE 1
PKD 3′ UTRs

| | | | SEQ |
|---|---|---|---|
| PKD mRNA | | | ID |
| 3′UTR* | Species | Sequence | NO: |
| Pkd1 | Mouse | GGCTTCTGCACTTTAA | 4 |
| Pkd1 | Mouse | TATGGGGCTTCTGCACT | 5 |
| (expanded) | | TTAAAAAGGCTGTGT | |
| Pkd1 | Human | GGCTTCAGCACTTTA | 6 |
| Pkd1 | Human | TGTGGGCTTCAGCACTT | 7 |
| (expanded) | | TAAAGAGGCTGTG | |
| Pkd2 | Mouse | AATTTTCTGCACTTTA | 8 |
| Pkd2 | Mouse | GTTATAATTTTCTGCAC | 9 |
| (expanded) | | TTTAATTTATTTTACGT | |
| Pkd2 | Human | AATCTTCTGCACTTTA | 10 |
| Pkd2(expanded) | Human | ATTGCTAATCTTCTGCA | 11 |
| | | CTTTAATTTATTTTAT | |
| Pkd2 site 2 | Human | AATACTGAGCACTTTA | 12 |
| Pkd2 site 2 | Human | GTGTAATACTGAGCACT | 13 |
| (expanded) | | TTACTTCTTAATAAAAA | |

*Expanded sequences include an exemplary minimum sequence targeted by the ASOs described herein (bolded) and also include additional nucleotides at the 5′ and 3′ ends to allow for target flexibility.

In some aspects, the ASOs selectively hybridize to a 3′UTR regulatory region of the PKD1 mRNA. The 3′UTR regulatory region of the PKD1 mRNA can comprise any one of SEQ ID NOs 4-7. In some aspects, the ASOs of the present disclosure hybridizes under stringent conditions to at least about 6, 7, 8, 9, 10, 11, 12, 13, 14, 15, 16, or more consecutive nucleotides of the sense or antisense sequence of 3′UTR sequence of PKD1 mRNA provided as SEQ ID NOs: 5 or 7. For example, in some aspects the ASOs of the present disclosure hybridize under stringent conditions to at least about 6, 7, 8, 9, 10, 11, 12, 13, 14, 15, 16, or more consecutive nucleotides of the sense or antisense sequence of 3′UTR sequence of PKD1 mRNA provided as SEQ ID NOs: 4 or 6. As a further example, in some aspects the ASOs of the present disclosure hybridize under stringent conditions to the sense or antisense sequence of 3′UTR sequence of PKD1 mRNA provided as SEQ ID NO: 4 or 6.

In some aspects, the ASOs selectively hybridize to a 3′UTR regulatory region of the PKD2 mRNA. The 3′UTR regulatory region of the PKD2 mRNA can comprise any one of SEQ ID NOs 8-13. In some aspects, the ASOs of the present disclosure hybridizes under stringent conditions to at least about 6, 7, 8, 9, 10, 11, 12, 13, 14, 15, 16, or more consecutive nucleotides of the sense or antisense sequence of 3′UTR sequence of PKD1 mRNA provided as SEQ ID NOs: 9, 11, or 13. For example, in some aspects the ASOs of the present disclosure hybridize under stringent conditions to at least about 6, 7, 8, 9, 10, 11, 12, 13, 14, 15, 16, or more consecutive nucleotides of the sense or antisense sequence of 3′UTR sequence of PKD1 mRNA provided as SEQ ID NOs: 8, 10, or 12. As a further example, in some aspects the ASOs of the present disclosure hybridize under stringent conditions to the sense or antisense sequence of 3′UTR sequence of PKD1 mRNA provided as SEQ ID NO: 8, 10, or 12.

In certain aspects, the ASO may comprise at least 9, at least 10, at least 11, at least 12, at least 13, at least 14, at least 15, at least 16, at least 17, at least 18, at least 19, or at least 20 nucleotides. In certain aspects, the ASOs may comprise at least 9 nucleotides. In some aspects, the ASOs may comprise 9 to 25 nucleotides, 9 to 24 nucleotides, 9 to 23 nucleotides, 9 to 22 nucleotides, 9 to 21 nucleotides, 9 to 20 nucleotides, 9 to 19 nucleotides, 9 to 18 nucleotides, 9 to 17 nucleotides, 9 to 16 nucleotides 9 to 25 nucleotides, 9 to 24 nucleotides, 9 to 23 nucleotides, 9 to 22 nucleotides, 9 to 21 nucleotides, 10 to 20 nucleotides, 10 to 19 nucleotides, 10 to 18 nucleotides, 10 to 17 nucleotides, 10 to 16 nucleotides, 11 to 25 nucleotides, 11 to 24 nucleotides, 11 to 23 nucleotides, 11 to 22 nucleotides, 11 to 21 nucleotides, 11 to 20 nucleotides, 11 to 19 nucleotides, 11 to 18 nucleotides, 11 to 17 nucleotides, 11 to 17 nucleotides, 12 to 25 nucleotides, 12 to 24 nucleotides, 12 to 23 nucleotides, 12 to 22 nucleotides, 12 to 21 nucleotides, 12 to 20 nucleotides, 12 to 19 nucleotides, 12 to 18 nucleotides, 13 to 25 nucleotides, 13 to 24 nucleotides, 13 to 23 nucleotides, 13 to 22 nucleotides, 13 to 21 nucleotides, 13 to 20 nucleotides, 13 to 19 nucleotides, 13 to 18 nucleotides, or 13 to 17 nucleotides. In some aspects, the ASOs comprise or consist of 9, 10, 11, 12, 13, 14, 15, 16, 17, 18, 19, 20, 21, 22, 23, 24, or 25 nucleotides. In certain aspects, the ASO comprises or consists of 15 or 16 nucleotides.

In certain aspects the ASO may further comprise one or more modifications to a nucleobase, sugar, and/or internucleoside linkage, and as such is a modified oligonucleotide. A modified nucleobase, sugar, or internucleoside linkage may be selected over an unmodified form because of desirable properties such as, for example, enhanced cellular uptake, enhanced affinity for other oligonucleotides or nucleic acid targets, and increased stability in the presence of nucleases. In certain embodiments, a modified nucleoside is a sugar-modified nucleoside. In certain such embodiments, sugar-modified nucleosides may further comprise a natural or modified heterocyclic base moiety or natural or modified internucleoside linkage and may include further modifications independent from the sugar modification. In certain embodiments, a sugar modified nucleoside is a 2′-modified nucleoside, wherein the sugar ring is modified at the 2′ carbon from natural ribose or 2′-deoxy-ribose. In certain embodiments, a 2′-modified nucleoside comprises a 2′-substituent group selected from F, O—CH₃, and OCH₂CH₂OCH₃. In certain embodiments, a 2′-modified nucleoside has a bicyclic sugar moiety. In certain embodiments, a bicyclic sugar moiety comprises a bridge group between the 2′ and the 4′ carbon atoms.

In certain embodiments, a modified oligonucleotide comprises one or more internucleoside modifications. In certain such embodiments, each internucleoside linkage of an oligonucleotide is a modified internucleoside linkage. In certain embodiments, a modified internucleoside linkage comprises a phosphorus atom.

In certain embodiments, a modified oligonucleotide comprises at least one phosphorothioate internucleoside linkage. In preferred embodiments, each internucleoside linkage of a modified oligonucleotide is a phosphorothioate internucleoside linkage.

In certain embodiments, a modified oligonucleotide comprises one or more modified nucleobases. In certain embodiments, a modified oligonucleotide comprises one or more 5-methylcytosines. In certain embodiments, each cytosine of a modified oligo-nucleotide comprises a 5-methylcytosine.

In certain embodiments, a modified nucleobase is selected from 5-hydroxymethyl cytosine, 7-deazaguanine and 7-deazaadenine. In certain embodi-ments, a modified nucleobase is selected from 7-deazaadenine, 7-deazaguanosine, 2-aminopyridine and 2-pyridone.

In some embodiments, the antisense oligonucleotides of the disclosure may be modified at the base moiety, sugar moiety or phosphate backbone to improve, e.g., the stability, hybridization, or solubility of the molecule. By way of another example, the deoxyribose phosphate backbone of the nucleic acids may be modified to generate peptide nucleic acids (see Hyrup et al. (1996) Bioorganic & Medicinal Chemistry 4(I): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of a PNA has been shown to allow for specific hybridization to DNA and RNA under conditions of low ionic strength. The synthesis of PNA oligomers may be performed using standard solid phase peptide synthesis protocols as described in Hyrup et al. (1996) supra; Perry-O'Keefe et al. (1996) Proc. Natl. Acad. Sci. USA 93:14670-675.

In other embodiments, the oligonucleotides of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PKDT Publication No. WO 88/09810) or the blood-brain barrier (see, e.g., PKDT Publication No. WO 89/10134). In addition, oligonucleotides may be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

In certain embodiments, an antisense oligonucleotide of the invention is synthesized with a full phosphorothioate backbone with alternating blocks of 2′-MOE and 2′fluoro sugar-modified nucleosides.

In certain aspects, the ASO may comprise at least one locked nucleic acid (LNA). In some aspects, the ASO may not comprise any locked nucleic acids.

In accord with the foregoing, exemplary ASOs are provided herein. In certain aspects, the ASO of the present disclosure may comprise a nucleic acid sequence having at least 60%, at least 70% or at least 80%, at least 90%, at least 95%, at least 96%, at least 97%, at least 98%, or at least 99% identity to SEQ ID NO: 1, SEQ ID NO: 2, or SEQ ID NO: 3. In some aspects, the ASO of the present disclosure comprises or consists of SEQ ID NO: 1. In some aspects, the ASO of the present disclosure comprises or consists of SEQ ID NO: 2. In some aspects, the ASO of the present disclosure comprises or consists of SEQ ID NO: 3. For ease of reference, exemplary ASOs are provided in the Table 2 below.

TABLE 2
Exemplary anti-sense oligonucleotides

| | | | SEQ |
|---|---|---|---|
| PKD | | | ID |
| Target | Species | Sequence | NO: |
| PKD1 | Mouse | TAAAGTGCTGAAGCC | 1 |
| PKD1 | Human | TTAAAGTGCAGAAGCC | 2 |
| PKD2 | Mouse/ | TAAAGTGCAGAAAATT | 3 |
| | Human | | |

An antisense oligonucleotide of the invention may be synthesized using chemical synthesis and enzymatic ligation reactions using procedures known in the art. For example, an oligonucleotide (e.g., an antisense oligonucleotide) may be chemically synthesized using naturally occurring ribonucleotides, deoxyribonucleotides, variously modified nucleotides designed to increase the biological stability of the molecules or to increase the physical stability of the duplex formed between the antisense and sense nucleic acids, or combinations thereof. For example, phosphorothioate derivatives and acridine substituted nucleotides may be used. Other examples of modified nucleotides which may be used to generate an antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine. Alternatively, the oligonucleotide may be produced biologically using an expression vector into which a nucleic acid has been subcloned in an antisense orientation. Suitable expression vectors are described further below.

### Vectors

In some aspects the current disclosure also encompasses vectors that facilitate transfer of nucleic acids encoding the antisense oligonucleotides into cells, such as, but not limited to, plasmids, transposons, cosmids, chromosomes, artificial chromosomes, viruses, virions, and the like. A vector may also be a chemical vector, such as a lipid complex or naked DNA. In some aspects the vector may be a viral vector. A viral vector may comprise an expression construct as described herein.

A viral vector or a gene therapy vector is a vector that is suitable for gene therapy. Vectors that are suitable for gene therapy are described in Anderson 1998, Nature 392:25-30; Walther and Stein, 2000, Drugs 60:249-71; Kay et al., 2001, Nat. Med. 7:33-40; Russell, 2000, J. Gen. Virol. 81:2573-604; Amado and Chen, 1999, Science 285:674-6; Federico, 1999, Curr. Opin. Biotechnol. 10:448-53; Vigna and Naldini, 2000, J. Gene Med. 2:308-16; Marin et al., 1997, Mol. Med. Today 3:396-403; Peng and Russell, 1999, Curr. Opin. Biotechnol. 10:454-7; Sommerfelt, 1999, J. Gen. Virol. 80:3049-64; Reiser, 2000, Gene Ther. 7:910-3; and references cited therein.

A viral vector and/or a gene therapy vector may be an adenoviral vector, an adeno-associated viral vector or a retroviral vector.

A particularly suitable vector includes an Adenoviral and Adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications. (Russell, 2000, J. Gen. Virol. 81:2573-2604; Goncalves, 2005, Virol J. 2 (1): 43) as indicated above. AAV vectors are even more preferred since they are known to result in very stable long-term expression of transgene expression (up to 9 years in dog (Niemeyer et al, Blood. 2009 Jan. 22; 113 (4): 797-806) and ~2 years in human (Nathwani et al, N Engl J Med. 2011 Dec. 22; 365 (25): 2357-65, Simonelli et al, Mol Ther. 2010 March; 18 (3): 643-50. Epub 2009 Dec. 1.)). Preferred adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6 (5): 482-90, and Martin et al., 2004, Eye 18 (11): 1049-55, Nathwani et al, N Engl J Med. 2011 Dec. 22; 365 (25): 2357-65, Apparailly et al, Hum Gene Ther. 2005 April; 16 (4): 426-34.

Another suitable vector includes a retroviral vector. A preferred retroviral vector for application in the present invention is a lentiviral based viral vector. Lentiviral vectors have the ability to infect and to stably integrate into the genome of dividing and non-dividing cells (Amado and Chen, 1999 Science 285:674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10:448-53) and Vigna et al. (2000, J Gene Med 2000; 2:308-16).

In one aspect, the vector is a lentiviral vector. In another aspect, a single bicistronic viral vector is used. By way of non-limiting example, a single bicistronic lentiviral vector with a 2A self-cleaving peptide sequence, is used as in the experimental section of Xu Y., et al (2019), (Cancer Immunology, Immunotherapy, 68:1979-1993) and Pincha M., et al, (2011), (Gene Therapy, 18:750-764).

Other suitable viral and/or gene therapy vectors include a herpes virus vector, a polyoma virus vector or a vaccinia virus vector.

A viral and/or gene therapy vector comprises a nucleotide encoding an antisense oligonucleotide whereby each of said nucleotide sequence is operably linked to the appropriate regulatory sequences. Such regulatory sequence will at least comprise a promoter sequence. Suitable promoters for expression of such a nucleotide sequence from gene therapy vectors include e.g. cytomegalovirus (CMV) intermediate early promoter, viral long terminal repeat promoters (LTRs), such as those from murine moloney leukemia virus (MMLV) rous sarcoma virus, or HTLV-1, the simian virus 40 (SV 40) early promoter, the MSCV promoter and the herpes simplex virus thymidine kinase promoter. Transposon or other non-viral delivery systems may also be used in this context. All systems can be used in vitro or in vivo.

A viral and/or gene therapy vector may optionally comprise a further nucleotide sequence coding for a further polypeptide. A further polypeptide may be a (selectable) marker polypeptide that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Green (supra).

### Pharmaceutical Compositions

As used herein a “pharmaceutical composition” refers to a preparation of one or more of the active ingredients (including any gene therapy vectors) described herein with other chemical components such as physiologically suitable carriers and excipients. The purpose of a pharmaceutical composition is to facilitate administration of the gene therapy vector to an organism.

Herein the term “active ingredient” refers to the antisense oligonucleotide that is administered to increase PKD1 and/or PKD2 expression in a subject and that is accountable for the biological effect. The term “active ingredient” as used herein can also include a gene therapy vector that encodes the antisense oligonucleotide.

### Pharmaceutically Acceptable Carriers and Excipients

Further aspects of the present disclosure are directed to pharmaceutical compositions comprising at least one antisense oligonucleotide as described above. In various aspects, the pharmaceutical compositions may further comprise a pharmaceutically acceptable carrier or excipient.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

In various embodiments, compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In various embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other embodiments, any of the well-known techniques, carriers, and excipients may be used as suitable and as understood in the art.

In various embodiments, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition.

In various embodiments, pharmaceutical compositions disclosed herein may comprise a viscous formulation. In some aspects, viscosity of the composition may be increased by the addition of one or more gelling or thickening agents. In other aspects, compositions disclosed herein may comprise one or more gelling or thickening agents in an amount to provide a sufficiently viscous formulation to remain on treated tissue. In still other aspects, compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In some embodiments, suitable thickening agent may be carboxymethylcellulose.

In various embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other aspects, one or more of these agents may be added to improve the performance, efficacy, safety, shelf-life and/or other property of the muscarinic antagonist composition of the present disclosure. In s aspects, additives will be biocompatible, and will not be harsh, abrasive, or allergenic

In various embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In various embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In various embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate and sodium metabisulfite and other materials known to one of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more antioxidants by total weight of the composition.

In other embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In another aspect, compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6. In a preferred embodiment, compositions disclosed herein may have a pH greater than about 6.8.

In various embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In other embodiments, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In various embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophanate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In other embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred embodiments, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition.

### Dosage Formulations

In various aspects, the pharmaceutical compositions herein are formulated for systemic administration. Suitable routes of administration may, for example, include parenteral delivery, including intramuscular, subcutaneous and intramedullary injections as well as, intravenous, or intraperitoneal injections. In various aspects, the pharmaceutical compositions are formulated for intravenous, intraperitoneal, or subcutaneous administration.

One may administer the pharmaceutical composition in a local or systemic manner, for example, via local injection of the pharmaceutical composition directly into a tissue region of a patient. In some embodiments, a pharmaceutical composition disclosed herein can be administered parenterally, e.g., by intravenous injection, renal injection, subcutaneous injection, or a combination thereof. In some embodiments, a pharmaceutical composition disclosed herein can be administered orally. In some embodiments, a pharmaceutical composition disclosed herein can administered to the human patient via at least two administration routes. In some examples, the combination of administration routes by be renal injection and intravenous injection; intraperitoneal and intravenous injection; subcutaneous injection and intravenous injection, orally and via renal injection; orally and via intraperitoneal injection; orally and via subcutaneous injection; and orally and via intravenous injection.

Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

Pharmaceutical compositions suitable for use in context of the present disclosure include compositions wherein the active ingredients are contained in an amount effective to achieve the intended purpose. In some embodiments, a therapeutically effective amount means an amount of active ingredients (i.e., those disclosed herein) effective to prevent, slow, alleviate, or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to brain or blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc. Effective doses may be extrapolated from dose-responsive curves derived from in vitro or in vivo test systems

### III. Methods of Use

Further aspects of the present disclosure are directed to method of using the antisense oligonucleotides described above. For example, in some aspects, a method of increasing expression of polycystin 1 (PKD1) in a cell is provided. Other methods include methods of treating autosomal dominant polycystic kidney disease (ADPKD) in a subject in need thereof.

### Methods of Increasing Expression of Polycystin 1 (PKD1) or Polycystin 2 (PKD2)

In various aspects, a method of increasing expression of polycystin 1 and/or polycystin 2 in a cell is provided, the method comprising delivering an ASO as described herein to the cell. In various aspects, the ASO selectively hybridizes to a 3′UTR regulatory region (e.g., cis inhibitory motif) of a PKD1 mRNA and/or PKD2 mRNA, thereby blocking the binding of miR-17 to said region. This stabilizes the mRNA and increases translation and ultimate expression of the encoded protein (polycystin 1 or polycystin 2).

In various aspects, the cell may carry a mutation in at least one allele of the PKD1 and/or PKD2 gene. In various aspects, the cell may be heterozygous for a mutant PKD1 and/or PKD2 allele. In various aspects, the mutated PKD1 or PKD2 allele may be a loss of function allele that does not express a functional protein. In some aspects, the cell may have the mutant (LOF) allele and a second allele that is WT. Alternatively, the cell may have a mutant (LOF) allele and a second allele comprising a different mutation that further reduces polycystin 1 and/or polycystin 2 expression (but does not eliminate it). For example, the second allele may comprise a missense mutation that destabilizes mRNA transcribed from it, resulting in reduced protein expression. In general, the cell has or is genetically engineered to have reduced expression of polycystin 1 (PKD1) and/or polycystin 2 (PKD2) as compared to a normal WT cell (i.e, a cell comprising two normal alleles of PKD1 or PKD2). In various aspects, the cell expresses polycystin 1 to a level that is less than 80%, less than 75%, less than 70%, less than 65%, less than 60%, less than 55%, less than 50%, less than 45%, less than 40%, less than 35%, less than 30%, less than 25%, or less than 20% the level expressed by a WT cell. In various aspects, the cell expresses polycystin 2 to a level that is less than 80%, less than 75%, less than 70%, less than 65%, less than 60%, less than 55%, less than 50%, less than 45%, less than 40%, less than 35%, less than 30%, less than 25%, or less than 20% the level expressed by a WT cell.

In various aspects, the method comprises increasing expression of PKD1 in the cell by at least 20%, at least 25% at least 30%, at least 40%, at least 50%, at least 60%, at least 70% or at least 80% over baseline, where baseline is defined as the level of expression in the cell under normal conditions. For example, in certain aspects the method comprises increasing expression of PKD1 by at least 25% over baseline.

In various aspects, the cell may be an epithelial, endothelial (e.g., vascular endothelial), immune, or stromal cell. In some aspects, the cell may be a kidney cell or a kidney cell line (e.g., kidney epithelial cell, an inner medullary collecting duct (IMCD) cell, HK-2 cells, renal cortical epithelial cells, renal medullary epithelial cells, renal mixed epithelial cells, or renal proximal tubule epithelial cell). For example, the cell can be a kidney epithelial cell or a mIMCD3 cell. In certain aspects, the cell may be obtained from a patient with autosomal dominant polycystic kidney disease. The cell may be human or murine.

In various aspects, the method of increasing PKD1 expression is performed in vitro. For example, certain aspects of the present disclosure are directed to increasing PKD1 and/or PKD2 expression in a cell line in vitro. The cell line may be an immortalized cell line generated from a human tissue sample (e.g., from a patient with ADPKD). Alternatively, the cell line may be generated from a genetically engineered mouse model (i.e., a Pkd1^{RC/-} cell line). Pkd1^{RC/-} cell lines are described in more details in the Examples herein below.

In various aspects, the method of increasing PKD1 and/or PKD2 expression is performed in vivo. For example, the cell may be in vivo (i.e., in a patient having or autosomal dominant polycystic kidney disease).

In various aspects, delivering the antisense oligonucleotide (ASO) may comprise delivering an expression vector encoding the ASO to the cell. Suitable viral vectors that may be used to this end are described above.

### Methods of Treating Autosomal Dominant Polycystic Kidney Disease

In still further aspects, a method of treating autosomal dominant polycystic kidney disease is provided, the method comprising administering an antisense oligonucleotide (ASO) to the subject in need thereof. The method may further comprise increasing endogenous expression of polycystin 1 and/or polycystin 2 in at least one cell in the subject.

In various aspects, the ASO is administered as part of a pharmaceutical composition, such as any described herein. In some aspects, the ASO is administered systemically (i.e., intravenously, subcutaneously, or intraperitoneally). In each of these embodiments, the ASO may be prepared as a pharmaceutical formulation tailored to each administration route.

In various aspects, administering the ASO can comprise administering an expression construct or vector (e.g., a viral vector) that encodes the ASO and allows for expression of the ASO in a cell of the subject.

In various aspects, treating a subject with ADPKD results in at least 25%, at least 30%, at least 40%, 50%, at least 60%, at least 70%, at least 80%, at least 90%, or at least 100% polycystin 1 (PKD1) and/or polycystin 2 (PKD2) expression in at least one cell of the subject as compared to baseline PKD1 and/or PKD2 expression in a patient with ADPKD.

A suitable subject includes a human, a livestock animal, a companion animal, a lab animal, or a zoological animal. In one embodiment, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoologicalanimal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

### EXAMPLES

### Example 1—Introduction to Examples

An estimated 12.5 million people worldwide suffer from autosomal dominant polycystic kidney disease (ADPKD), making it among the most common monogenetic conditions known to humankind. A clinical hallmark of ADPKD is the relentless growth of innumerable fluid-filled cysts in the kidneys, which replace the normal parenchyma and, over decades, cause massive bilateral kidney enlargement and renal failure. ADPKD occurs because of heterozygous, loss-of-function mutations in PKD1 (~78% of cases) or PKD2 (~15% of cases). The classical hypothesis for cyst initiation is that in addition to a germline inactivating mutation in one allele of the PKD gene, there is somatic inactivation (referred to as the second hit) in the other allele, causing a complete loss of polycystin expression in the cell. However, in recent years, several lines of evidence support the gene dose threshold as a mechanism involved in cystogenesis. This hypothesis posits that complete PKD1 loss is not necessary, but rather cystogenesis ensues if the functional PKD1 dosage falls below a critical threshold. Supporting the gene dosage model, inactivating second hit mutation is not a universal feature, especially in smaller ADPKD cysts. Importantly, many individuals with ADPKD continue to have residual PKD1 expression because they carry missense (rather than inactivating) germline PKD1 mutations. As proof of principle, lowering the Pkd1 dose is sufficient to produce PKD in mice, pigs, and monkeys. Thus, if reduced dosage causes ADPKD, increasing the expression of the normal PKD1 allele could arrest the disorder. However, despite this transformative potential, the factors governing PKD1 dosage in ADPKD are mostly unknown, and currently, there are no mechanisms to activate the normal PKD1 allele.

The 3′-untranslated region (3′-UTR), the mRNA portion that lies immediately downstream of the translation termination codon, protects the mRNA from degradation and facilitates translation through its poly(A) tail. Paradoxically, the 3′-UTR, via interaction with microRNAs (miRNAs), can also mediate mRNA translation repression or deadenylation. Most mRNA 3′-UTRs harbor evolutionarily conserved miRNA-binding elements (MBEs), implying that cis-inhibition of translation is a pervasive mode of gene output regulation. However, this intriguing aspect of the 3′-UTR function is poorly delineated. The prediction is that individual MBEs have a minor impact on host mRNA function, considering that miRNAs mostly act as rheostats and modestly repress mRNA targets. Counter to this prevailing logic, we reasoned that under certain circumstances, such as when gene dosage is already reduced due to haploinsufficiency, MBE-mediated cis-inhibition of the remaining allele could have a disease-modifying effect by governing the final protein output.

The goal in the following examples was to determine whether monoallelic PKD1 derepression is possible and how it influences preclinical ADPKD progression. PKD1 contains a miR-17 binding motif in its 3′-UTR, and miR-17 expression and activity are higher in ADPKD models. Therefore, whether PKD1 mRNA is cis-inhibited by its 3′-UTR miR-17 motif was tested along with the idea that blocking this inhibition reverses PKD1 decline. CRISPR/Cas9 editing was used to delete the miR-17 motif from the PKD1 gene in monoallelic ADPKD models. It was found that eliminating the miR-17 motif is sufficient to improve Pkd1 mRNA stability, raise Polycystin-1 (PKD1) expression, and ameliorate cyst growth in cellular, ex vivo, and mouse PKD models. The other ADPKD gene, PKD2, also contains a 3′-UTR miR-17 binding motif; remarkably, deleting this miR-17 motif increases Polycystin-2 (PKD2) levels and attenuates cyst growth in Pkd1-mutant models. Furthermore, acute pharmaceutical blockade of Pkd1/2 cis-inhibition prevents cyst onset and stabilizes established PKD in mice. Finally, it was demonstrated that PKD1 or PKD2 derepression reverses cyst-pathogenic events in primary kidney cyst epithelia derived from individuals with ADPKD.

### Example 2—Pkd1 is Cis-Repressed Via its 3′-UTR miR-17 Binding Motif

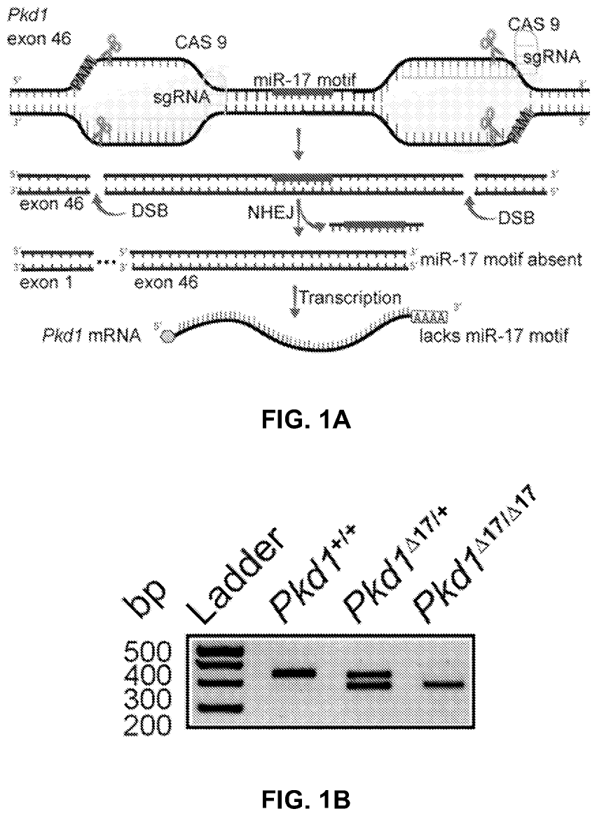

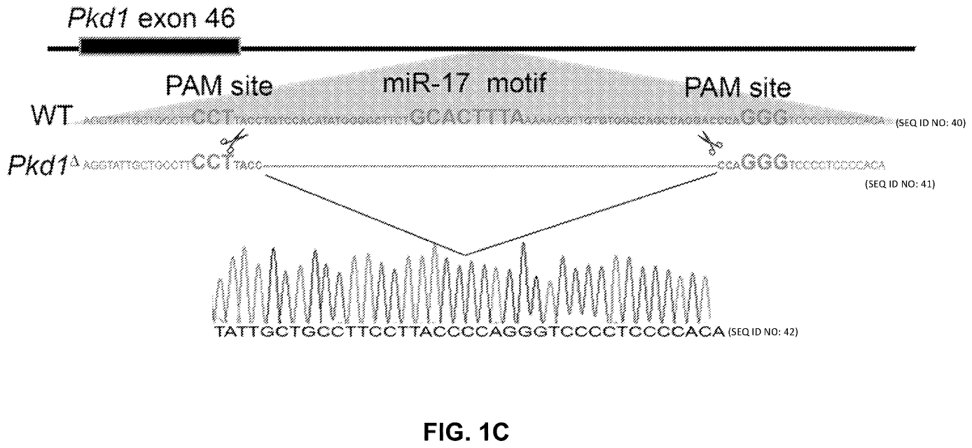

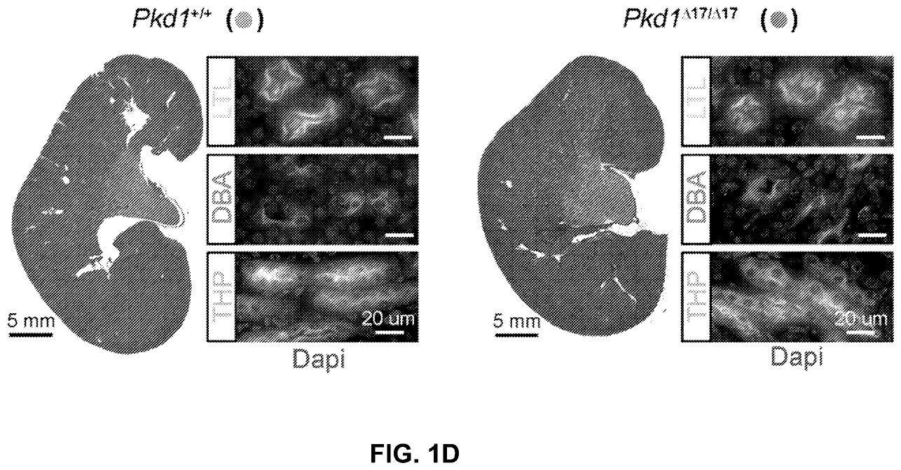

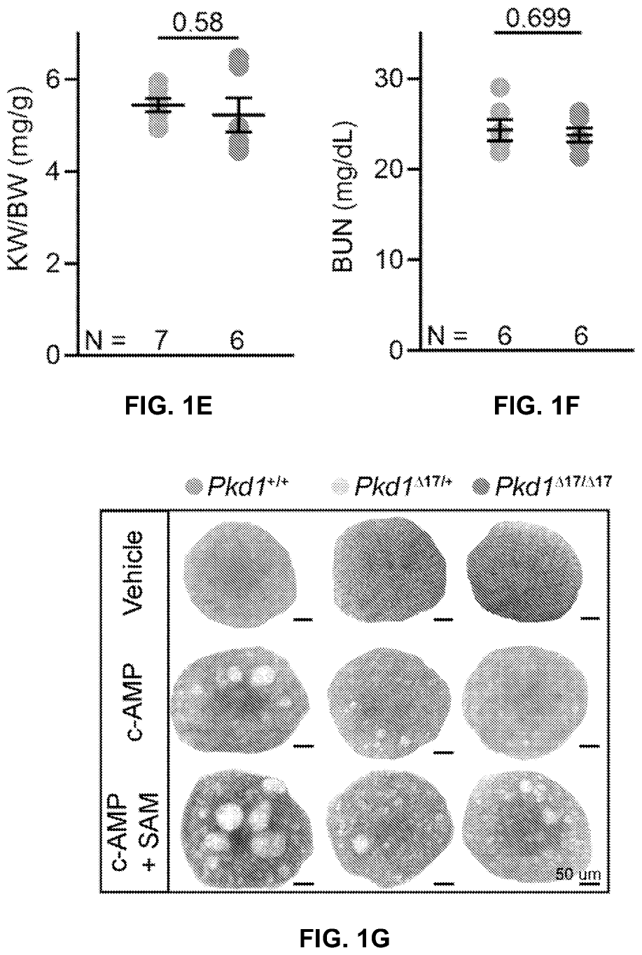

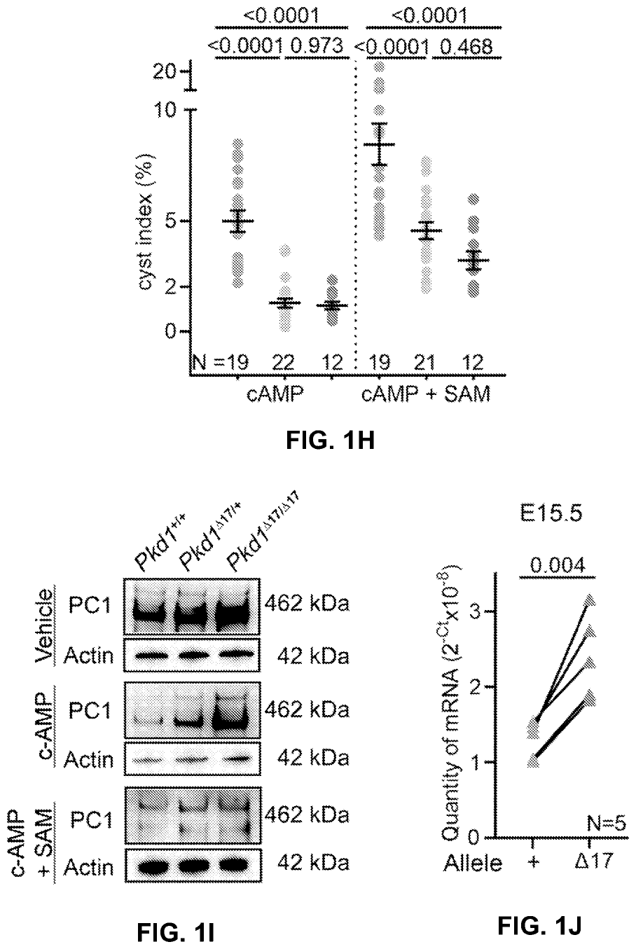

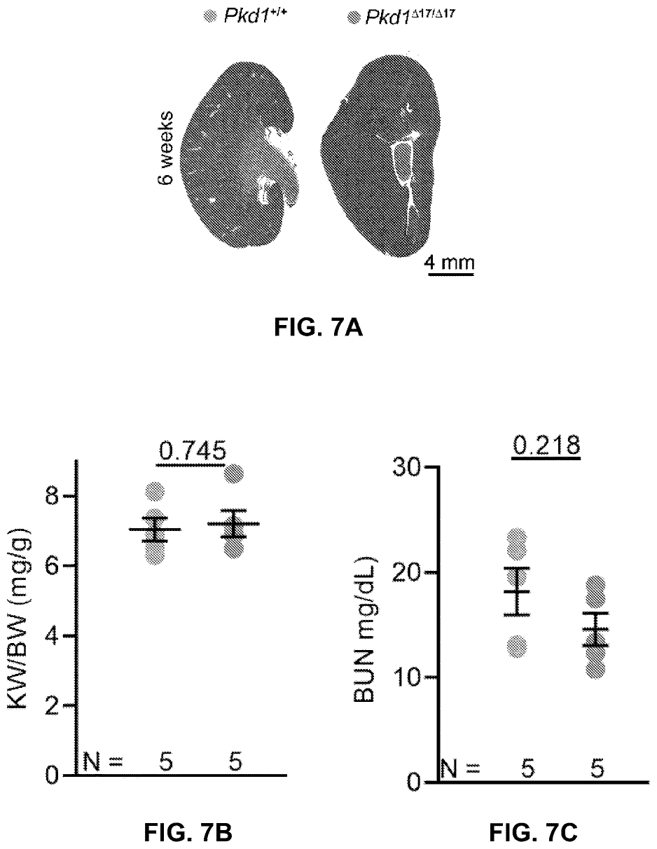

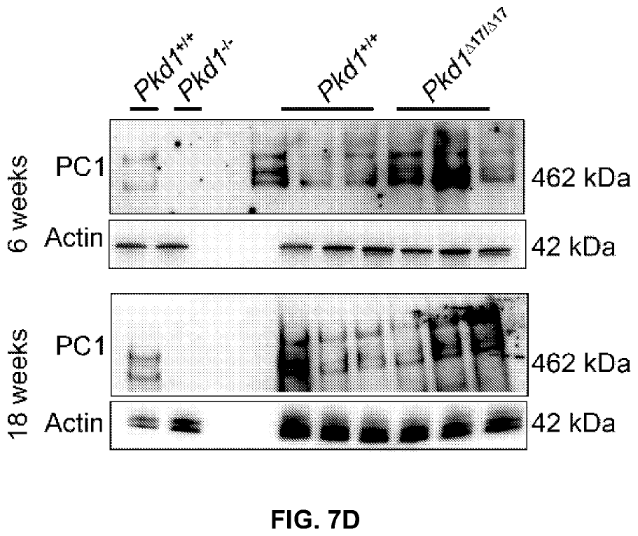

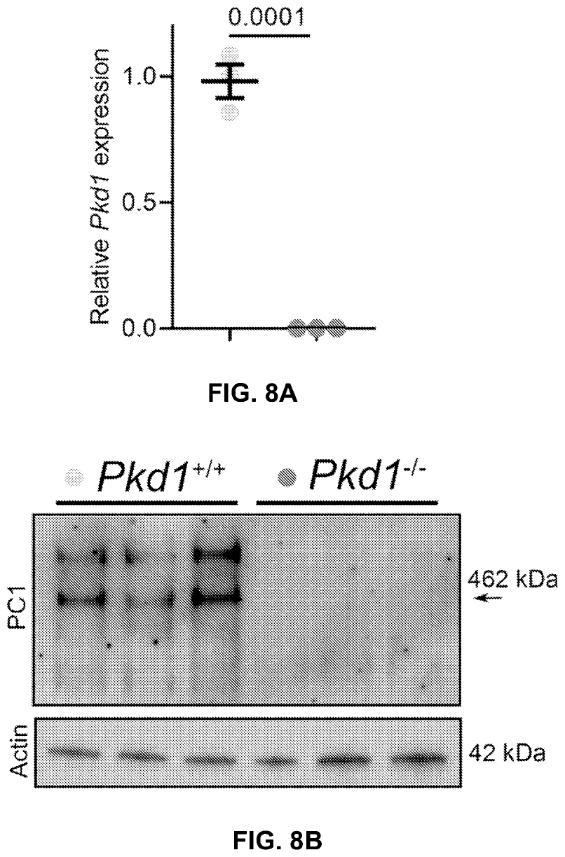

In this example, data is presented showing that cis-repression via a 3′-UTR miR-17 binding motif on the Pkd1 mRNA can govern Pkd1 dosage. Initial experiments tested the impact of deleting this MBE in normal mouse kidneys. sgRNAs were designed that bind to Pkd1 exon-46, flanking the DNA segment that encodes the miR-17 motif, and CRISPR/Cas9 editing was used to generate Pkd1 alleles (Pkd1^{Δ17}) lacking the miR-17 binding site (FIG. 1A). The motif deletion was validated by using DNA PKDR followed by direct Sanger sequencing (FIG. 1B-1C). This editing approach did not inadvertently inactivate Pkd1 since normal kidney histology and renal function was observed in 6-week-old and 18-week-old Pkd1^{Δ17/Δ17} mice (FIG. 1D-1F and FIG. 7A-7D. Western blot analysis using the 7E12 PKD1 antibody, which detects the full-length protein and the n-terminal fragment (see methods for details) was used to assess PKD1 levels. The 7E12 antibody was validated in wildtype and Pkd1″-cells. As expected, no PKD1 signal was observed in the Pkd1-null cells (FIG. 8A-8B). Despite the loss of the miR-17 binding site, PKD1 expression was the same between the kidneys of 6-week-old or 18-week-old Pkd1^{Δ17/Δ17} mice and their respective age-matched control Pkd1^{+/+} mice (FIG. 7D), implying that there was no Pkd1 cis-inhibition in the kidneys of normal adult mice.

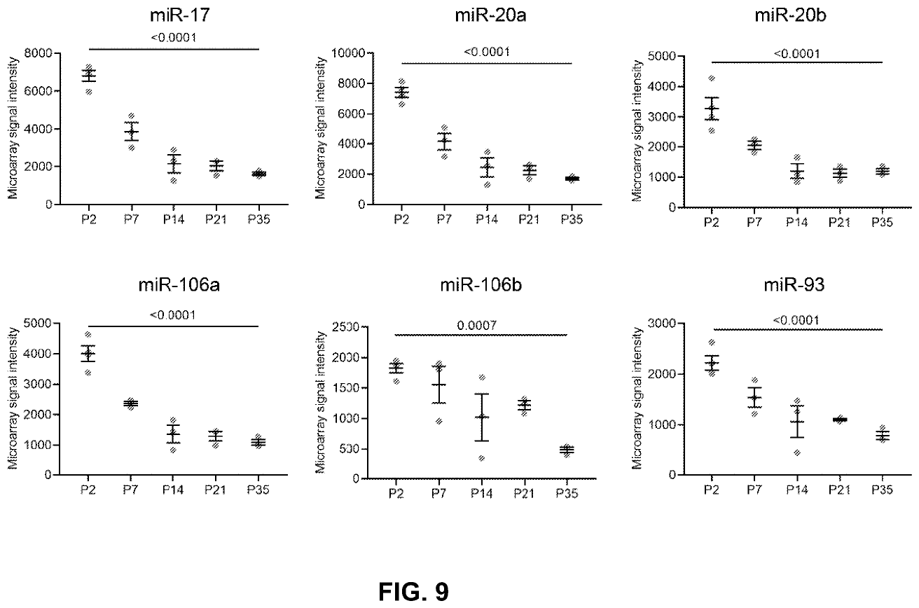

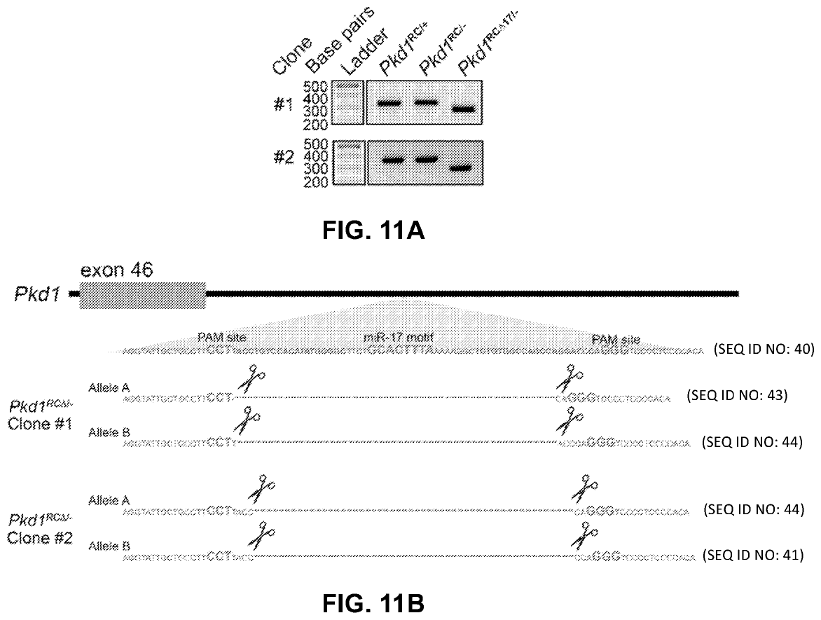

Analysis of the miRNA microarray dataset revealed that miR-17 levels decline with postnatal maturation (FIG. 9). Thus, the lack of Pkd1 cis-inhibition in mature kidneys is likely due to the low basal miR-17 activity. Therefore, embryonic (E) Pkd1^{Δ17/+} and Pkd1^{Δ17}/Δ17 kidneys were analyzed. Specifically, ex vivo kidney organ culture was analyzed to simultaneously assess the impact on Pkd1 expression and cystogenesis. E13.5 littermate Pkd1^{+/+}, Pkd1^{Δ17/+}, and Pkd1^{Δ17/Δ17} kidneys were cultured for four days in media containing 100 UM 8-bromo-cAMP or 100 μM 8-bromo-cAMP plus S-adenosylmethionine (SAM), or vehicle control (FIG. 1G-1H). CAMP increased cyst formation in Pkd1^{+/+} kidneys compared to vehicle treatment, and this effect was further enhanced with the addition of SAM. Interestingly, the pro-cytogenic effect of CAMP and SAM was attenuated in Pkd1^{Δ17/+} and Pkd1^{Δ17/Δ17} kidneys (FIG. 1G-1H). Moreover, higher PKD1 expression in Pkd1^{Δ17/+} and Pkd1^{Δ17/Δ17} was observed compared to Pkd1^{+/+} ex vivo cultured kidneys by immunoblot analysis (FIG. 11). These data indicate that miR-17 motif deletion derepresses PKD1 and blocks the pro-cytogenic effect of CAMP in embryonic cultured kidneys.

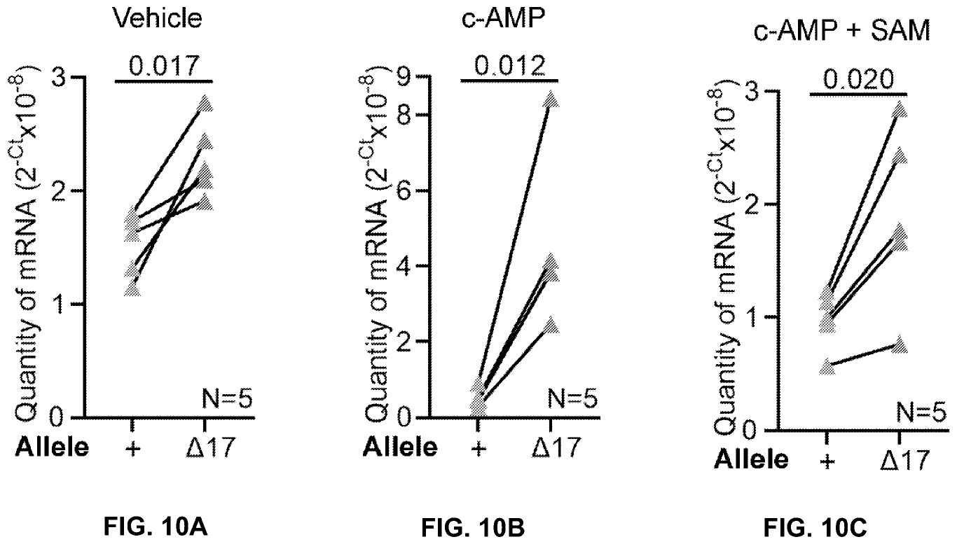

Next, the relative abundance of wildtype and Δ17 transcripts within the total Pkd1 mRNA pool was measured. Allele-specific primers were designed to take advantage of the unique mRNA sequence created by CRISPR/Cas9 editing of the Δ17 allele. qRT-PKDR revealed that in E15.5 heterozygous in vivo Pkd1^{Δ17/+} kidneys, the Δ17 allele contributed nearly 50% more transcripts than its wildtype counterpart (FIG. 1J). Similarly, it was noted that the Δ17 allele produced more Pkd1 mRNAs than the wildtype allele in ex vivo Pkd1^{Δ17/+} kidney cultures. This difference became even more pronounced in the presence of CAMP (FIG. 10A-10C). These observations further imply inhibition of wildtype Pkd1 mRNAs by miR-17 but an evasion of repression and improved stability of Pkd1^{Δ17} mRNAs in embryonic kidneys.

### Example 3—Endogenous Monoallelic Pkd1 Derepression Alleviates Polycystic Kidney Disease

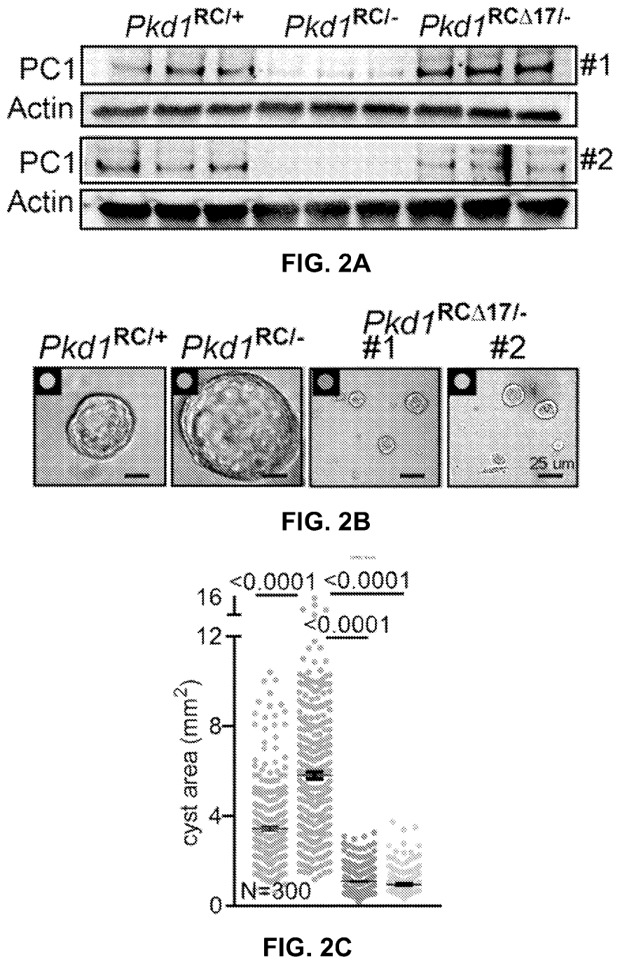

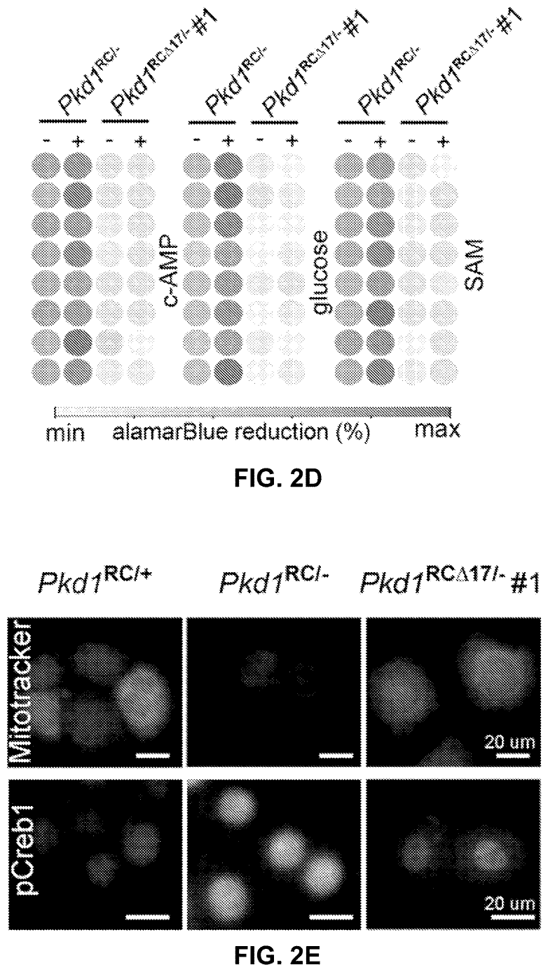

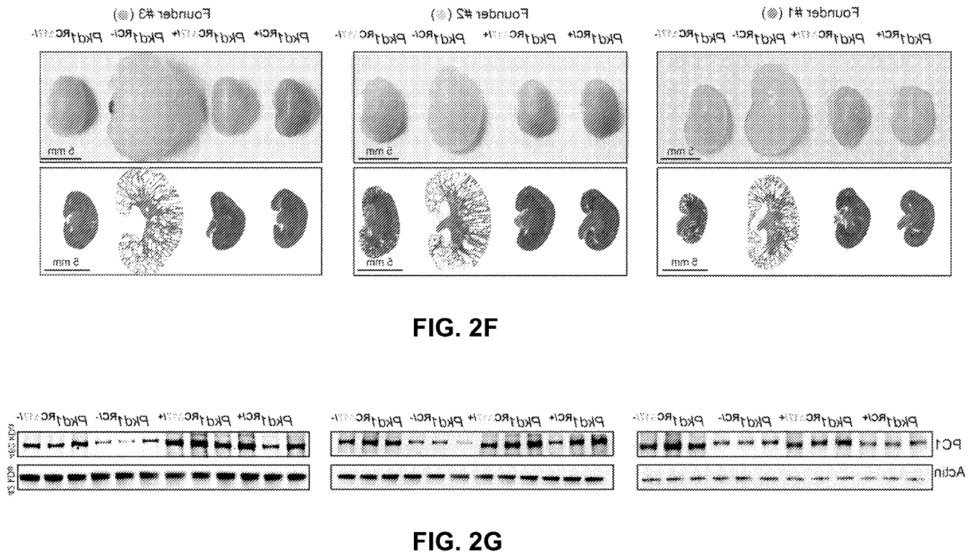

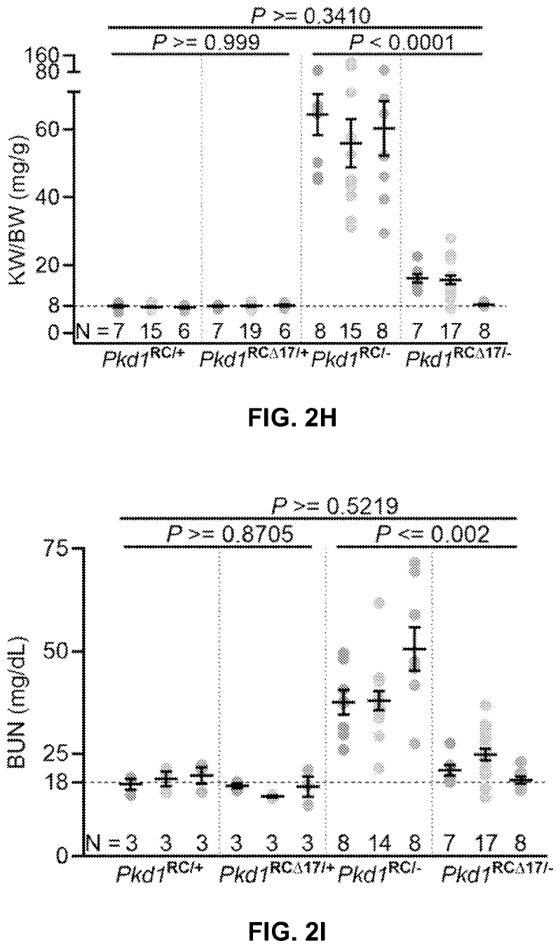

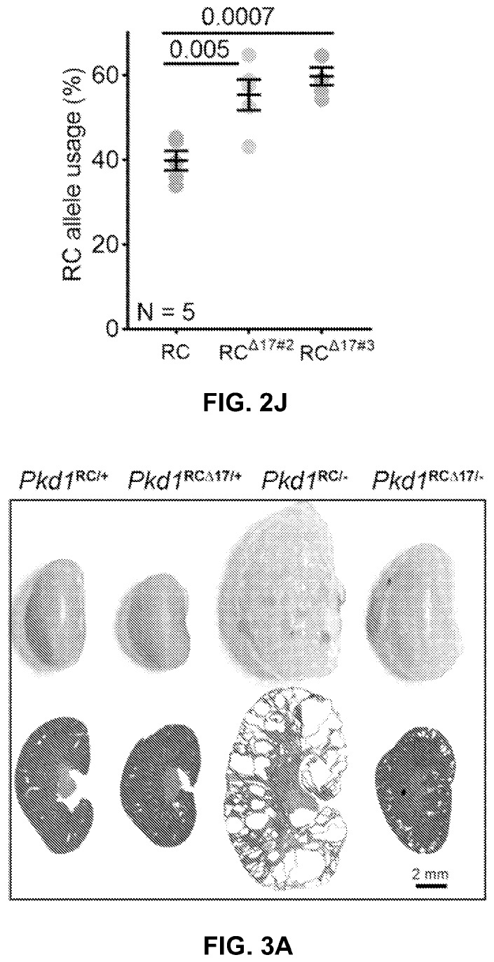

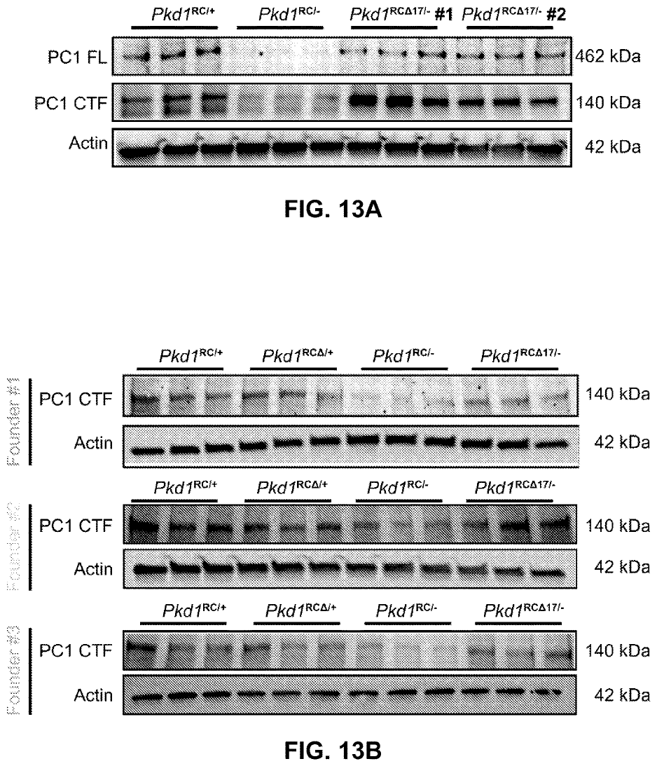

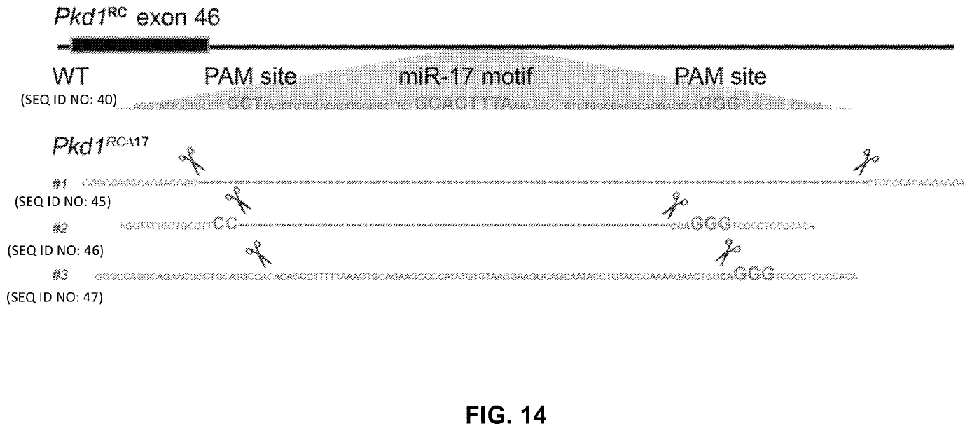

In this example, data is presented showing that Pkd1 is cis-repressed in ADPKD and that preventing this inhibition has a disease-modifying impact. This is significant because kidney cyst formation ensues when PKD1 dosage falls below a critical threshold and no approach exists to reverse the PKD1 decline. Initially, the Pkd1^{RC/-} cellular ADPKD model was examined. This is a collecting duct-derived mouse cell line that harbors the missense RC mutation on one Pkd1 allele, whereas the other allele is inactivated (Lakhia, R. et al., “Enhancer and super-enhancer landscape in polycystic kidney disease” BioRxv, 2021 biorxiv.org/content/10.1101/2021.11.19.469306v1.full.pdf, which is incorporated herein by reference in its entirety). The mutation results in arginine to cystine substitution two amino acids before the second transmembrane domain and reduces mature (functional) PKD1 protein levels. The RC mutation maps to Pkd1 exon-30 and is significantly upstream of the miR-17 motif, which is encoded by Pkd1 exon-46. This allows CRISPR/Cas9 editing to remove the 3′-UTR miR-17 motif from the RC allele (Pkd1^{RCΔ17/-}) (FIG. 11A-11B). Next, two independent Pkd1^{RCΔ17/-} clonal cell lines were generated and characterized in relation to the unedited parental Pkd1^{RC/-} and Pkd1^{RC/+} cells. It was previously reported that PKD1 expression was reduced in Pkd1^{RC/-} cells compared to Pkd1^{RC/+} cells. Remarkably, western blot analysis using the 7E12 antibody revealed that eliminating the miR-17 motif restored PKD1 expression in Pkd1^{RCΔ17/-} cell lines (FIG. 2A and FIG. 13A). Next, employing several independent assays, it was demonstrated that this degree of PKD1 derepression was sufficient to reverse several well-known pathogenic events linked to cyst growth. First, Pkd1^{RC/-} cells had a higher proliferation rate and 3D cyst size than Pkd1^{RC/+} cells, and this discrepancy was normalized after PKD1 derepression in Pkd1^{RCΔ17/-} cells (FIG. 2B-2C). Second, it was observed that while CAMP, glucose, and SAM increased the already elevated proliferation of Pkd1^{RC/-} cells, Pkd1^{RCΔ17/-} cells were resistant to these pro-proliferative stimuli (FIG. 2D and Table 3, below).

TABLE 3
alamarBlue assay on Pkd1^{RC/-} and Pkd1^{RCD17/-} #1 cells.

| | Cell line | | | | | | | | | | | |
|---|---|---|---|---|---|---|---|---|---|---|---|---|
| | Pkd1^{RC/-} | | Pkd1^{RCD17/-} #1 | | Pkd1^{RC/-} | | Pkd1^{RCD17/-} #1 | | Pkd1^{RC/-} | | Pkd1^{RCD17/-} #1 | |
| | Stressor | | | | | | | | | | | |
| | Ctl | cAMP | Ctl | cAMP | Ctl | Gluc | Ctl | Gluc | Ctl | SAM | Ctl | SAM |
| Replicate | | | | | | | | | | | | |
| 1 | 40.19 | 46.67 | 30.04 | 28.88 | 38.07 | 49.99 | 27.39 | 27.35 | 38.58 | 47.47 | 26.87 | 29.91 |
| 2 | 39.75 | 45.56 | 25.8 | 25.63 | 37.34 | 49.78 | 31.51 | 27.74 | 39.09 | 46.37 | 25.20 | 26.70 |
| 3 | 40.32 | 41.16 | 26.19 | 26.97 | 38.19 | 47.97 | 28.27 | 27.03 | 39.29 | 42.01 | 27.25 | 28.02 |
| 4 | 37.42 | 42.82 | 27.03 | 27.97 | 37.78 | 44.98 | 27.62 | 26.04 | 37.77 | 43.66 | 28.09 | 29.01 |
| 5 | 38.77 | 40.43 | 27.26 | 28.99 | 34.85 | 43.70 | 27.25 | 28.45 | 37.33 | 41.29 | 28.32 | 30.03 |
| 6 | 36.75 | 42.37 | 27.79 | 28.19 | 38.74 | 43.92 | 25.34 | 28.74 | 40.58 | 43.21 | 28.85 | 29.24 |
| 7 | 37.98 | 44.11 | 25.84 | 26.91 | 40.26 | 46.17 | 25.54 | 29.29 | 39.85 | 44.93 | 26.91 | 27.97 |
| 8 | 39.15 | 41.98 | 27.92 | 22.64 | 38.77 | 48.13 | 28.19 | 29.96 | 39.51 | 42.83 | 28.98 | 23.72 |
| P value | | | | | | | | | | | | |

| | | | |
|---|---|---|---|
| Ctl vs | 0.0002 | <0.0001 | <0.0001 |
| Stressor | | | |

| | | | |
|---|---|---|---|
| Ctl vs | .9952 | .7041 | .9296 |
| Stressor | | | |

| | | | |
|---|---|---|---|
| Ctl vs Ctl | <0.0001 | <0.0001 | <0.0001 |

Raw values of percentage reduction are shown for each biological replicate. The results of one-way ANOVA followed by post-hoc Tukey's multiple comparisons test are listed.

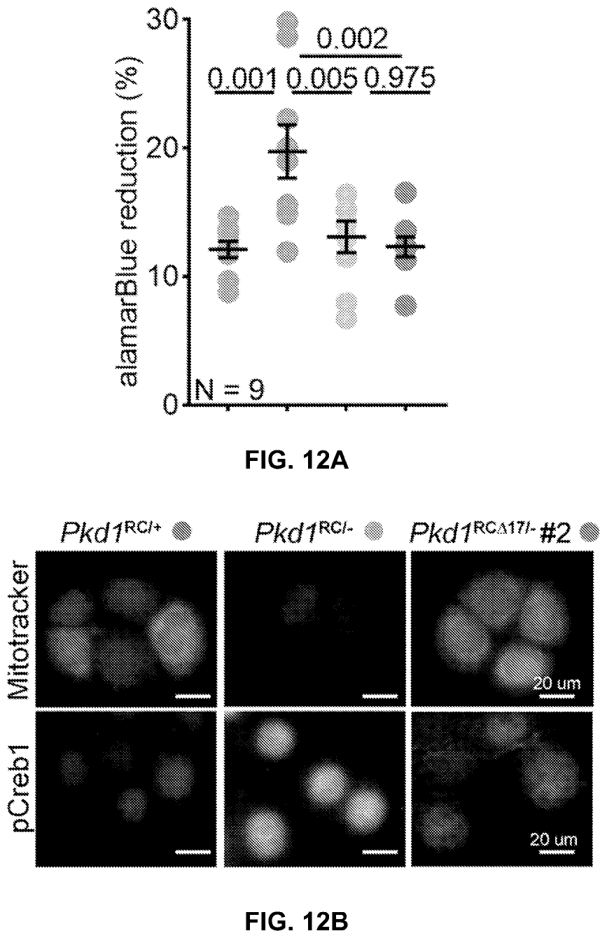

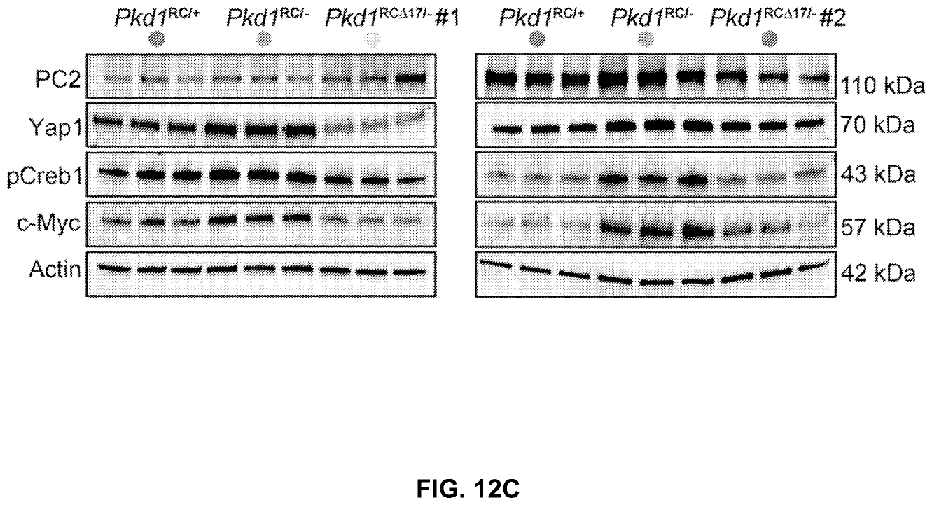

Third, MitoTracker was used to assess mitochondrial membrane potential as a proxy of oxidative phosphorylation and anti-PKDreb1 antibody immunofluorescence as a readout of c-AMP signaling. Compared to Pkd1^{RC/+} cells, we observed a reduced MitoTracker signal and higher PKDreb1 expression in Pkd1^{RC/-} cells. The opposite was true for Pkd1^{RCΔ17/-} cells, which exhibited restored MitoTracker signal and lowered PKDreb1 expression (FIG. 2E and FIG. 12B). Finally, immunoblot analysis revealed elevated Yap1, PKDreb1, and c-Myc expression in Pkd1^{RC/-} cells compared to Pkd1^{RC/+} cells, which returned to baseline in Pkd1^{RCΔ17/-} cells (FIG. 12C).

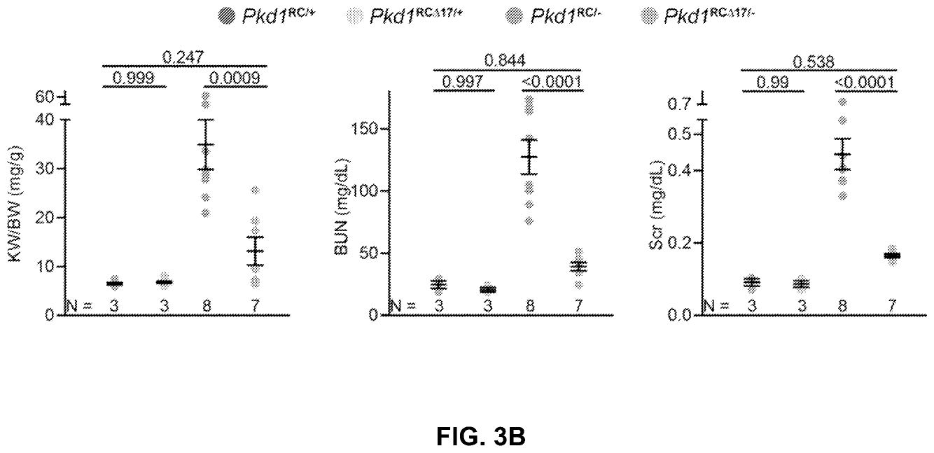

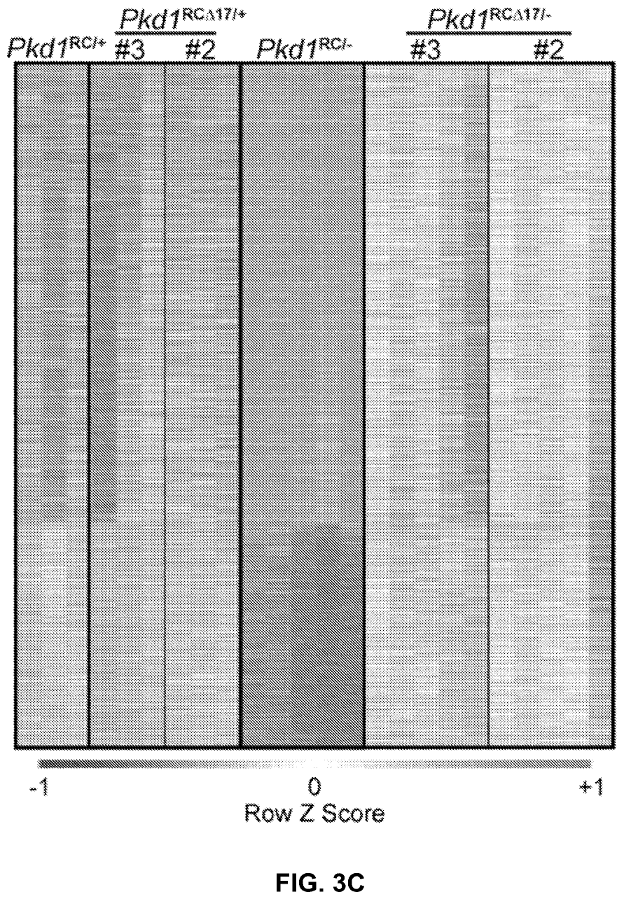

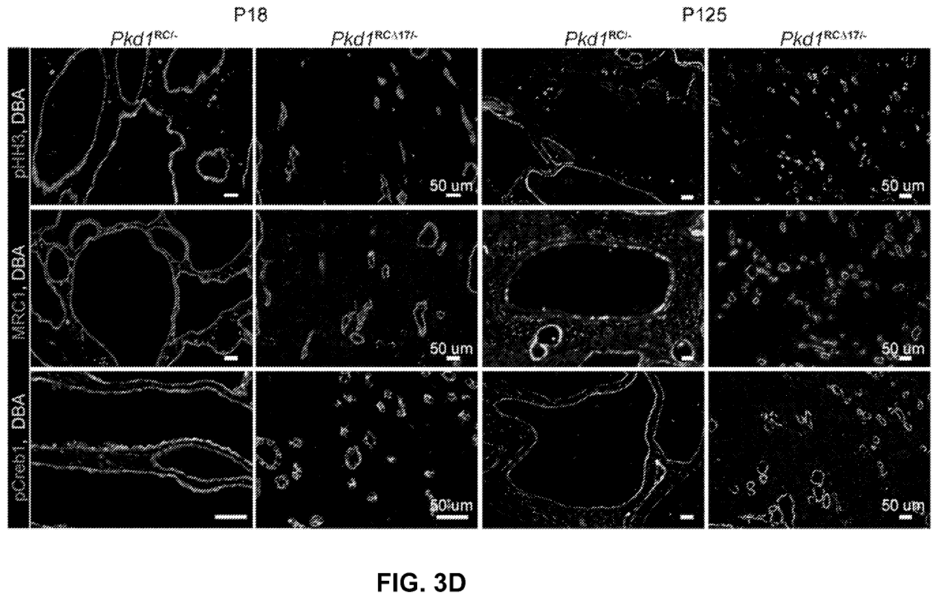

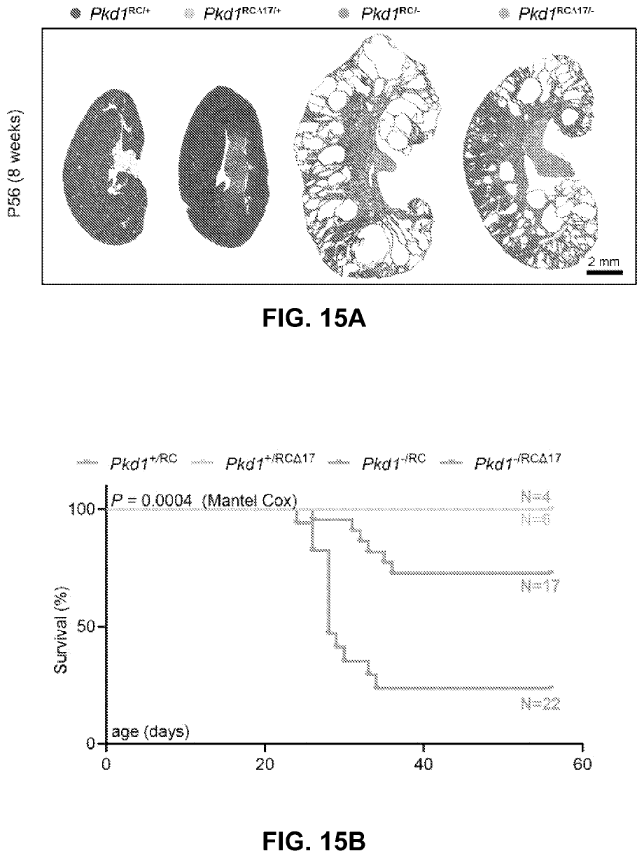

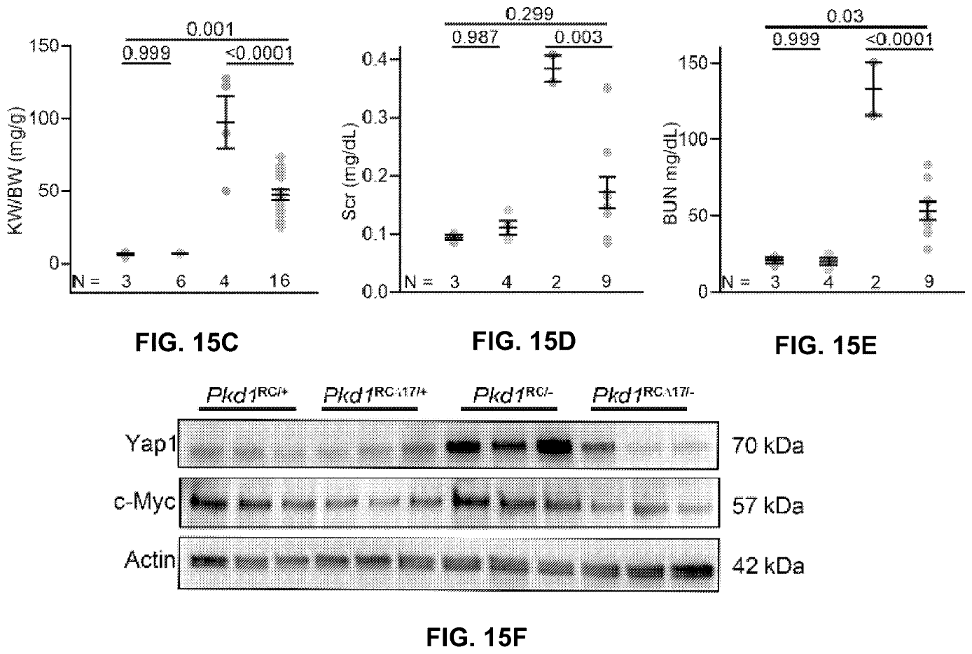

Based on these encouraging results, the 3′-UTR Δ17 deletions were then modeled in vivo. CRISPR-edited KsPKDret; Pkd1^{RC/RC} fertilized eggs were prepared to eliminate the miR-17 motif from the Pkd1 RC allele. These eggs were then implanted into pseudopregnant surrogate female mice and eventually generated three germline-transmitting heterozygous KsPKDret; Pkd1^{RCΔ17}/RC founder mice. Using DNA, PKDR and Sanger sequencing, it was determined that the miR-17 motif was indeed deleted in all three founder mice from one RC allele, whereas the other RC allele still contained the wildtype 3′-UTR (FIG. 14). Mice with heterozygous Δ17 deletions were selected because breeding them with Pkd1^{F/F} mice allowed the generation of the following four relevant genotypes from the same breeding pair: Pkd1^{RC/F} (Pkd1^{RC/+}), Pkd1^{RCΔ17/F} (Pkd1^{RCΔ17/+}), KsPKD^{re+}; Pkd1^{RC/F} (Pkd1^{RC/-}), and KsPKD^{re+}; Pkd1^{RCΔ17/F} (Pkd1^{RCΔ17/-}). Data from the 18-day-old progeny of all three founders are shown in FIG. 2F-2H. First, it was noted that Pkd1^{RCΔ17}/+ mice maintained normal kidney histology and function, again indicating that miR-17 motif deletion does not disrupt Pkd1 or produce PKD (FIG. 2F). For each founder progeny, reduced PKD1 expression, severe cystic kidney disease, an increased kidney-weight-to-body-weight (KW/BW) ratio, and higher serum BUN levels was observed in Pkd1^{RC/-} mice than in Pkd1^{RC/+} mice (FIG. 2F-2J). As with the cell lines, miR-17 motif deletion caused PKD1 derepression, as assessed using the 7E12 antibody, in Pkd1^{RCΔ17/-} kidneys compared to Pkd1^{RC/-} kidneys (FIG. 2G). PKD1 derepression in cell lines and mice was verified using a second independent antibody generated by the U Maryland PKD center (see Example 8 for details) that detects the PKD1 c-terminus (FIG. 13A-13B). Moreover, using paired-end RNA-seq, higher RC allele usage was observed in Pkd1^{RCΔ17/-} kidneys than in Pkd1^{RC/-} kidneys, further indicating Pkd1 derepression (FIG. 2J). Strikingly, the cystic disease was almost completely alleviated, and KW/BW and serum BUN were nearly normalized in Pkd1^{RCΔ17/-} mice compared to Pkd1^{RC/-} mice (FIG. 2F-I). To examine the long-term effects, founder #2 and #3 progeny were prospectively followed for 8 and 18 weeks, respectively. Founder #2 progeny exhibited an aggressive cystic disease phenotype, with 76.4% (13/17) of Pkd1^{RC/-} mice succumbing to kidney failure before eight weeks of age (FIG. 15A-15B). Moreover, the four surviving Pkd1^{RC/-} mice exhibited severe PKD and near-fatal kidney failure. In contrast, only 27.2% (6/22) of Pkd1^{RCΔ17/-} mice died by eight weeks, and the surviving mice had fewer cysts and relatively preserved kidney function (FIG. 15C-15E). All founder #3 Pkd1^{RC/-} progeny survived until 18 weeks of age. However, they developed progressive kidney failure, as evidenced by an average blood urea nitrogen (BUN) of >100 mg/dl and serum creatinine of >0.4 mg/dl (FIG. 3B). Founder #3 Pkd1^{RCΔ17/-} mice exhibited minimal disease progression with average BUN<30 mg/dl and serum creatinine<0.2 mg/dl (FIG. 3A-3B).

Large-scale transcriptomic dysregulation, activation of tubular proliferation and oncogenic signaling, and interstitial inflammation are some of the key pathological hallmarks of ADPKD. Therefore, it was next addressed whether these changes were blunted by PKD1 derepression. RNA-seq analysis was performed using kidney samples from 18-day-old Pkd1^{RC/+}, Pkd1^{RCΔ17/+}, Pkd1^{RC/-}, and Pkd1^{RCΔ17/-} mice. Dysregulation of an extensive network of gene transcripts was observed with upregulation of 4157 and downregulation of 2067 mRNAs in cystic Pkd1^{RC/-} compared to noncystic Pkd1^{RC/+} control kidneys (FIG. 3C). Mirroring kidney histology, Pkd1^{RCΔ17/+} exhibited a nearly identical gene expression pattern as Pkd1^{RC/+} kidney. Impressively, >95% of dysregulated mRNAs in Pkd1^{RC/-} kidneys showed improved (or normalized) expression in Pkd1^{RCΔ17/-} kidneys (FIG. 3C). Consistent with the RNA-seq data, immunoblot analysis revealed reduced c-Myc and Yap1 in the kidneys of 18-day-old Pkd1^{RCΔ17/-} mice compared to Pkd1^{RC/-} mice (FIG. 15F). Finally, immunofluorescence analysis demonstrated fewer anti-phospho-Histone-H3-positive cells, indicating lower proliferation, and reduced anti-PKDreb1 and anti-MRC1 signals, implying attenuated c-AMP signaling and cyst-associated inflammation, respectively, in kidneys of 18-day-old and 18-week-old Pkd1^{RCΔ17/-} mice compared to Pkd1^{RC/-} mice (FIG. 3D).

### Example 4—Preventing Pkd2 Cis-Inhibition Attenuates Cyst Growth in Pkd1-Mutant Models

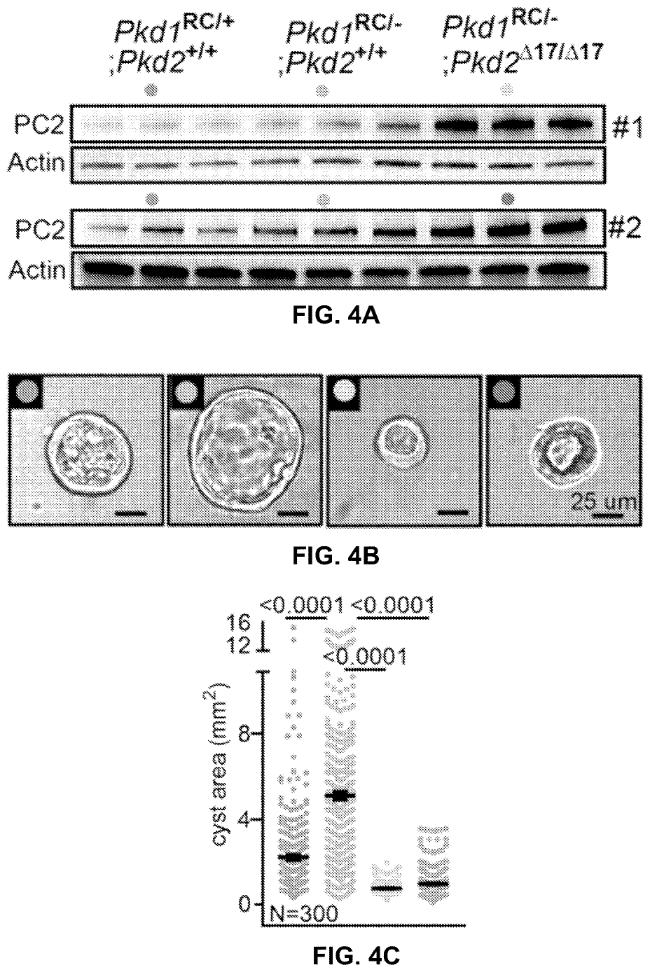

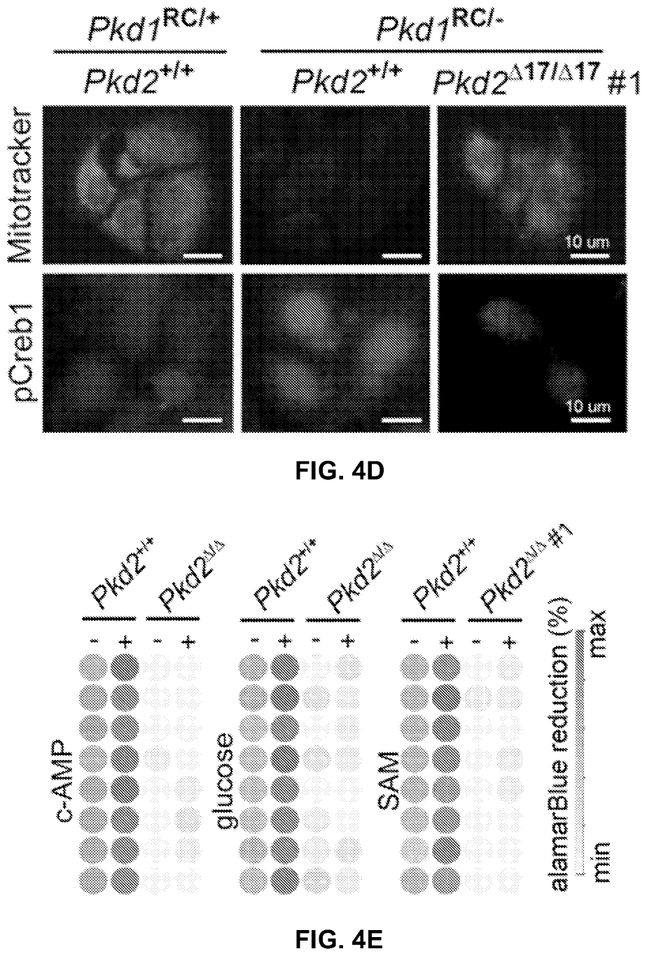

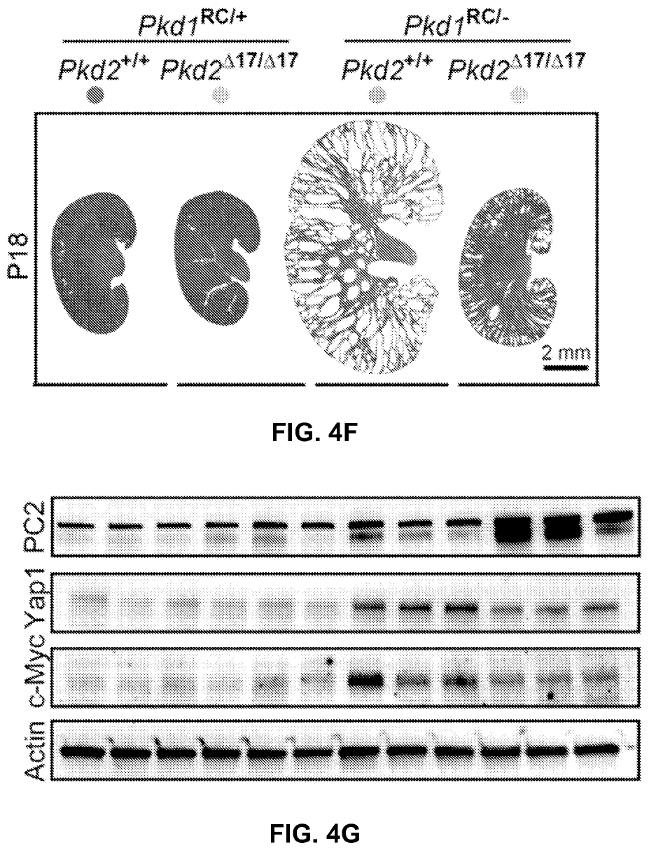

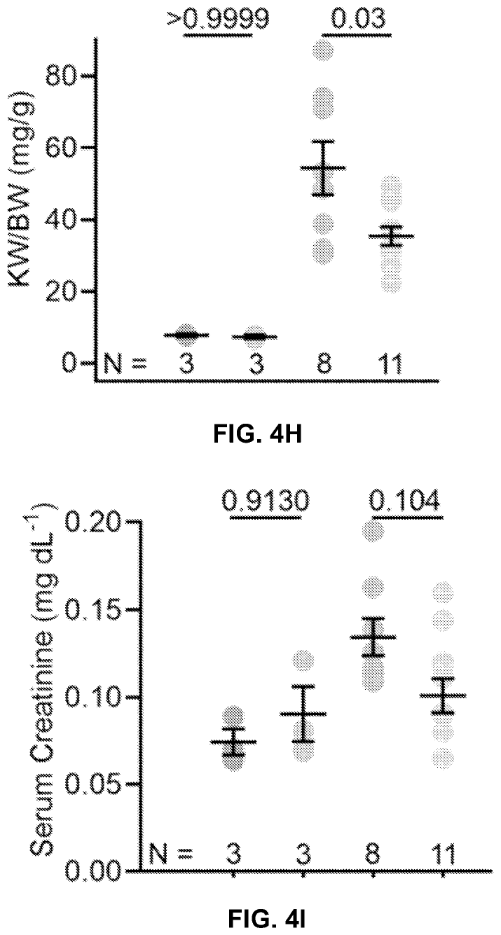

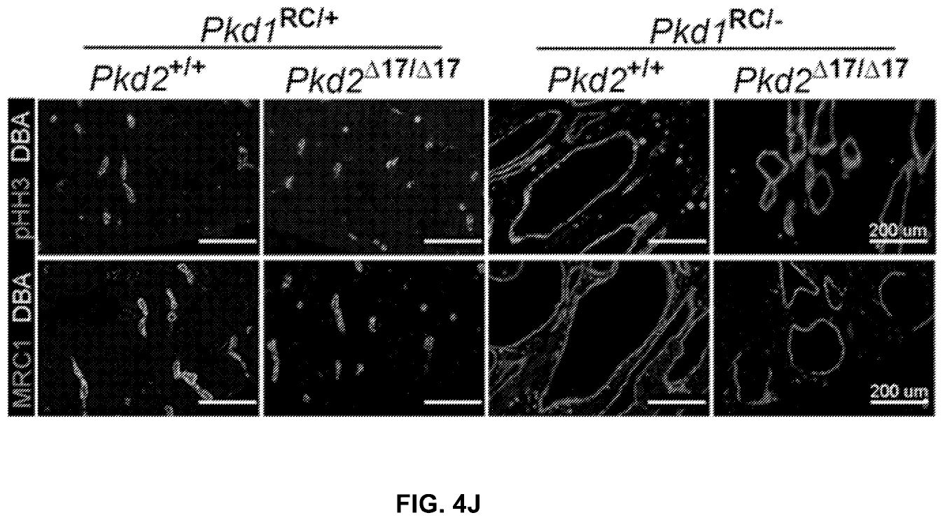

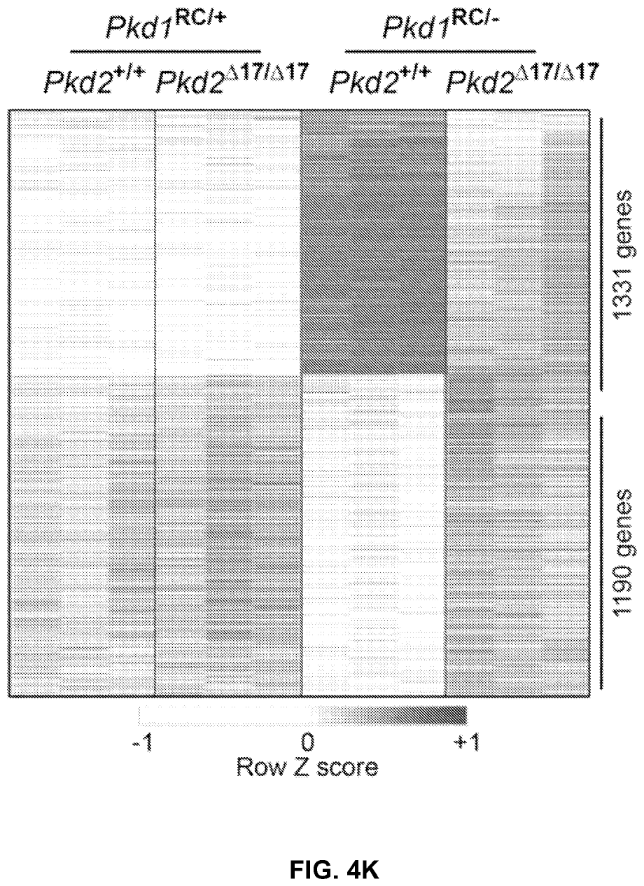

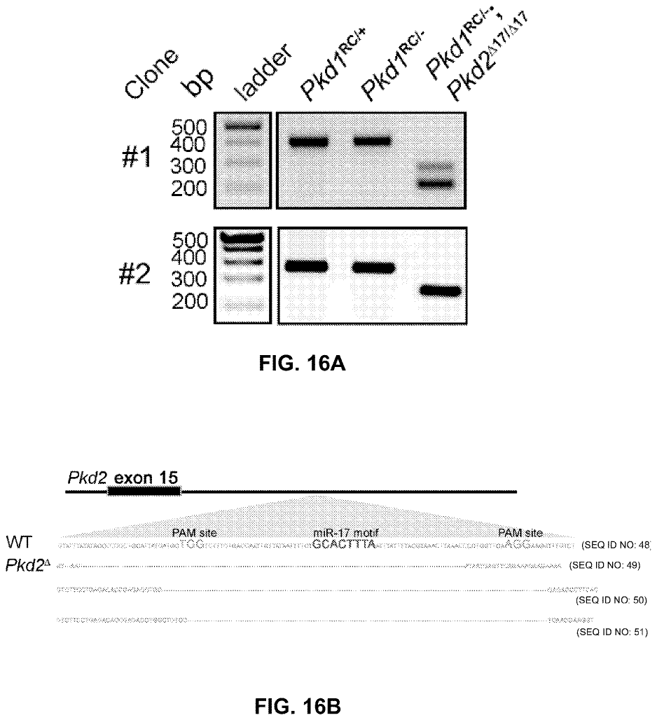

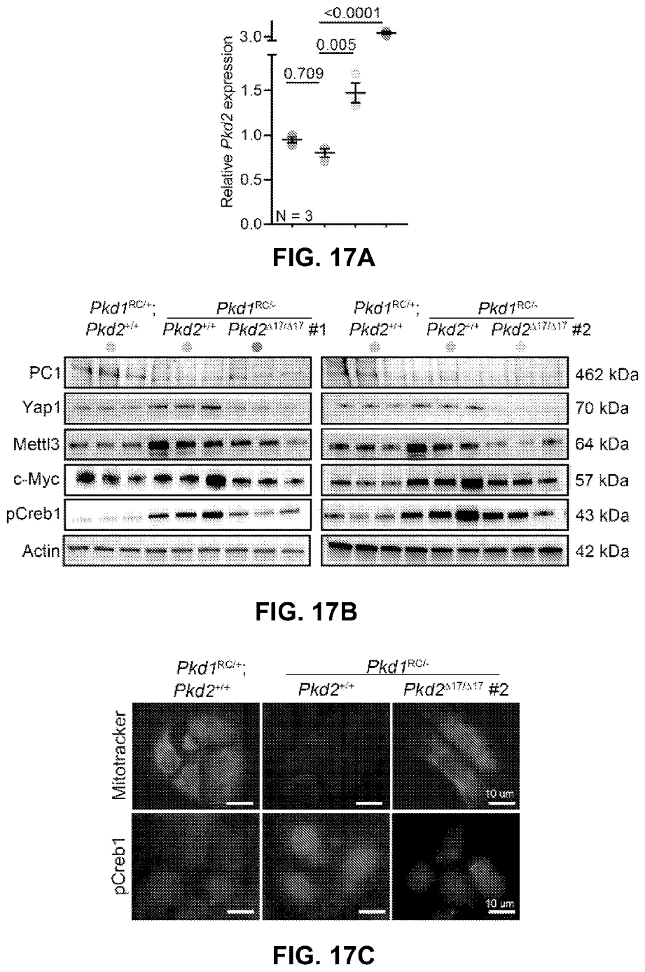

In this example, whether Pkd2 is cis-inhibited and whether preventing this autoinhibition can positively impacts disease progression in Pkd1-mutant models was examined. This is important because a long-standing question has been whether increasing PKD2 can compensate for low PKD1. Interestingly, similar to PKD1, PKD2 harbors an evolutionarily conserved 3′-UTR miR-17 motif. Data in previous examples indicates heightened miR-17 repressive activity in Pkd1-mutant ADPKD models. Therefore, to address these questions, beginning with the Pkd1^{RC/-} cellular model, CRISPR/Cas9 was used to delete the miR-17 motifs from the Pkd2 3′-UTR (Pkd1^{RC/-}; Pkd2^{Δ17/Δ17}) while keeping the Pkd1 miR-17 motif intact (FIG. 16A-16B). Consistent with 3′-UTR cis-inhibition, using qRT-PKDR and immunoblot analysis, higher Pkd2 and PKD2 expression was observed in two Pkd1^{RC/-}; Pkd2^{Δ17/Δ17} clonal cell lines compared to their unedited parental Pkd1^{RC/-} cells (FIG. 4A). PKD1 expression remained unchanged between the edited and unedited cells, indicating the specificity of miR-17 motif deletion from Pkd2 3′-UTRs (FIG. 17B). Surprisingly, it was noted that PKD2 derepression was associated with reduced 3D cyst growth, restored MitoTracker signal, and downregulation of PKDreb1, Yap1, Mettl3, and c-Myc expression in Pkd1^{RC/-}; Pkd2^{Δ17/Δ17} cells compared to Pkd1^{RC/-} cells (FIG. 4B-FIG. 4D and FIG. 17B-17C). As was the case with the Pkd1 3′-UTR deletions, while CAMP, glucose, and SAM promoted proliferation of Pkd1^{RC/-} cells, it was noted that this stimulatory effect was lost in Pkd1^{RC/-}; Pkd2^{Δ17/Δ17} cells (FIG. 4E, Table 4, below).

TABLE 4
alamarBlue assay on Pkd1^{RC/-} and Pkd1^{RC/-}; Pkd2^{Δ17/Δ17} #1 cells.

| | Cell line | | | | | | | | | | | |
|---|---|---|---|---|---|---|---|---|---|---|---|---|
| | | | Pkd1^{RC/-} | | | | Pkd1^{RC/-} | | | | Pkd1^{RC/-} | |
| | Pkd1^{RC/-} | | Pkd2^{Δ17/Δ17} #1 | | Pkd1^{RC/-} | | Pkd2^{Δ17/Δ17} #1 | | Pkd1^{RC/-} | | Pkd2^{Δ17/Δ17} #1 | |
| | Stressor | | | | | | | | | | | |
| | Ctl | cAMP | Ctl | cAMP | Ctl | Gluc | Ctl | Gluc | Ctl | SAM | Ctl | SAM |
| Replicate | | | | | | | | | | | | |
| 1 | 32.32 | 36.36 | 18.7 | 17.73 | 27.94 | 34.09 | 23.44 | 21.66 | 38.11 | 50.04 | 29.66 | 31.93 |
| 2 | 32.14 | 36.15 | 17.51 | 18.6 | 28.8 | 34.05 | 21.61 | 21.57 | 43.64 | 51.71 | 29.57 | 30.41 |
| 3 | 30.55 | 36.5 | 17.79 | 19 | 29.57 | 34.49 | 22.48 | 19.47 | 42.4 | 50.53 | 31.2 | 30.42 |
| 4 | 30.43 | 37.4 | 19.85 | 17.84 | 29.49 | 34.34 | 20.42 | 20.87 | 43.66 | 51.62 | 30.76 | 32.56 |
| 5 | 31.35 | 37.14 | 19.25 | 19.67 | 28.05 | 35.09 | 18.43 | 21.22 | 43.51 | 48.1 | 30.62 | 32 |
| 6 | 32.44 | 38.96 | 19.17 | 19.8 | 27.4 | 35.16 | 19.93 | 21.22 | 43.83 | 53.31 | 30.21 | 32.06 |
| 7 | 32.28 | 36.35 | 19.06 | 21.36 | 28.29 | 35.84 | 20.94 | 18.87 | 42.98 | 49.38 | 30.31 | 30.7 |
| 8 | 32.55 | 38.71 | 18.34 | 17.82 | 27.86 | 36.1 | 19.45 | 20.13 | 43.6 | 48.67 | 33.71 | 30.95 |
| P value | | | | | | | | | | | | |

| | | | |
|---|---|---|---|
| Ctl vs | <0.0001 | <0.0001 | <0.0001 |
| Stressor | | | |

| | | | |
|---|---|---|---|
| Ctl vs | .9519 | .9811 | .8421 |
| Stressor | | | |

| | | | |
|---|---|---|---|
| Ctl vs Ctl | <0.0001 | <0.0001 | <0.0001 |

Raw values of percentage reduction are shown for each biological replicate. The results of one-way Anova followed by post-hoc Tukey's multiple comparisons test are listed.

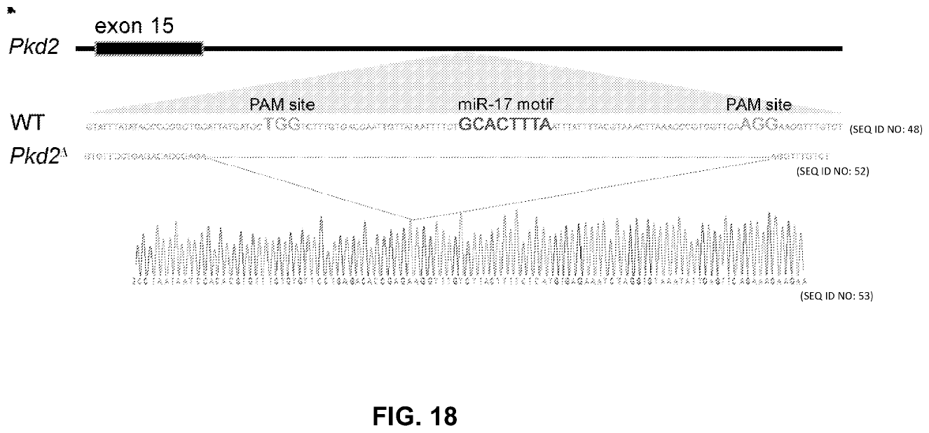

The observations in Pkd1^{RC/-} cells suggest that preventing Pkd2 cis-inhibition and improving PKD2 expression could compensate and retard disease progression in Pkd1-mutant models. This was tested in vivo by deleting the Pkd2 3′-UTR miR-17 motif (Pkd2^{Δ17/Δ17}) in Pkd1^{RC/-} mice. Briefly, CRISPR/Cas9-edited KsPKD^{re}; Pkd1^{RC/RC} mice were used to generate KsPKD^{re}; Pkd1^{RC/RC}; Pkd2^{Δ17/+} mice (see FIG. 18A-18B for details). These mice were then bred with Pkd1^{F/F} mice to eventually generate the following four genotypes: (i) Pkd1^{RC/F}; Pkd2^{+/+}, (ii) Pkd1^{RC/F}; Pkd2^{Δ17/Δ17}, (iii) KsPKD^{re}; Pkd1^{RC/F}; Pkd2^{+/+}, and (iv) KsPKD^{re}; Pkd1^{RC/F}; Pkd2^{Δ17/Δ17}. Characterization of these mice revealed that Pkd2 miR-17 motif deletion in the noncystic setting did not cause PKD2 upregulation, and both Pkd1^{RC/F}; Pkd2^{+/+} and Pkd1^{RC/F}; Pkd2^{Δ17/Δ17} mice exhibited normal kidney histology and function (FIG. 4F and FIG. 4G). In contrast, Pkd2 miR-17 motif deletion in cystic Pkd1^{RC/-} mice was associated with higher PKD2 expression. Moreover, KW/BW and serum creatinine levels were reduced by 34.8% and 25%, respectively, in Pkd1^{RC/-}; Pkd2^{Δ17/Δ17} compared to Pkd1^{RC/-}; Pkd2^{+/+} mice (FIG. 4H-FIG. 4I). Consistently, it was observed that compared to Pkd1^{RC/-}; Pkd2^{+/+} kidneys, Pkd1^{RC/-}; Pkd2^{Δ17/Δ17} kidneys exhibited reduced c-Myc and Yap1 expression (FIG. 4G) and lower cyst proliferation and interstitial inflammation (FIG. 4J). As an additional phenotypic characterization, RNA-seq analysis was performed to compare the kidney transcriptomic profile in the four groups of mice (FIG. 4K). The mRNA expression patterns were nearly identical in Pkd1^{RC/F}; Pkd2^{+/+} and Pkd1^{RC/F}; Pkd2^{Δ17/Δ17}, further implying that Pkd2 miR-17 motif elimination has minimal impact in noncystic kidneys. The cystic Pkd1^{RC/-}; Pkd2^{+/+} kidneys exhibited widespread mRNA dysregulation compared to noncystic Pkd1^{RC/+}; Pkd2^{+/+} control kidneys. It was found that Pkd2 miR-17 motif deletion was associated with improved expression of nearly 50% of these dysregulated mRNAs in Pkd1^{RC/-}; Pkd2^{Δ17/Δ17} (FIG. 4K).

### Example 5: Acute Blockade of Pkd1 and Pkd2 Cis-Inhibition Ameliorates PKD

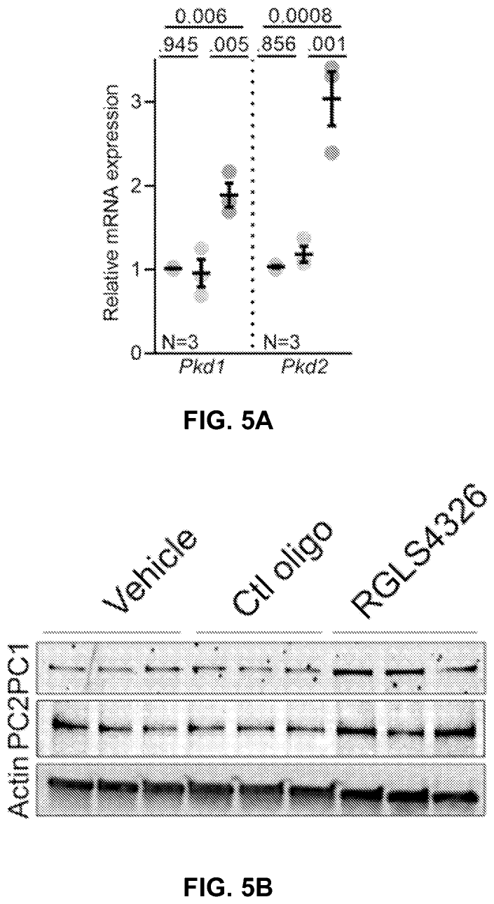

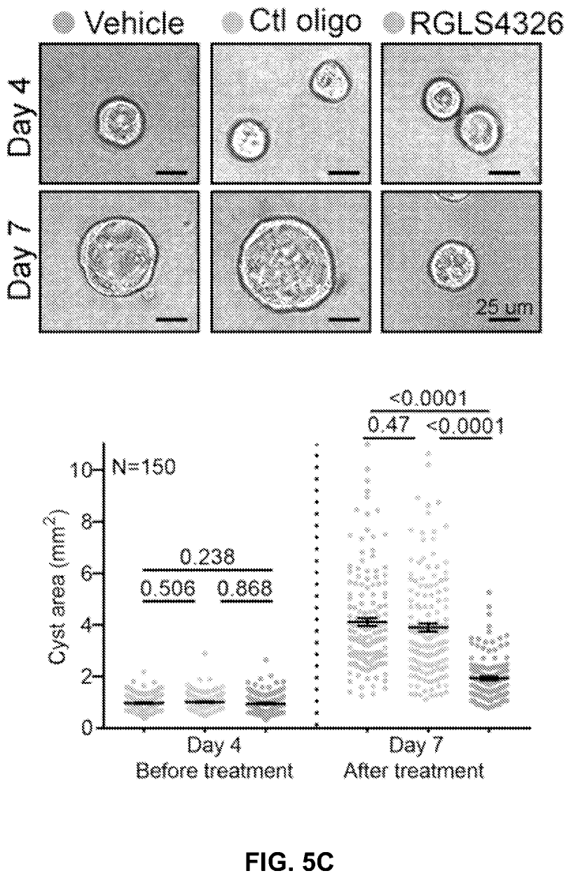

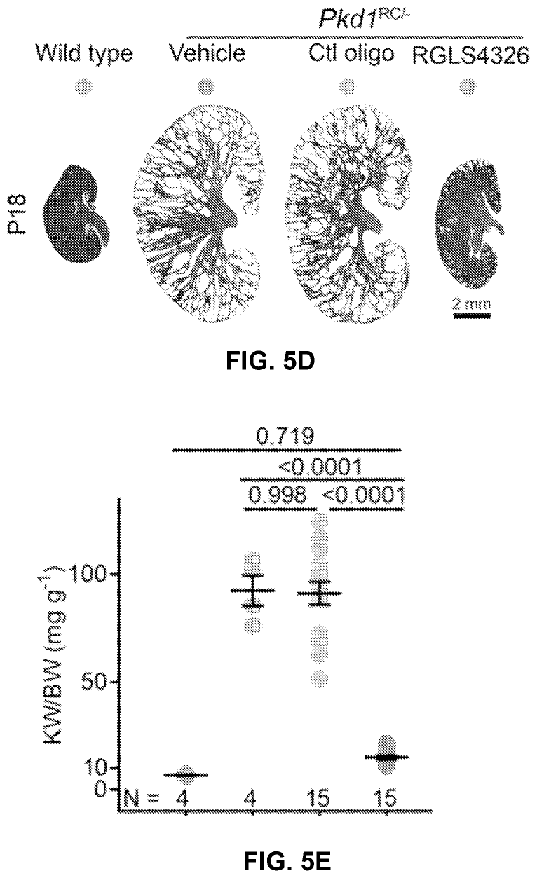

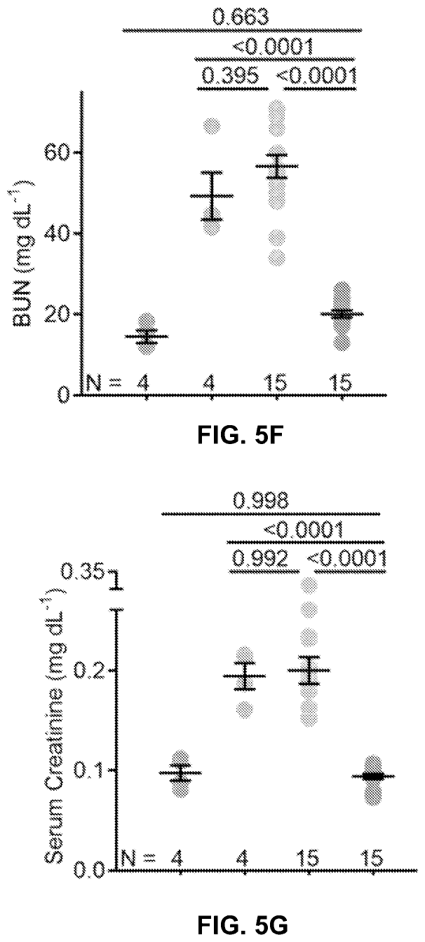

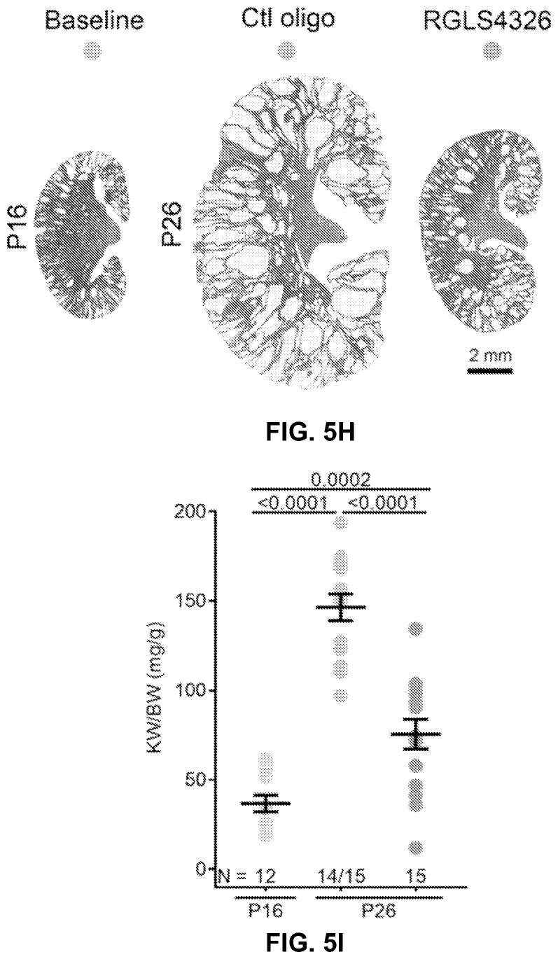

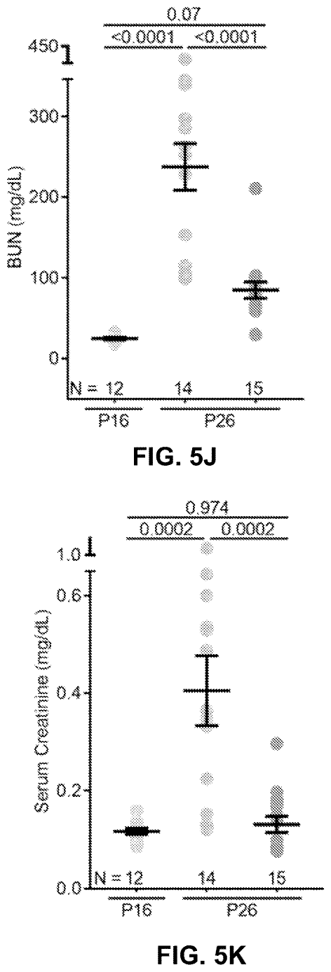

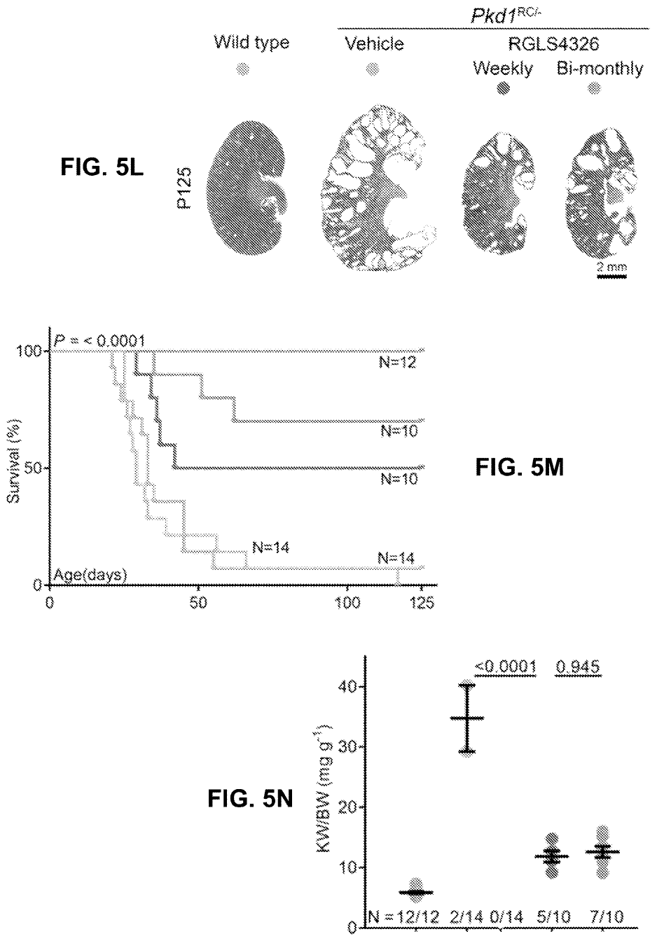

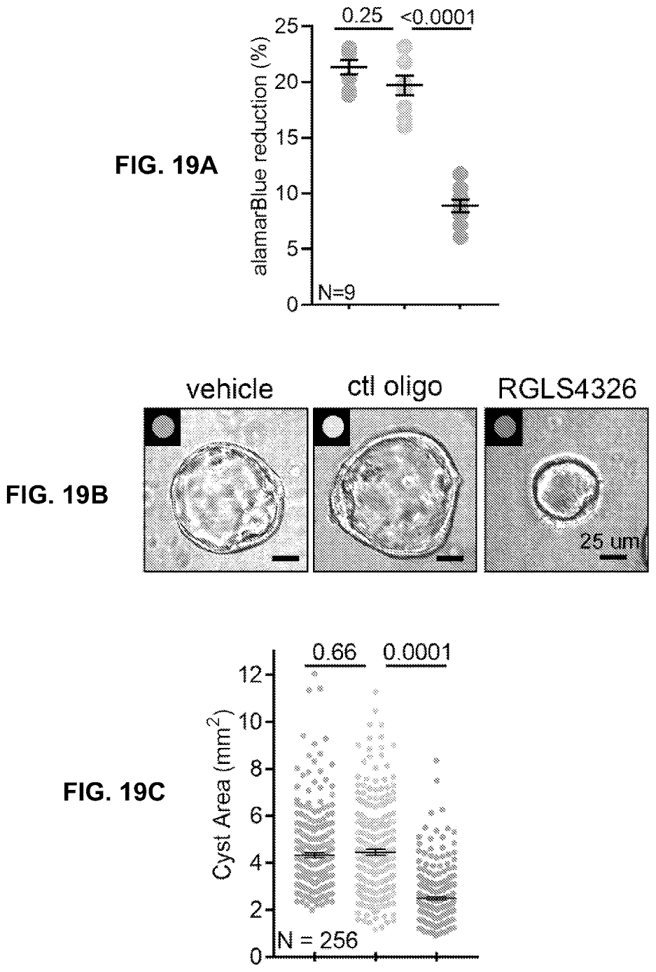

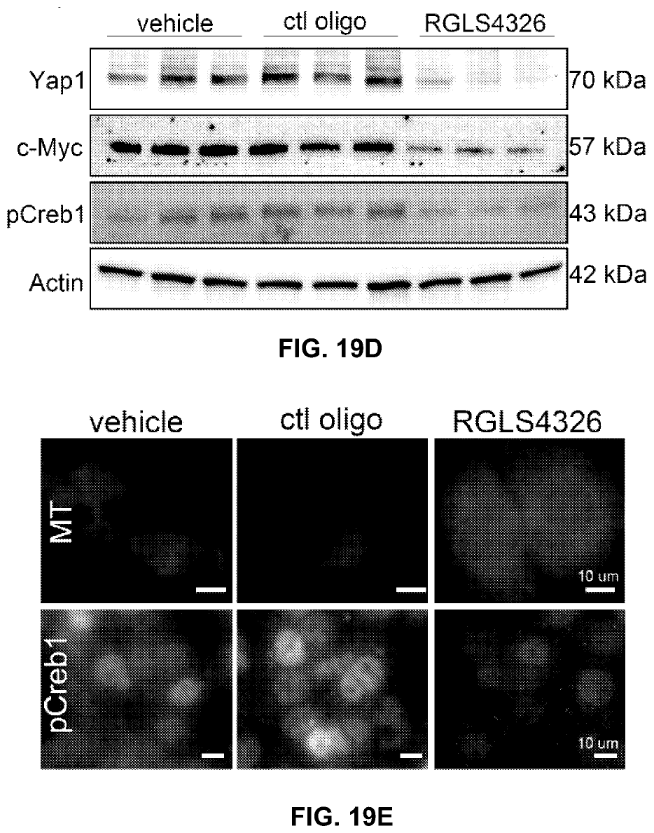

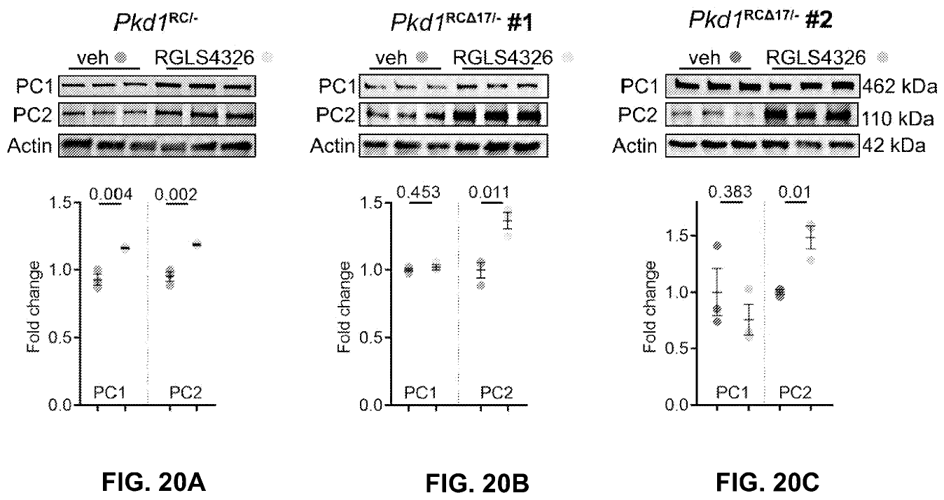

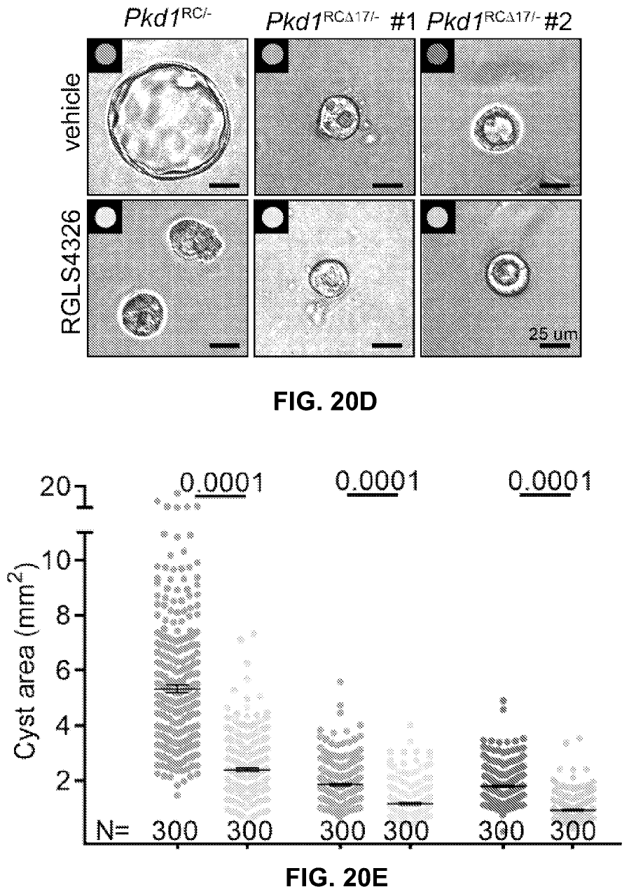

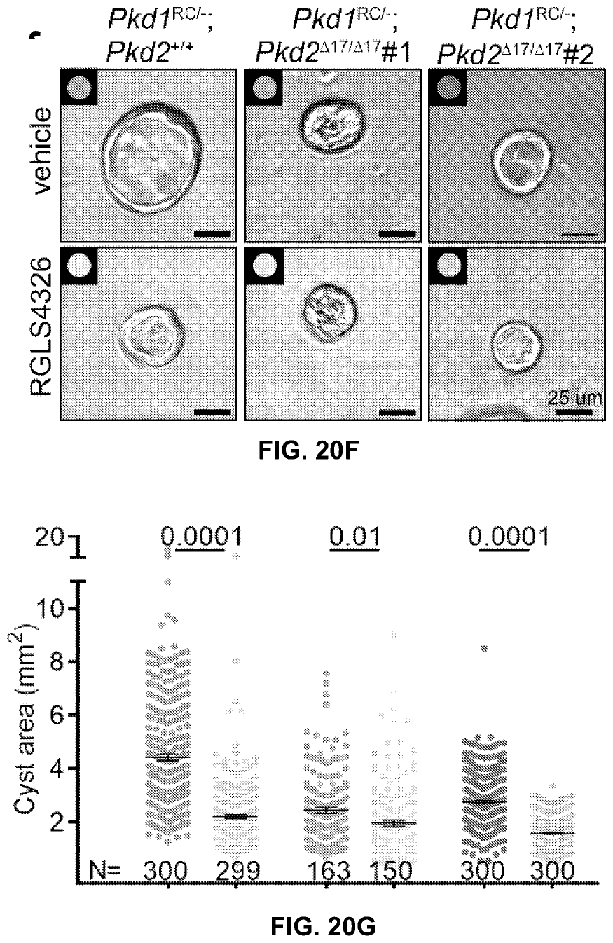

This example provides data showing that acute blockade of Pkd1 and Pkd2 cis-inhibition can ameliorate PKD. This is significant because the data shown in previous examples using CRISPR-edited clonal cellular or mouse ADPKD models both lead to chronic Pkd1 or Pkd2 derepression and are unable to explain whether acute derepression of Pkd1/2, just as the cysts are forming, will prevent disease onset or if restoring Pkd1/2 can alleviate established PKD. To answer these questions, the anti-miR-17 oligonucleotide RGLS4326 was used as a tool to acutely block Pkd1 and Pkd2 cis-inhibition (Lee, E. C. et al. Discovery and preclinical evaluation of anti-miR-17 oligonucleotide RGLS4326 for the treatment of polycystic kidney disease. Nat Commun 10, 4148, doi: 10.1038/s41467-019-11918-y (2019), incorporated herein by reference in its entirety). First, it was confirmed that compared to vehicle (PBS) or control oligonucleotide, RGLS4326 increased Pkd1/2 and PKD1/2 expression in Pkd1^{RC/-} cells (FIG. 5A-5B). The Pkd1/2-boosting effect of RGLS4326 was apparent within three days after treatment. Importantly, RGLS4326 treatment did not lead to higher PKD1 and PKD2 levels in Pkd1^{RCΔ17/-} and Pkd1^{RC/-}, Pkd2^{Δ17/Δ17} cell lines, respectively, confirming that the upregulation of polycystins by this oligonucleotide relies on the miR-17 motif in Pkd1/2 3′-UTRs (FIG. 20B-20C). RGLS4326-treated Pkd1^{RC/-} cells had reduced proliferation, produced smaller cysts in 3D Matrigel cultures, exhibited lower Yap1, c-Myc, and PKDreb1 expression, and a higher MitoTracker signal compared to PBS- or control oligonucleotide-treated Pkd1^{RC/-} cells (FIG. 19A-19E). The cyst-reducing effect of RGLS4326 was present but blunted in Pkd1^{RCΔ17/-} or Pkd1^{RC/-}; Pkd2^{Δ17/Δ17} cell lines, suggesting that this compound mediates its benefits in Pkd1^{RC/-} cells primarily via Pkd1/2 derepression (FIG. 20D-20G). Next, the impact of acutely raising Pkd1/2 in Pkd1^{RC/-} cells after cysts had already formed was tested. Untreated Pkd1^{RC/-} cells were cultured in Matrigel for four days, allowing the cyst to grow. Then these cysts were treated with a vehicle, control oligonucleotide, or RGLS4326, and monitored for three additional days. Vehicle and control oligonucleotide-treated cysts nearly tripled in size, whereas RGLS4326 treatment suppressed this growth (FIG. 5C).

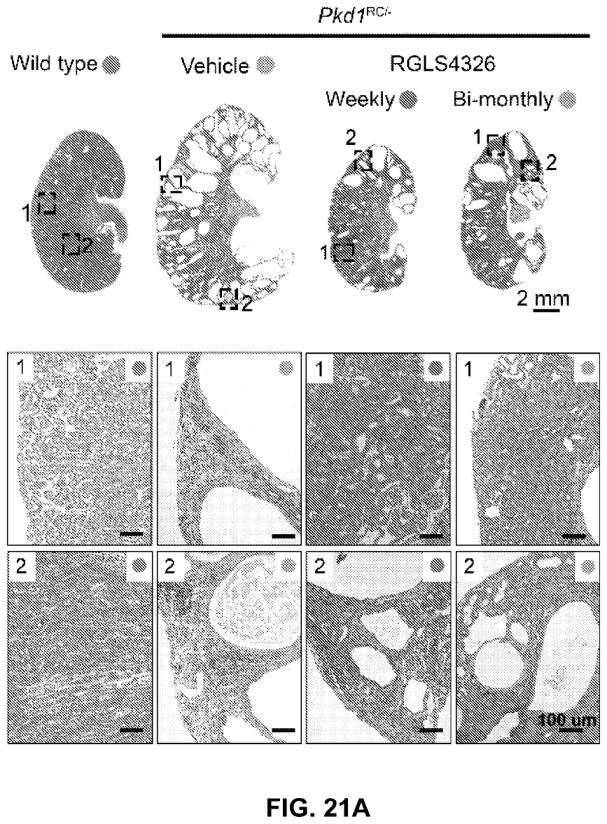

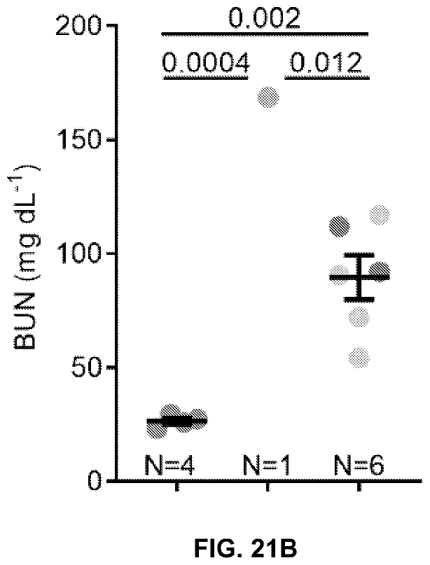

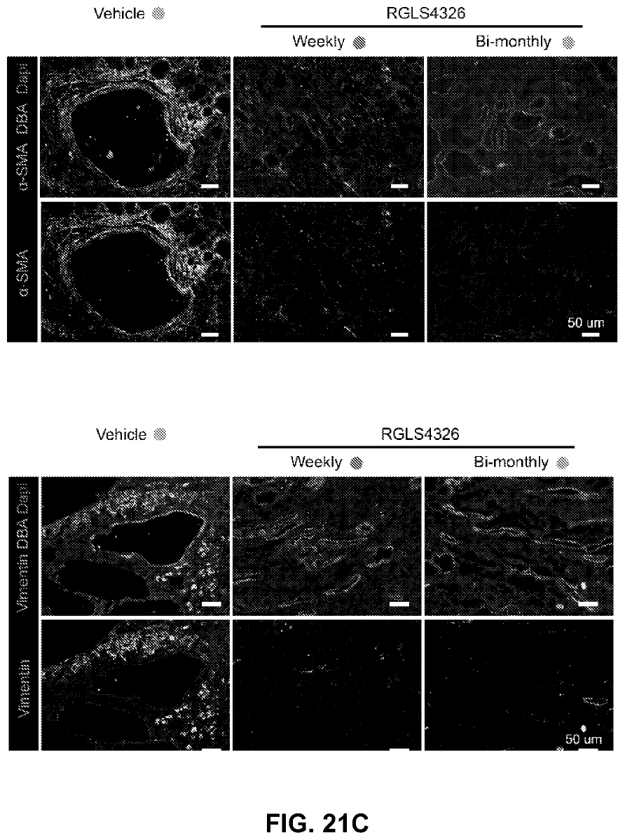

Then it was determined whether the observations in cells can be replicated in vivo. In the first study, Pkd1^{RC/-} mice were treated with vehicle (PBS), control oligonucleotide, or RGLS4326 starting at P10, the age at which cysts begin to form in this model. By P18, marked kidney enlargement was noted with a >10-fold higher KW/BW ratio and elevated BUN and serum creatinine in PBS and control oligonucleotide-treated mice compared to age-matched wildtype mice (FIG. 5D-5G). Strikingly, PKD was virtually prevented, and renal function remained normal in P18 RGLS4326-treated Pkd1^{RC/-} mice (FIG. 5D-FIG. 5G). In the second study, treatment began at P16 when Pkd1^{RC/-} mice had already developed cystic disease. By P26, one out of 15 control oligonucleotide-treated mice had died, and the surviving mice had developed progressive kidney enlargement and near-fatal kidney failure. In contrast, attenuation of PKD progression and stabilization of kidney function was observed in RGLS4326-treated Pkd1^{RC/-}-KO mice (FIG. 5H-5K). Finally, in a third study, long-term effects of Pkd1/2 derepression was assessed in mice that had already developed PKD. Pkd1^{RC/-} mice were treated on P16 and P17 with the vehicle, 20 mg/kg RGLS4326, or 20 mg/kg control oligonucleotide. These mice then received their respective treatment regimens every week until 18 weeks of age. A fourth group of Pkd1^{RC/-} mice received 20 mg/kg RGLS4326 treatment on P16 and P17 and every other week thereafter. 85.7% (12 out of 14) of PBS-treated and 100% (14 out of 14) of control oligonucleotide-treated Pkd1^{RC/-}-KO mice succumbed to their disease before 18 weeks of age. In contrast, 70% (7 out of 10) and 50% (5 out of 10) of Pkd1^{RC/-} mice treated with RGLS4326 bi-monthly or weekly, respectively, survived until 18 weeks of age (FIG. 5M). Furthermore, the surviving mice in the RGLS4326 group had substantially preserved kidney parenchyma (FIG. 5L and FIG. 21A-21C) and reduced KW/BW (FIG. 5N). Thus, acute pharmaceutical Pkd1/2 derepression, including after cyst onset, attenuates murine PKD.

### Example 6: PKD1^{Δ17} or PKD2^{Δ17} Alleles Reduce Cyst Growth of Patient Derived Primary ADPKD Cultures

This example provides data showing that PKD1/2 cis-inhibition is a feature of human ADPKD and that de-repression of this motif in human cells reduces cyst growth in primary ADPKD cultures. Cells derived from cysts of freshly discarded ADPKD nephrectomy samples from four affected individuals (three males aged 41, 48, and 52 years and one 57-year-old female) were used in this example. PKD1 and PKD2 mutation analysis was performed using DNA from cyst cells (Table 5, below). Genomic DNA from ADPKD donor cyst cells was used to perform PKD1 and PKD2 mutation analysis. DNA sequencing and mutation analysis was performed by Ambry Genetics. The details of identified mutations in the four donor cell lines are shown.

TABLE 5
Mutation analysis on kidney cyst
cells of ADPKD donors.

| Donor | Gene | cDNA | Amino Acid | Exon | Type |
|---|---|---|---|---|---|
| 1 | PKD1 | c.6657_6671dup | p.R2220_ | 15 | Insertion |
| | | GCGGCTGGCGCTGC | P2224dup | | |
| | | C | | | |
| 2 | PKD1 | c.8755G > A | p.G2919R | 23 | Missense |
| | PKD2 | c.2418C > T | p.R807* | 13 | Truncating |
| 3 | PKD1 | c.11775dupG | p.R3926Afs*34 | 43 | Truncating |
| 4 | PKD1 | c.7921C > T | p.Q2641* | 21 | Truncating |

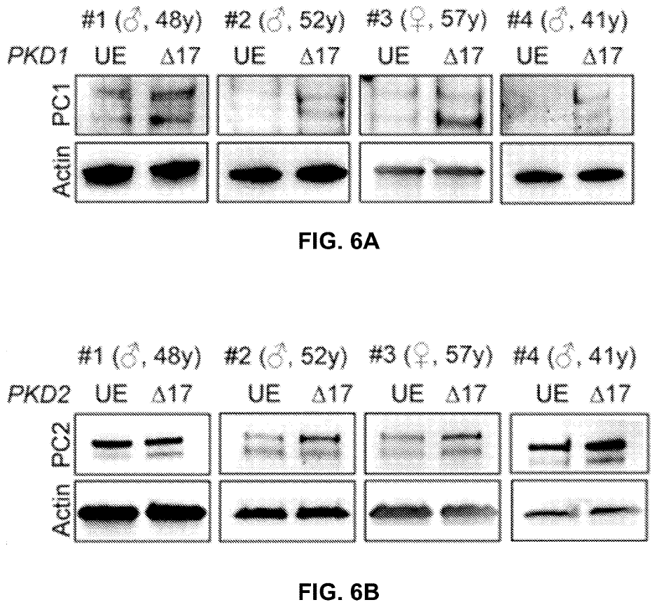

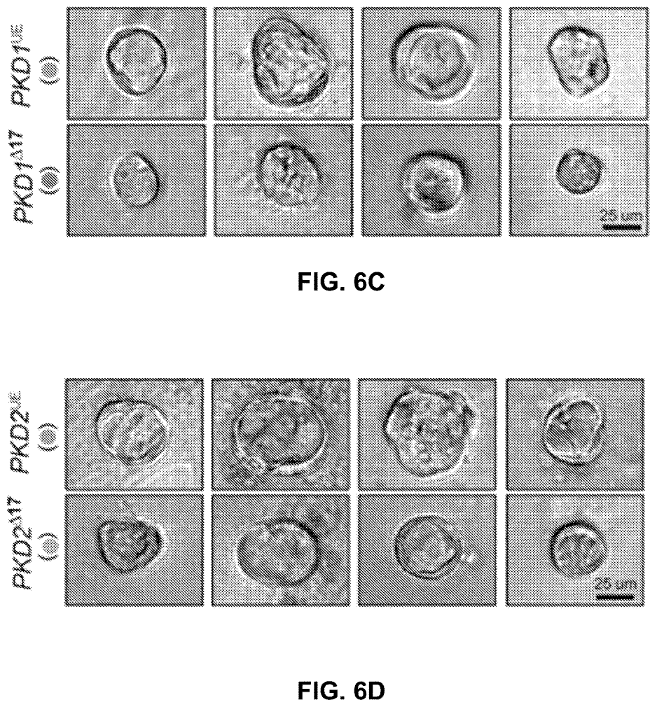

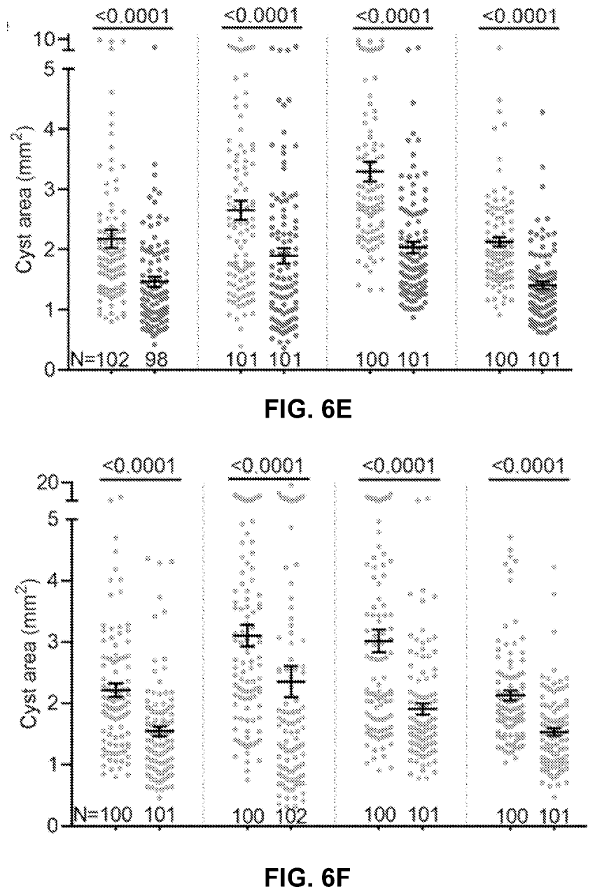

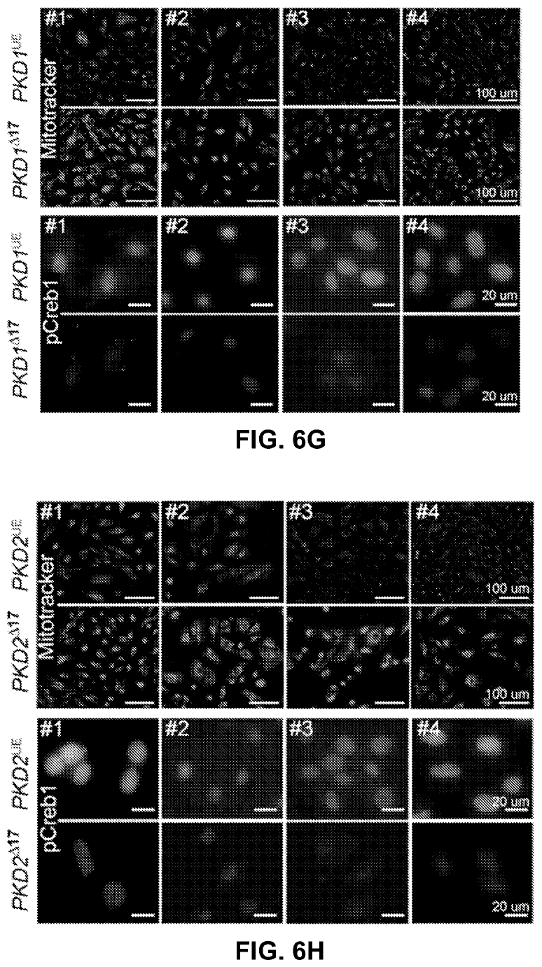

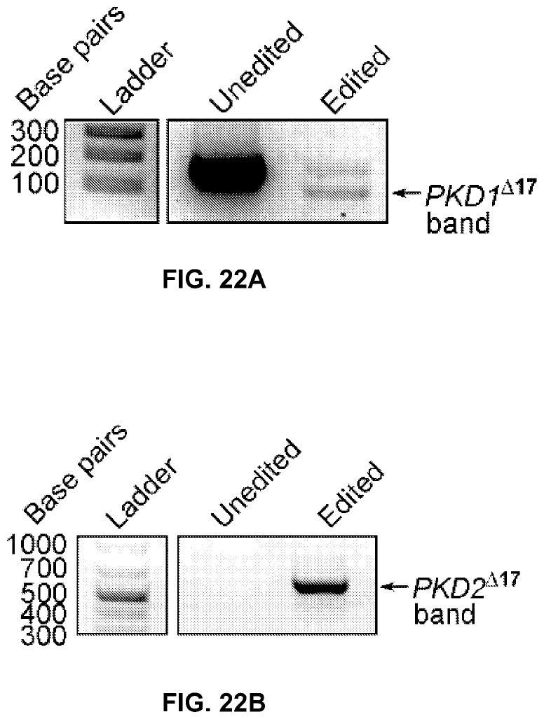

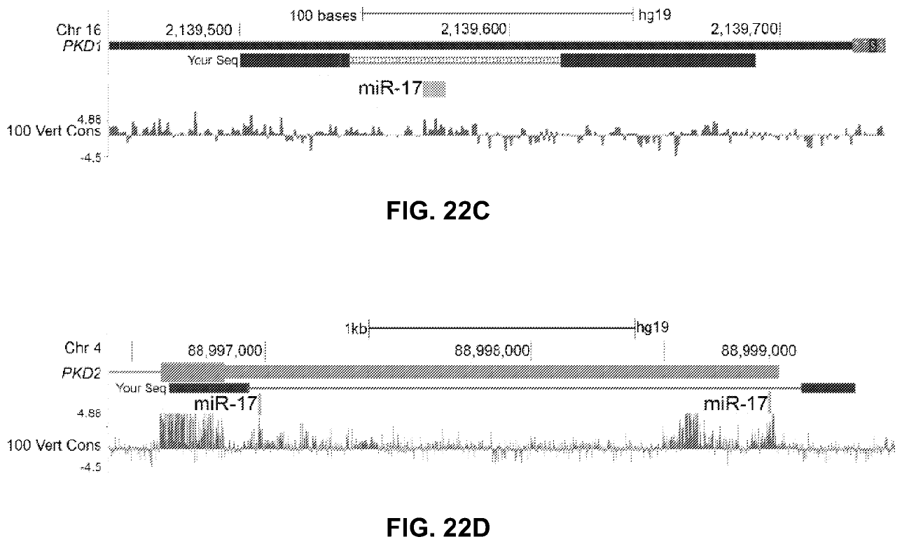

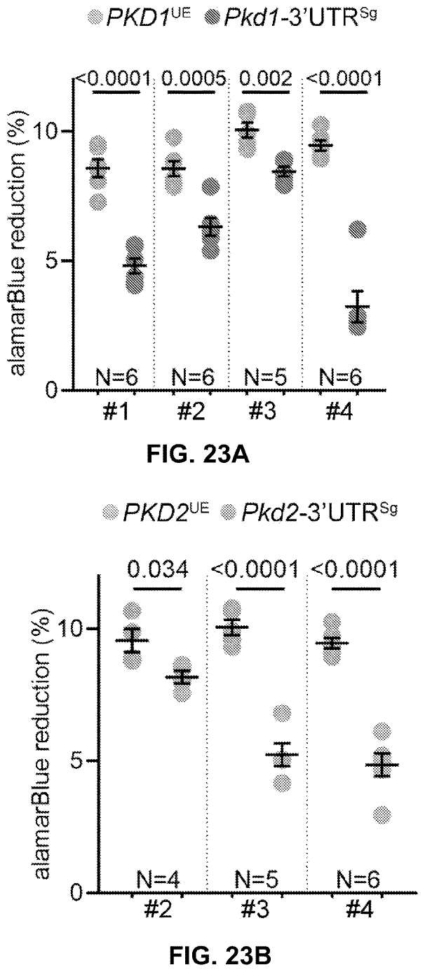

Cell lines #1, #3, and #4 harbor heterozygous PKD1 mutations, whereas cell line #2 harbors heterozygous truncating PKD2 mutation and a missense heterozygous PKD1 mutation. Mutation analysis of cell line #4 was not possible due to technical issues. To assess the translational potential of our findings in mice, CRISPR/Cas9 editing was used to eliminate the PKD1 or PKD2 miR-17 motif in these primary ADPKD cultures (FIG. 22A-22D). Human-specific sgRNAs were designed to target the miR-17 motifs in the PKD1 or PKD2 3′-UTRs. Then primary ADPKD cultures from all four donors were transfected with Cas9 and either PKD1 or PKD2 3′-UTRs sgRNAs. Mock-transfected cells from each donor served as unedited parental controls. Higher PKD1 levels within three days of modeling the PKD1^{Δ17} allele were observed in all four CRISPR-transfected cultures compared to their respective mock-transfected parental controls (FIG. 6A). Similarly, modeling PKD2^{Δ17} alleles led to higher PKD2 expression in CRISPR-transfected cultures than their respective mock-transfected parental controls (FIG. 6B). The functional significance of PKD1 or PKD2 derepression was assessed by performing Matrigel 3D cystogenesis, alamarBlue proliferation assays, live-cell MitoTracker labeling, and anti-PKDREB1 immunofluorescence. The CRISPR-transfected cultures containing PKD1^{Δ17} or PKD2^{Δ17} cells formed smaller cysts (FIG. 6C-6F) and exhibited lower proliferation rates (FIG. 23A-23B), higher MitoTracker signal, and lower PKDREB1 expression compared to their respective mock-transfected, unedited controls (FIG. 6G-6H). These data imply ongoing PKD1/2 cis-inhibition and the potential benefit of derepressing PKD1/2 in human ADPKD cells.

### Example 7—Discussion of Examples 2-6

Examples 2-6 provide a feasible framework for increasing endogenous PKD1 levels and show for the first time that monoallelic Pkd1 derepression is sufficient to alleviate preclinical PKD.

A unifying and parsimonious explanation for ADPKD onset is that cystogenesis ensues when the functional PKD1 dosage falls by 70-80%, dipping below a critical threshold. Thus, germline inactivation of one PKD1 allele alone cannot account for this magnitude of dose reduction. Additional stochastic events that repress the remaining allele are required and play a critical role in determining disease onset. In this regard, data in the examples herein show that miR-17-mediated inefficient translation of mRNAs transcribed by the non-inactivated PKD1 allele represents a targetable, somatic inhibitory ADPKD onset mechanism. As an attractive safety feature, Pkd1 inhibition by miR-17 appears to be an ADPKD-specific phenomenon since it was observed that the miR-17 level is low in normal adult mouse kidneys, and thus, it has no impact on Pkd1 mRNA stability in the non-cystic setting. In contrast, the miR-17 miRNA family becomes activated in PKD models, where it appears to mediate Pkd1 repression well into adulthood, as evidenced by the attenuation of cyst growth by the anti-miR-17 drug RGLS4326, even if the treatment is initiated at later stages of the disease. A noteworthy caveat here is that while RGLS4326 raises PKD1 levels, its benefits in later stages of disease could be derived from simultaneous derepression of other miR-17 targets, including PKD2 and Ppara. This is because RGLS4326 acts on miR-17 itself, rather than on the mRNA target motif. Examples 1-7 herein do not clarify whether targeting the miR-17 cis-inhibition motif directly on the Pkd1 mRNA would be beneficial, but this data is shown in later Examples below. Another insight from this work is that potentially restoring hypomorphic Pkd1 mutants may be a beneficial therapeutic approach. On a cautionary note, particularly for modalities employing exogenous PKD1 supplementation, raising Pkd1 above wildtype levels produces cystic disease in mice. However, the method used in these examples is unique in that, rather than transactivation, it relies on preventing inhibition, making it unlikely that PKD1 will rise to the supratherapeutic range. As a sign that the miR-17-mediated PKD1 inhibition may even be relevant in individuals with ADPKD, it was noted that deleting the PKD1 miR-17 motif in primary human ADPKD cultures increases PKD1, and reduces 3D cyst growth and proliferation. Similarly, inhibiting miR-17 raises PKD1 levels and inhibits the cyst growth and proliferation of primary human ADPKD cultures.

The previous examples also clarify the role of PKD1 in the continual expansion and growth of kidney cysts. Along with cyst initiation, PKD1 inhibition unleashes large-scale transcriptomic and metabolic dysregulation and activates numerous oncogenic pathways, such as cAMP and c-Myc/Yap. In turn, this downstream cyst-pathogenic signaling is thought to fuel cyst expansion. Despite such widespread dysregulation, a recent elegant study reported that transgenic Pkd1 or Pkd2 reconstitution rapidly reverts established cystic disease in mice. Consistently, it was found that acute Pkd1/2 derepression reigns in established cystic disease and makes Pkd1-mutant cells resistant to pro-cytogenic stimuli such as CAMP and SAM. These observations collectively point to PKD1 as the primary, if not the sole, factor governing cyst onset and growth.

These examples also show the unexpected finding that Pkd2 influences the cystic phenotype of Pkd1-mutant models. PKD1 and PKD2 physically interact and are coexpressed at multiple subcellular locations, indicating that the two proteins function in the same physiological pathway. We add a new dimension by extending this relationship into the pathological context. Perhaps, enhancing Pkd2 expression in Pkd1-mutant cells may improve PKD1 trafficking and/or form more heteromeric PKD1-PKD2 protein complexes.

Finally, these examples provides new insights into miRNA biology. miRNAs are well known to simultaneously but subtly repress large mRNA networks. Our approach decouples and disentangles this pleiotropy in the context of PKD. A system was designed where miR-17 is prevented from binding to Pkd1 (or Pkd2) while its ability to interact with its other mRNA targets remains intact. Strikingly, eliminating just one 3′-UTR miR-17 motif phenocopies the effects of inhibiting all of miR-17 in Pkd1^{RC/-} models. Thus, this work is among the first to show that, in some circumstances, the majority of the biological effect of a miRNA can be derived through the repression of a handful of its targets. There are some parallels between this work and the miR-122-Hepatitis C (HCV) infection axis in terms of targeting the disease-central RNA. However, the miR-122 mechanism is unconventional because it targets the foreign HCV RNA genome, binds the 5′-UTR, and aids in HCV accumulation.

Most miRNAs are dispensable for homeostatic tissue functions and are pharmaceutically inhibited with relative ease. Despite these favorable characteristics, miRNA-based drug development has languished compared to other forms of RNA therapeutics. This is partly because the pleiotropic molecular mechanism of numerous downstream mRNA targets makes it difficult to validate the miRNA biological effect or develop pharmacodynamic readouts of anti-miRNA drugs. These examples make the point that prioritizing miRNAs that function as tonic inhibitors of a handful of disease-central mRNAs is likely to be a fruitful drug development strategy. Importantly, these insights are transferable, and it can be speculated that similar modes of therapeutically targetable cis-inhibitory regulation exist in other disorders, especially haploinsufficient monogenetic conditions.

### Example 8—Methods and Materials for Examples 1-7

### Generation of 3′-UTR Cell Lines Via CRISPR/Cas9:

The miR-17 binding site was deleted from the Pkd1 or Pkd2 3′-UTR using CRISPR/Cas9. sgRNAs were designed using www.benchling.com and ordered from IDT. The sgRNA pair targeted DNA sequences upstream and downstream of the miR-17 motif in the Pkd1 or Pkd2 genes. sgRNAs were cloned into the CRISPR mammalian expression vector pSPKDas9 (BB)-2A-GFP as described in Ran, F. A. et al. (Genome engineering using the system. CRISPR-Cas9 Nat Protoc 8, 2281-2308, doi: 10.1038/nprot.2013.143 (2013), incorporated herein by reference in its entirety). Using these sgRNA encoding plasmids, the Pkd1^{RCΔ17/-} cell line and the Pkd1^{RC/-}; Pkd2^{Δ17/Δ17} cell line were generated as follows. To generate the Pkd1^{RCΔ17/-} cell line, Pkd1^{RC/-} cells were transfected with 0.6 μg of the SPKDas9-2A-GFP plasmid carrying the upstream or the downstream sgRNA using Lipofectamine 3000. After 72 hours, FACS was performed to select GFP-positive cells with the top 5% intensity. These cells underwent clonal expansion in 96-well plates. Well-formed colonies were screened for the absence of the miR-17 binding site by DNA PKDR of the targeted Pkd1 genomic sequence. Clones with expected deletion bands were confirmed by Sanger sequencing. Two Pkd1^{RCΔ17/-} clonal cell lines with confirmed deletions were further characterized and analyzed along with their parental control cell lines, as shown in FIG. 2A-2J and FIG. 10A-10C). The same strategy and experimental approach was used for generating the two Pkd1^{RC/-}; Pkd2^{Δ17/Δ17} cell lines (FIG. 4A-4K and FIG. 14). The sgRNA sequences and genotyping primers are provided Tables 6-7, below.

TABLE 6
sgRNA Sequences

| | sgRNA sequence (5′-3′) | |
|---|---|---|
| | Upstream | Downstream |
| | (SEQ ID NO) | (SEQ ID NO) |
| Pkd1 | GCCCATATGTGGACA | GTGTGGCCAGCCAGG |
| | GGTA | ACCCA |
| | (SEQ ID NO: 14) | (SEQ ID NO: 15) |
| Pkd2 | CGAACTTATCATTGT | AAACTTAAACCCGTG |
| | CGTAC | GTTCA |
| | (SEQ ID NO: 16) | (SEQ ID NO: 17) |
| PKD1 | ACGTAGGTTCCCCA | TGTGTGGCCAACCAG |
| | GAGAGCA | GACCC |
| | (SEQ ID NO: 18) | (SEQ ID NO: 19) |
| PKD2 | TTATATGCCCTGAC | AATGTTTTOGGACA |
| | CACCAT | TAGAGA |
| | (SEQ ID NO: 20) | (SEQ ID NO: 21) |

TABLE 7
Genotyping Primer Sequences

| | Genotyping Primers (5′-3′) | |
|---|---|---|
| | Forward Primer | Reverse Primer |
| | (SEQ ID NO) | (SEQ ID NO) |
| Pkd1 | CTAGGGGTCTT | CTGAACCTGA |
| | GGCCATTCC | GGACTTGGGG |
| | (SEQ ID NO: 22) | (SEQ ID NO: 23) |
| Pkd2 | GTTCCTGAGAC | GGCAGCAAAT |
| | ACCGAGAGG | CAGCGTACTT |
| | (SEQ ID NO: 24) | (SEQ ID NO: 25) |
| PKD1 | TGAGGACTCGG | CGTGGAGTCG |
| | GGAAATAAA | GAGTGGAC |
| | (SEQ ID NO: 26) | (SEQ ID NO: 27) |
| PKD2 | GGTCGTGACAG | (WT, |
| | TGAAATCCA | (SEQ ID NO: 29)) |
| | (SEQ ID NO: 28) | CAGAAGATTAG |
| | | CAATCGGTCAC |
| | | (Deletion Specific, |
| | | (SEQ ID NO: 30)) |
| | | TGGCAGCCTATGCT |
| | | GTTATG |

### Generation of 3′-UTR Mice Via CRISPR/Cas9:

The following strains of mice were used: (1) for the mouse models shown in FIG. 1, wildtype C57BL/6N female and male mice were used; (2) for the mouse models shown in FIGS. 2 and 4, KsPKD^{re}; Pkd1^{RC/RC} mice maintained on a C57BL/6J background by our laboratory were used. Prepubertal female mice underwent superovulation using a standard hormone regimen. The epididymis was collected from male mice for sperm harvest. After in vitro fertilization, one-cell fertilized eggs were isolated. CRISPR reagents (IDT) were delivered to the cytoplasm via electroporation using a Nepa21 Super Electroporator (NEPAGENE, Ichikawa, Japan). The eggs that survived the electroporation were washed and cultured in fresh M16 media in microdrop cultures. The eggs were then surgically transferred into the oviducts of day 1 pseudopregnant ICR females. At 21 days of age, founder mice were screened for deletion of the miR-17 binding site by genotyping, and confirmation of deletion was performed by Sanger sequencing.

### ADPKD Mouse Models:

KsPKD^{re}, Pkd1^{F/F}, and Pkd1^{RC/RC} mice were used in this study. All mice were maintained on a C57BL/6J background. At prespecified time points, mice were anesthetized using an approved protocol, and blood was obtained via cardiac puncture. The right kidney was weighed to obtain the KW/BW ratio and immediately flash frozen for future molecular analysis. The left kidney was perfused with ice-cold 1×PBS and 4% (wt/vol) paraformaldehyde. The kidney was subsequently paraffin-embedded. All studies used equal numbers of males and females. The UT Southwestern Institutional Animal Care and Use Committee approved all experiments involving animals.

Pkd1^{RC/+} and Pkd1^{RC/-} Cell Lines:

The Pkd1^{RC/+} and Pkd1^{RC/-} are isogenic, collecting duct-derived epithelial cell lines. These cells were generated from the kidneys of a 14-day-old Pkd1^{RC/flox} male mouse. A single-cell suspension was created by mincing the kidney tissue into 1 mm cubes followed by incubation for 40 min in DMEM containing 5% Collagenase (Sigma #C1639, USA) at 37° C. with intermittent agitation. The cells were then incubated with Biotinylated Dolichos Biflorus Agglutinin (DBA, a collecting duct marker) (Vector labs #B-1035) for 1 hour. DBA-positive cells were isolated using a CELLection Biotin binder kit (Invitrogen #11533D). Subsequently, the cells were immortalized using the SV40 T Antigen Cell Immortalization Kit (Alstem #CILV01). One SV40-positive, immortalized Pkd1^{RC/Flox} clone was infected with an adenovirus that expresses Cre recombinase (Vector Biolabs #1779) to delete the floxed allele, thereby generating the Pkd1^{RC/-} cells. Recombination of the floxed allele was confirmed by genotyping. The uninfected parental clone with the genotype Pkd1^{RC/+} (where the ‘+’ is the floxed allele) serves as the control. These cells are maintained in an epithelial culture medium (Dulbecco's modified Eagle's medium/Ham's F-12 medium supplemented with 2% fetal bovine serum, insulin (8.3×10−7m), prostaglandin E1 (7.1×10−8m), selenium (6.8×10-9m), transferrin (6.2×10−8m), triiodothyronine (2×10−9m), dexamethasone (5.09×10−8m), and recombinant γ-interferon (10 units/ml) at 37° C.

Generation of the Pkd1^{+/+} and Pkd1^{-/-} Cell Lines:

Pkd1^{+/+} and Pkd1-1-cells are isogenic, renal tubule-derived epithelial cell lines. These cells were generated from the kidneys of a 12-day-old Pkd1^{F/F} male mouse pup. Kidneys were isolated and minced into 1 mm cubes. The tissue was incubated for 40 min in DMEM containing 5% Collagenase (Sigma #C1639, USA) at 37° C. with intermittent agitation to create a single-cell suspension. The cells were then strained using a 40-micron cell strainer and incubated with Biotinylated Dolichos Biflorus Agglutinin (DBA) (Vector labs #B-1035) for 1 hour. DBA-positive cells were isolated using a CELLection Biotin binder kit (Invitrogen #11533D). Subsequently, the cells were immortalized using the SV40 T Antigen Cell Immortalization Kit (Alstem #CILV01) and cultured through clonal expansion. Clones were screened for SV40 marker by genotyping for SV40, and one clone was selected for further culture. Pkd11-cells were generated by infecting the Pkd1^{F/F} cells with an adenovirus that expresses Cre recombinase (Vector Biolabs #1779) such that the floxed alleles are ablated. Infected cells were cultured through clonal expansion. The clones were genotyped to confirm successful recombination and deletion of both Pkd1 alleles. The parent Pkd1^{F/F} and the Pkd1^{-/-} cells were further characterized through qRT-PKDR and western blot analysis (FIG. 8A-8B). These cells are grown and maintained in an epithelial cell culture medium described in the section above.

### Histology:

Tissue embedding in paraffin and subsequent sectioning were performed using standard protocols by the Histology core at UT Southwestern Medical Center. The tissues were cut into 5 μm sections and stained with hematoxylin-eosin (H&E) for histological analysis. The stained sections were imaged using a slide scanner.

### RNA:

A Qiagen miRNEASY kit was used for total RNA extraction. cDNA was prepared using an Invitrogen First Strand Superscript III cDNA synthesis kit. Q-PKDR was performed using iQ SYBR Green Supermix (Bio-Rad). All samples were loaded in duplicate or triplicate on the CFX Connect™ Real-time PKDR detection system. 18s was used to normalize mRNA expression. The sequences of the primers are shown in Table 8.

TABLE 8
QPCR primer sequences

| | Forward (5′-3′) | Reverse (5′-3′) |
|---|---|---|
| | (SEQ ID NO:) | (SEQ ID NO:) |
| Pkd1 | CTAGACCTGTC | GCAAACACGCC |
| | CCACAACCTA | TTCTTCTAATGT |
| | (SEQ ID NO: 31) | (SEQ ID NO: 32) |
| Pkd2 | GCGTGGTACCC | CACGACAATCA |
| | TCTTGGCAGTT | CAACATCC |
| | (SEQ ID NO: 33) | (SEQ ID NO: 34) |
| Pkd1 | CATATGGGGCT | GAGGCTGGGTA |
| (+ allele) | TCTGCACTT | CTCACTTGG |
| | (SEQ ID NO: 35) | (SEQ ID NO: 36) |
| Pkd1 | ATTGCTGCCTT | CTGGGTACTC |
| (Δ allele) | CCTTACCCC | ACTTGGTCCA |
| | (SEQ ID NO: 37) | (SEQ ID NO: 38) |

### PKD1 and Other Western Blots:

Total protein was isolated from kidneys or cells using a lysis buffer made by mixing T-PER tissue protein extraction reagent (Invitrogen, catalog #78510) with a protease phosphatase inhibitor tablet (Fisher, catalog #PIA32961) according to the manufacturer's instructions. The lysis buffer was prepared and stored as one-time aliquots at −80° C. The aliquots were thawed on ice immediately before protein isolation. Protein concentration was measured using the Bradford Assay reagent. Protein samples were prepared in 4× NuPAGE LDS Sample Buffer with 0.5% b-mercaptoethanol (Sigma, catalog #M6250) for all proteins except for PKD1 and PKD2 and their loading control beta-actin, which were prepared with 0.1 M DTT (Sigma, catalog #D0632). The samples were always freshly prepared before gel electrophoresis. BME samples were boiled for 5 minutes at 98° C. before loading on gels. The DTT samples were incubated at 25° C. for 10 minutes before loading on gels.

For full-length PKD1 detection, the samples were run on the NuPAGE™ 3-8% Tris-Acetate Protein Gel (Invitrogen, EA03785) at 160 V for 1.5 hours on ice. A high molecular weight protein ladder (Invitrogen, catalog #LC5699) was used in each gel to track 460 kDa proteins. Electrophoretically separated proteins were transferred using the Invitrogen transfer system at 200 mAmps for 100 minutes on ice or at 4° C. The samples containing 10 μg of protein were run on mini-PROTEAN SDS-polyacrylamide precast gels to detect other proteins. A standard molecular weight ladder was used in each gel to track protein sizes. The gels were run at 150 V until the dye ran out. The proteins were transferred to a nitrocellulose membrane using the Trans-Blot Semi-Dry Transfer system on the mixed MW program.

After completing the transfer, the membranes were blocked with 5% fat-free milk and probed overnight at 4° C. with primary antibodies. The membranes were washed three times with 1×TBS-Tween the next morning before and after probing for one hour with a secondary antibody. Goat-anti-rabbit or anti-mouse HRP-conjugated IgG was used as the secondary antibody. HRP-conjugated actin antibody (Sigma, catalog #a3854) was used to measure total protein. The blots were developed using the chemiluminescence substrate SuperSignal West Dura, ECL, or Femto from Pierce. The blots were developed using the Bio-Rad digital imager. The protein bands were quantified using Imagelab software from Bio-Rad. Each Western blot was repeated at least three times. Ten micrograms of protein from cells or kidneys were run on gels to detect <150 kDa proteins. 40-60 μg of protein was run on gels to detect heavy molecular weight (462 kDa) full-length PKD1 protein. All the primary antibodies were used at a 1:1000 dilution, except for PKD1 (used at 1:500), and the secondary antibodies were used at a 1:5000 dilution. The following primary antibodies were used: PKD1 (7E12 Santa Cruz, catalog #sc-130554); PKD1 E8-8C3C10 (Baltimore PKD core center), PKD2 (gift from the Baltimore PKD Core); PKDREB (Cell Signaling, catalog #9198); c-Myc (Abcam, catalog #ab185656), YAP1 (Cell Signaling, catalog #4912); Mettl3 (Invitrogen, catalog #MA5-27527).

### Immunofluorescence on Tissue Samples:

Paraffin sections of kidney tissues were used for immunofluorescence staining. Briefly, the slides were deparaffinized by first baking at 60° C. for 1 hour and then washing in Histo-clear (Fisher, HS-2001) three times for 5 minutes each. Next, the slides were re-hydrated through 100%, 95%, and 70% ethanol washes before incubation in 1×PBS. The slides were then subjected to antigen retrieval with sodium citrate. The slides were treated with sodium borohydride to quench autofluorescence for 40 min. The slides were washed in 1×PBS three times and then blocked in 1×PBS+10% goat serum+0.1% BSA (antibody block) for at least 1-2 hours at RT. Sections were incubated with primary antibodies overnight. Primary antibodies were diluted with antibody block at a 1:500 dilution. Slides were washed in 1×PBS three times for 5 minutes each, treated with Alexa Fluor secondary antibodies (diluted using the antibody block to a 1:500 dilution) for 1 hour, and then washed three times for 5 minutes each. The slides were mounted using Vecta Shield containing Dapi. The slides were imaged using the Zeiss Compound Light microscope or the Zeiss Axioscan Z1 slide scanner. The following antibodies were used: DBA (Vector Labs, catalog #B-1035), THP (Biomedical Technologies, catalog #BT-590), LTA (Vector Labs, catalog #B-1325), MRC1 (Abcam, catalog #ab64693), PKDREB1 (Cell Signaling, catalog #9198), and pHH3 (Sigma, catalog #H0412). Processing, immunostaining, and imaging of slides were performed simultaneously within each experiment.

### Immunofluorescence on Cells:

Immunofluorescence staining was performed on cells grown on 8-chambered slides (Fisher, catalog #154534PK). The cells were fixed with 100% ice-cold methanol for 5 minutes at 4° C. The slides were washed with 1×PBS 3 times for 5 minutes. The cells were then blocked in 1×PBS+10% goat serum+0.1% BSA+0.1 M glycine+0.1% Tween 20 (antibody block) for at least 30 minutes at room temperature. Primary antibodies were diluted with antibody block at a 1:100 dilution and added to the slides for 2 hours. Slides were washed in 1×PBS three times for 5 minutes each, treated with Alexa Fluor secondary antibodies (diluted using the antibody block to a 1:500 dilution) for 1 hour, and then washed three times for 5 minutes each. The slides were counterstained in DAPI (Fisher, catalog #ICN15757410) diluted at 1:10000 in distilled water for 10 minutes before imaging under a Zeiss Compound Light microscope. For each experiment, the control and treatment cells or the control and the Δ17 cells were seeded simultaneously on different chambers of the same slide. Processing, immunostaining, and imaging of slides were also performed simultaneously.

### Mito Tracker Analysis:

MitoTracker Red CMXRos (Thermo Fisher, catalog #M7512) was used to analyze the mitochondrial membrane potential in live cells. The lyophilized MitoTracker® product was dissolved in dimethylsulfoxide (DMSO) to a final concentration of 1 mM and stored at −20° C. in small aliquots. Cells grown to 40-70% confluency were washed with sterile PBS and then treated with regular DMEM serum-free media containing 100 nM Mito Tracker for 8 minutes. Immediately thereafter, the media was replaced with regular growth media and imaged under a Zeiss Compound Light microscope. The images were taken at the same exposure time for the samples of the same experiment. The intensity of fluorescence is directly proportional to membrane potential.

### 3D Cystogenesis Assay:

25 μl of 100% Matrigel (Fisher, catalog #354234) was spread onto each well of an 8-chambered slide with precooled 200 μl sterile pipette tips. The plate was then placed in a 37° C. incubator for 30 minutes for the Matrigel to set. In the interim, cells were trypsinized, washed once with PBS, filtered through a 40 μm cell strainer to create a single-cell suspension, and counted. Cells were seeded on the Matrigel-coated slide at a seeding density of 5000 cells/well in a 300 μl volume of growth media containing 2% Matrigel. For each cell line or treatment condition, cells were seeded in triplicate and incubated at 37° C. for 7 days to allow for the growth of 3D cysts in suspension. During this time, the wells were supplemented with growth medium 72 hours after initial placement into Matrigel. On day 7, the chamber slides were imaged on a Leica DMI 3000B light microscope. The images were analyzed using ImageJ software to obtain cyst size measurements. Each assay was repeated at least three times. Measurements from each experiment were combined and analyzed for statistical significance.

### Ex Vivo Organ Culture:

Female mice bearing potential Pkd1^{+/+}; Pkd1^{Δ/+}, and Pkd1^{Δ/Δ} embryos were dissected at embryonic day (E) 13.5 in PBS to harvest the kidneys and tail. The tail of each embryo was used for DNA extraction and subsequent genotyping. The kidneys were set up for culture on Whatman membranes (Sigma, catalog #WHA110409) in an air-medium interface as described²⁸. The kidneys were cultured in basal DMEM (Thermo Fisher, catalog #12500) containing 10% fetal bovine serum (FBS), 2% PenStrep (Invitrogen, catalog #1514022), 5 μg/ml insulin, 5 μg/ml transferrin, 2.8 nM sodium selenite, 25 ng/ml prostaglandin E and 32 μg/ml T3. One kidney was grown in the above media, and the contralateral kidney was grown in 100 μM 8-Br-CAMP (Sigma, catalog #B7880)-supplemented media. Using a second cohort of mice, one kidney was grown in 100 μM 8-Br-c-AMP or 100 UM 8-Br-c-AMP+250 UM SAM. For all the cultures, the media was changed every 48 hours. The cultures were imaged live using the Zeiss Stereo Lumar microscope on day 4. The cysts were measured and analyzed using ImageJ software. At the end of 6 days, the kidneys were flash-frozen and stored at −80° C. until further use for RNA or protein extraction.

alamarBlue Assay:

Pkd1^{RC/-} and Pkd1^{RCΔ17/-} cells (3×10\hat{ }3 density) were seeded on 96-well plates. The next morning, the medium was changed to contain 1× alamarBlue reagent (Invitrogen, catalog #DAL1025) and vehicle, 100 μM 8-Br-CAMP, 100 UM SAM, or 17 mM glucose. Colorimetric readings were taken at 570 nm and 600 nm in a microplate reader after 12 hours. The redox reaction of alamarBlue was used to assess cell proliferation quantitatively. N=8 was used for each condition. The values were plotted as a scaled heatmap using the Python MatplotLib package. The same experimental approach was used for the Pkd1^{RCΔ/-}; Pkd2^{Δ17/Δ17} cells and the control cells Pkd1^{RC/-}; Pkd2^{+/+}

### Serum Electrolytes:

Serum creatinine was measured by capillary electrophoresis by the UT Southwestern O'Brien Center. BUN was measured by Vitros250 Analyzer by the UT Southwestern Metabolic Phenotyping Core.

Microarray Analysis of microRNAs:

Total RNA was extracted from kidneys using miRNeasy mini kits (Qiagen). The small RNA fraction (<300 nucleotides) was hybridized on a μParaflo Microfluidic chip containing detection probes for all mouse microRNAs (miRNAs) in the miRBase version-17 (miRBase, http://microrna.sanger.ac.uk/sequences). The hybridized microarray chips were labeled with fluorescent dyes and laser scanned to obtain fluorescent images. The signal values for each sample were derived by background subtraction and normalization. Microarray chip hybridization, fluorescent labeling, laser scanning, and background subtraction and normalization were performed by LC Sciences. The signal values from each of the five age groups (P2, P7, P14, P35) were averaged, and the P values using One-way ANOVA were calculated. The differentially detected signals are defined by P<0.05.

### RNA-Seq Preprocessing:

Sequencing quality control was performed with FastQC v0.11.8. RNA-seq reads were trimmed, and low-quality reads were removed using Trimgalore v0.6.3_dev (www.bioinformatics.babraham.ac.uk/projects/trim_galore/) with the “paired” parameter and length of 150 bps. Trimmed fastq sequences were aligned to the mouse reference genome GRCm38 using STAR aligner v2.5.3a with the produced bam files sorted by coordinate by using the option “--outSAMtype BAM SortedByCoordinate.” Raw read gene counts were obtained using STAR aligner with the options “-quantMode GeneCounts” and “--sjdbGTFfile” with gene models in GTF format obtained from mouse EnsEMBL release 94. Alignment quality control and read mapping statistics were obtained from Picard tools v2.20.3 using the function “CollectMultipleMetrics” (broadinstitute.github.io/picard/).

### RNA-Seq Data Analysis:

Raw gene counts were used for quality control and differential expression analysis. Raw counts were normalized to the total number of reads by calculating log 2CPM (counts per million). We carefully examined the log 2CPM distribution and its relationship to the standard deviation and determined the appropriate cutoff (average Log₂CPM<−3) to eliminate lowly expressed genes before differential gene expression analysis. TPM (transcript per million) quantification was performed using RSEM v1.3.1, and differential gene expression analysis was performed using the limma-trend (version 3.40.6) in R^{58,59}.

### Transcript Quantification:

Individual Pkd1 transcript quantification was performed using Salmon v1.3.0⁶⁰. The five different transcript versions for Pkd1 were added to the RefSeq mm9 fasta reference transcriptome to build a novel Salmon index. Then, the fastq files were directly mapped and read counts, and TPM values were quantified with the standard process.

### In Vitro RGLS4326 Experiments:

Pkd1^{RC/-} cells were seeded at 2×10 \hat{ }5 confluence in 6-well plates. The next morning, cells were transfected using Lipofectamine 3000 with a vehicle, control oligonucleotide, or RGLS4326 at a final concentration of 100 UM. Forty-eight hours after transfection, the cells were collected for RNA extraction. Seventy-two hours after transfection, cells were harvested for protein or further seeded for alamarBlue assay and 3D cystogenesis assay. For the experiment shown in FIG. 5C, the 3D cystogenesis assay was performed with untreated Pkd1^{RC/-} cells, as described in the methods section of the 3D cystogenesis assay with the following changes. On day 4 of Matrigel culture, the wells were imaged using the Leica light microscope DMI 3000B, and then the cultures were transfected with a vehicle, 100 μM control oligonucleotide, or 100 μM RGLS4326 and grown for 3 additional days. On day 7, the samples underwent imaging to assess cyst size.

### RGLS4326 Mouse Experiments:

The KsPKD^{re}; Pkd1F/RC mouse line was used for the drug studies. Mice were randomly assigned and administered 20 mg kg⁻¹ vehicle (PBS), control oligonucleotide, or RGLS4326 via subcutaneous injections. For the first cyst prevention study (FIG. 5D-5G), mice were injected on postnatal days (P) 10, P11, P12, and P16 and sacrificed on P18. Nontransgenic strain-matched mice were also sacrificed on the same days. For the second disease stabilization study (FIG. 5H-5K), mice were injected at P16 and P17 and sacrificed at P26. One mouse from the study succumbed to the disease and died earlier than 26 days of age. For the third long-term study (FIG. 5L-5N), mice were injected on P16 and P17 and then every week until 18 weeks of age. Another cohort of mice received the same dose of RGLS4326 treatment on P16 and P17 and then semimonthly thereafter until 18 weeks of age. The mice were observed every day for 18 weeks to note death. At the end of 18 weeks, the surviving mice were sacrificed to harvest tissue. Equal numbers of males and females were used in all study groups.

### Human ADPKD Cell Experiments:

Primary human ADPKD cyst cells were obtained from PKD Research Biomarker and Biomaterial Core at the University of Kansas Medical Center (KUMC). The use of surgically discarded kidney tissues complied with federal regulations and was approved by the Institutional Review Board at the University of Kansas Medical Center. PKD1 and PKD2 mutation analysis of DNA from donor cyst cells was performed by Ambry Genetics (Aliso Viejo, CA). Each primary cell line was cultured in DMEM/F12++ (Gibco, catalog #10565-018) supplemented with 10% FBS, 5 μg kg⁻¹ insulin, 5 μg mL⁻¹ transferrin, and 5 ng mL⁻¹ sodium selenite and incubated in an atmosphere of 95% air and 5% CO₂ at 37° C. until 80% confluency. At the 2^{nd} passage, cells from each human donor underwent reverse transfection using CRISPRMAX reagent (Invitrogen) containing Cas9 protein (IDT) and synthetic sgRNAs (IDT) or were transfected with vehicle (lacking Cas9). The Cas9/sgRNA-transfected cultures are a mixed population of edited and unedited cells. Clonal propagation was not possible because these are primary cells that allow only a limited number of passages. Cas9- or vehicle-transfected cells (control) were then seeded into 6-well plates and chamber slides. After 72 hours, the cells were harvested for genotyping, Western blot analysis, and immunofluorescence/MitoTracker staining. In addition, at 72 hours posttransfection, cells were trypsinized and plated at 4000 cells/well density in 130 μl of media plus Matrigel (Corning, catalog #354234) in a 96-well plate (Corning, catalog #353072). Media was replenished 72 hours after the initial placement into Matrigel. Cyst images were obtained on the 7th day of Matrigel culture (10th day after Cas9/SgRNA or vehicle transfection). One hundred cyst images were obtained for Cas9- or vehicle-transfected cells from each donor. Similarly, 72 hours post-transfection, cells were seeded at 2000 cells/well density in 96-well plates for the alamarBlue proliferation assay. The following day, the media was replaced with growth media containing 1× alamarBlue, and readings were taken 12 hours later.

### Statistics and Reproducibility:

All experiments were carried out with at least three biological replicates and showed successful reproducibility. For in vivo experiments, N is the number of mice analyzed. For in vitro experiments, N refers to the number of biological replicates. Two-tailed Student's t-test was used for pairwise comparisons and analysis of variance (ANOVA), followed by Tukey's post hoc test was used for multiple comparisons. The Mantel-Cox test was used for the analysis of mouse survival. All data were analyzed using Prism software (GraphPad Software). P<0.05 was considered statistically significant. The sample size and P values are mentioned in the figure graphs, the figure legends, or the results section. For the RGLS4326 studies, animals were randomly assigned to treatment arms. Investigators were not blinded to the treatment or the genotypes of the animals.

### Data Availability:

The RNA-seq datasets have been deposited in the NCBI Gene Expression Omnibus repository under accession number GSE196237 [www.ncbi.nlm.nih.gov/geo/query/acc.cgi?acc=GSE196237]. The microarray dataset has been deposited in the NCBI Gene Expression Omnibus repository under accession number GSE208429 [www.ncbi.nlm.nih.gov/geo/query/acc.cgi?acc=GSE208429].

### Example 9—Design and Verification of Pkd1 Stabilizing Oligo in Mouse Kidney Epithelial Cell Lines

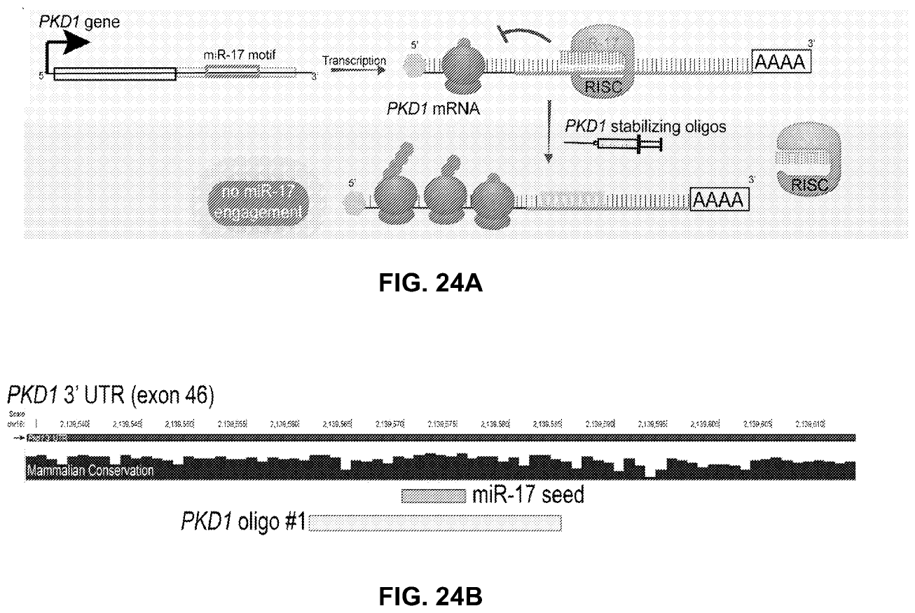

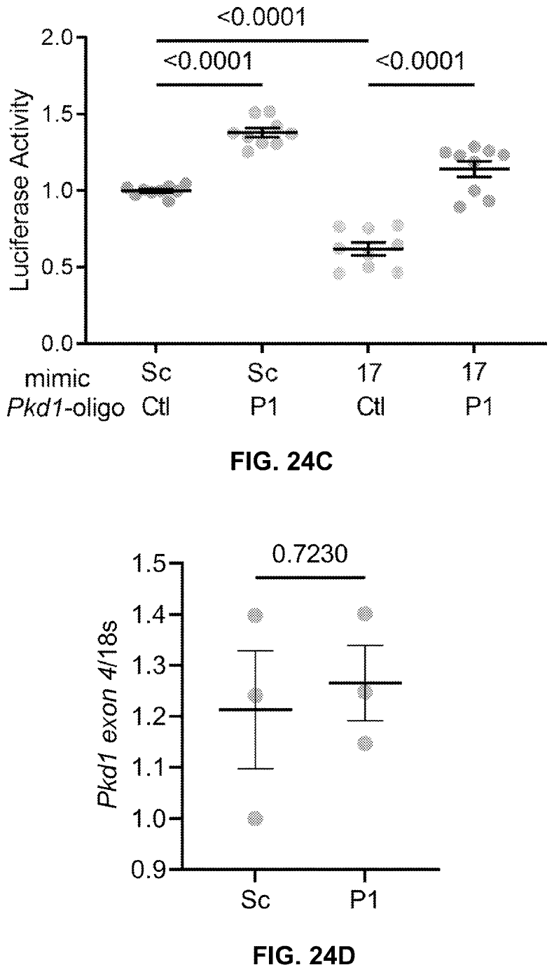

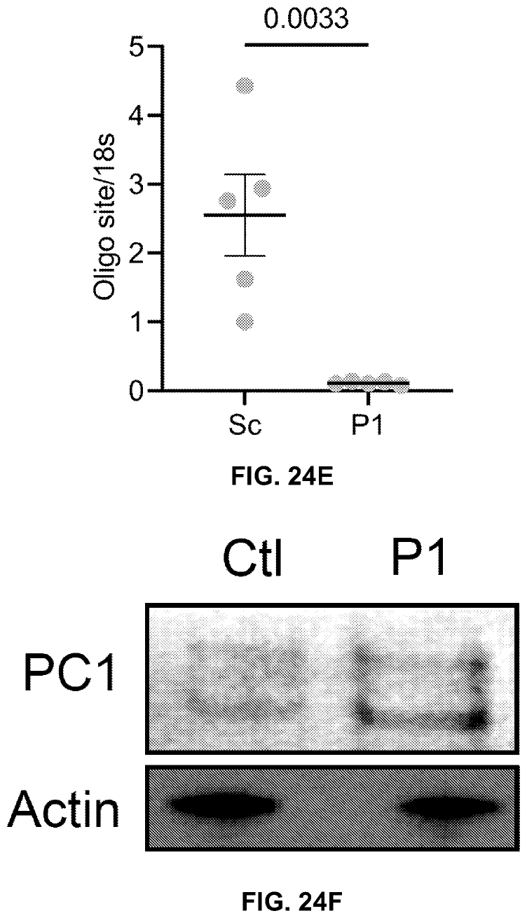

To test whether PKD1 dosage could be modulated by directly targeting miR-17 engagement on Pkd1 mRNA, an PKD1 stabilizing oligo binding to a 3′ untranslated region (UTR) of the PKD1 mRNA was designed. A graphical depiction of the action of these oligos is shown in FIG. 24A and an alignment relative to the 3′UTR is shown in FIG. 24B. Three oligos were generated targeting mouse or human Pkd1 mRNA or mouse/human Pkd2 mRNA as shown in the Table 9 below. The two ASOs targeting mouse or human Pkd1 mRNA are referred herein below as “oligo 1” and “oligo 2”, respectively.

TABLE 9

| | | | SEQ |
|---|---|---|---|
| | | | ID |
| PKD Target | Species | Sequence | NO: |
| PKD1 “oligo 1” | Mouse | TAAAGTGC | 1 |
| | | TGAAGCC | |
| PKD1 “oligo 2” | Human | TTAAAGTG | 2 |
| | | CAGAAGCC | |
| PKD2 | Mouse/ | TAAAGTGC | 3 |
| | Human | AGAAAATT | |

mIMCD3 cells were transfected with pls-PKD1-3′-UTR reporter plasmid, microRNA mimic (sc or miR-17) and scramble (ctl) or Pkd1 oligo (SEQ ID NO: 1) and then levels of luciferase measured 72 hours after transfection as a reporter for PKD1 expression (see Methods in Example 13). FIG. 24C shows that cells treated with Pkd1 oligo (SEQ ID NO: 1) had increased signal and therefore increased PKD1 expression. Meanwhile, qRT-PKDR for Pkd1 mRNA spanning exon 4-5 in scramble and Pkd1 oligo treated mouse kidney epithelial cells demonstrates no change in Pkd1 transcript (FIG. 24D). Further, Pkd1 oligo binding prevents cDNA synthesis over Pkd1-3′-UTR binding site evidenced by lack of Pkd1 3′-UTR transcript detection in Pkd1 oligo treated cells (FIG. 24E). The increase in PKD1 expression was confirmed using western blot showing increased Polycystin 1 (PKD1) in Pkd1 oligo treated kidney epithelial cells (FIG. 24F).

### Example 10—Pkd1-Oligo Stabilizes Pkd1 mRNA and Slows Cyst Growth in a Pkd1 Mutant Cell Line

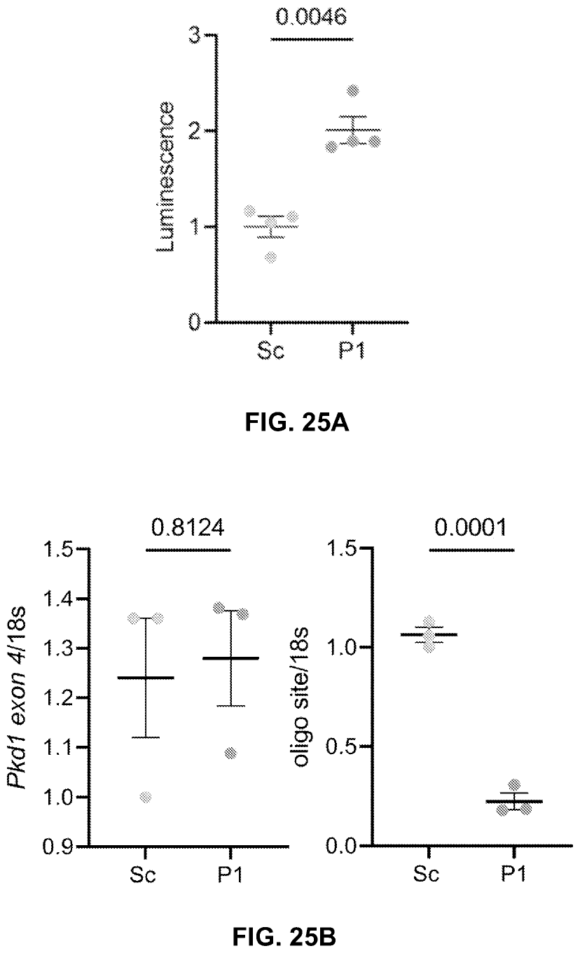

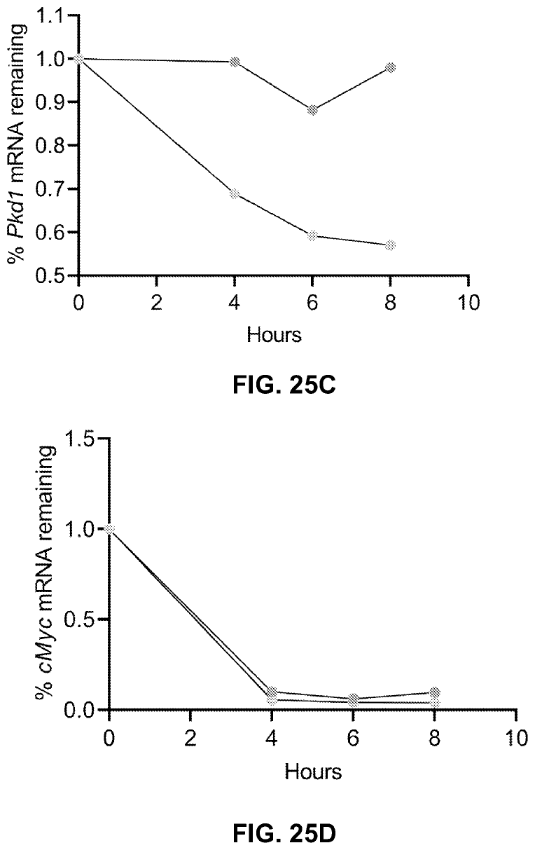

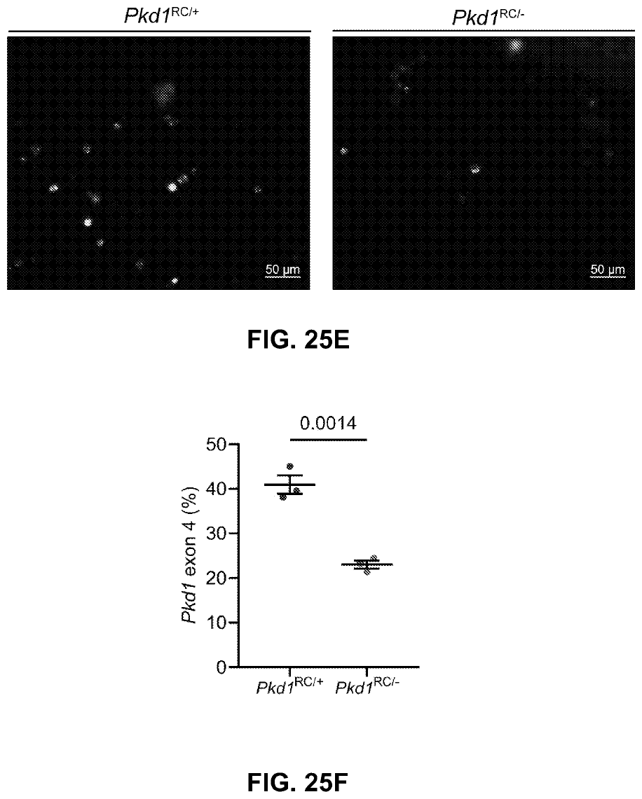

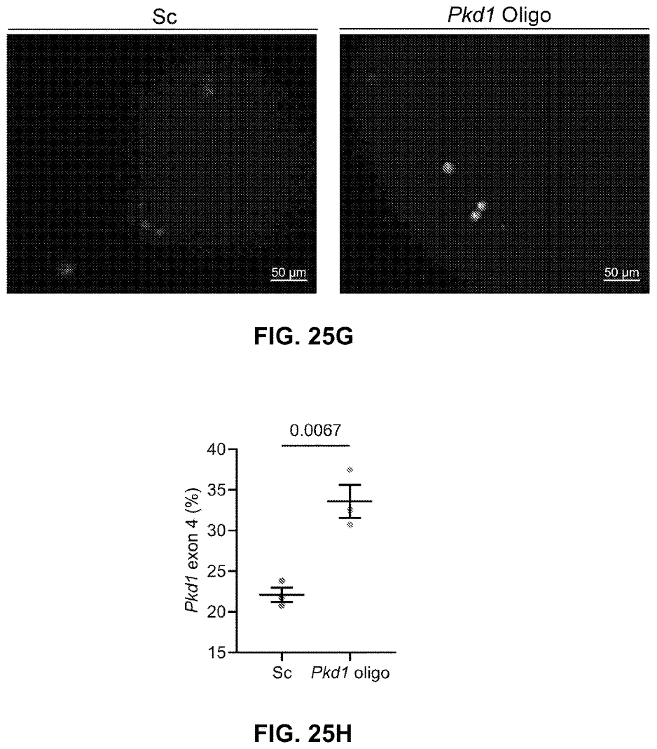

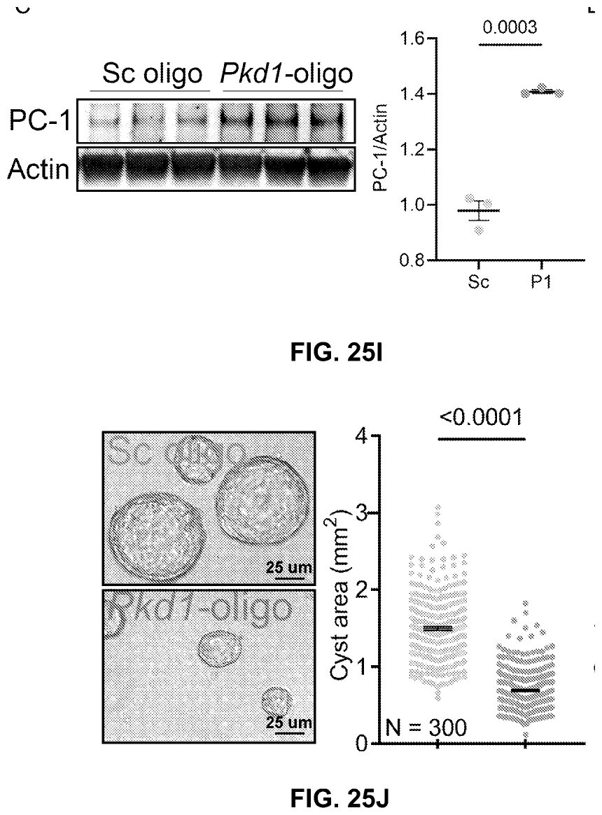

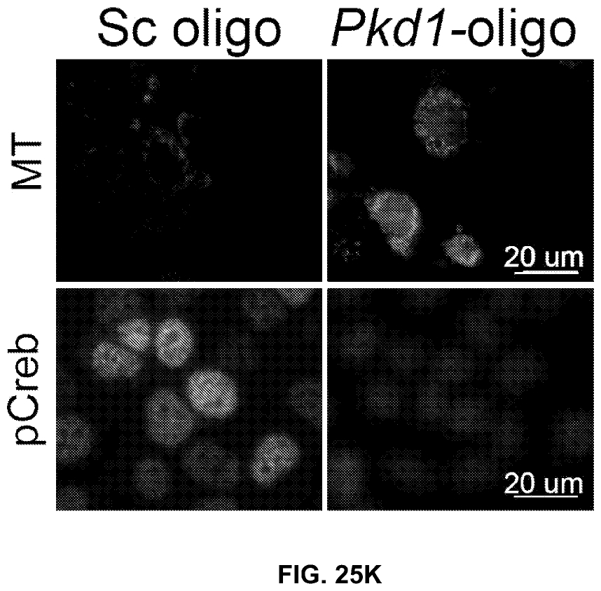

In this example, data is shown demonstrating that Pkd1 oligos can stabilize Pkd1 mRNA and slow cyst growth in model cell lines. First, Pkd1RC/cells were transfected with a Pls-PKD1-3′-UTR reporter plasmid along with a Pkd1 (SEQ ID NO: 1) or scrambled oligo to monitor Pkd1-3′-UTR activity with luminescence. As shown in FIG. 25A, luminescence activity measured after 72 hours showed increased Pkd1-3′-UTR activity in Pkd1 oligo treated cells. In another experiment, Pkd1^{RC/-} cells were transfected with Pkd1 (SEQ ID NO: 1) or scramble oligo and then analyzed with qRT-PKDR for transcripts spanning exons 4-5 and transcripts encompassing the Pkd1 3′-UTR. As shown in FIG. 25B, overall levels of Pkd1 mRNA did not change but levels of Pkd1 3′-UTR transcript were depleted in Pkd1 oligo treated cells. Therefore, Pkd1 oligo binding prevents cDNA synthesis over Pkd1-3′-UTR binding site.

In another experiment, Pkd1^{RC/-} cells transfected with scramble or Pkd1 oligo (SEQ ID NO: 1) were treated with actinomycin to inhibit transcription 48 hours after transfection. Samples were harvested for at 0, 4 and 8 hours to measure abundance of mRNA transcripts. Pkd1 mRNA degradation is inhibited by Pkd1 oligo (FIG. 25C). c-Myc transcript is equivalently degraded in both scramble and Pkd1 oligo treated samples (FIG. 25D).

In another experiment, Pkd1^{RC/+} and Pkd1^{RC/-} cells were plated into 6 well plates (1×10⁵ cells per well) and the next morning transfected with a pDAC565 plasmid (Addgene #195242) which also bears a guide RNA targeting exon 4 of the Pkd1 gene (e.g., CAGCCACGCCAGACCACAGTTGCACTCAAATG (SEQ ID NO: 39)) using Lipofectamine 3000 (Invitrogen) to reach a final concentration of 40 nM. After 48 hours cells were visualized under 40× fluorescence microscopy. Quantification of Pkd1 mRNA abundance was determined by counting number of cells which were transfected with plasmid (indicated by background nuclear green fluorescence) that displayed GFP expressing puncta or cloud like signal (indicative for Pkd1 mRNA). 200 cells with GFP glow were counted per each condition. Each experiment was completed 3 times. FIG. 25E shows representative fluorescent images of treated cells. FIG. 25F shows a graphical plot of the percentage of cells with detected Pkd1 mRNA. It was found that there was a 50% reduction in Pkd1 mRNA detection between Pkd1^{RC/+} and Pkd1^{RC/-} cell (FIG. 25E-25F).

To determine whether Pkd1 oligo could rescue the loss of Pkd1 mRNA levels after knockdown, Pkd1^{RC/-} cells were plated into 6 well plates (1.5×10⁵ cells per well) and the next morning transfected with Scramble or Pkd1 oligo (SEQ ID NO: 1) and pDAC565 plasmid (containing the guide RNA targeting exon 4 of the Pkd1 gene) using Lipofectamine 3000 (Invitrogen) to reach a final concentration of 40 nM. After 48 hours cells were visualized under 40× fluorescence microscopy. Quantification of Pkd1 mRNA abundance was determined by counting number of cells which were transfected with plasmid (indicated by background nuclear green fluorescence) that displayed GFP expressing puncta or cloud like signal (indicative for Pkd1 mRNA). 200 cells with GFP glow were counted per each condition. Each experiment was completed 3 times. FIG. 25G shows representative fluorescent images of treated cells. FIG. 25H shows a graphical plot of the percentage of cells with detected Pkd1 mRNA. There is a significant increase in Pkd1 mRNA detection in cells treated with Pkd1 oligo versus Scramble oligo (FIG. 25G-25H).

Pkd1^{RC/-} cells transfected with scramble or Pkd1 oligo (SEQ ID NO: 1) were then analyzed for PKD-1 expression, cyst size and mitochondrial activity. As shown in FIG. 251, Pkd1 oligo treated Pkd1^{RC/-} cells (pink) showed increased Polycystin 1 protein (PKD-1) expression as compared to Scramble treated cells (purple). Likewise, FIG. 25J shows that Pkd1 oligo treated Pkd1^{RC/-} cells (pink) had reduced cyst size as compared to Scramble treated cells (purple). Finally, Pkd1 oligo treated cells displayed enhanced mitochondrial activity measured by MitoTracker signal (red) and reduced PKDREB (green) expression (FIG. 25K).

### Example 11—Pkd1 Oligo Increases PKD1 Expression in Three Immortalized Human ADPKD Kidney Epithelial Cell Lines

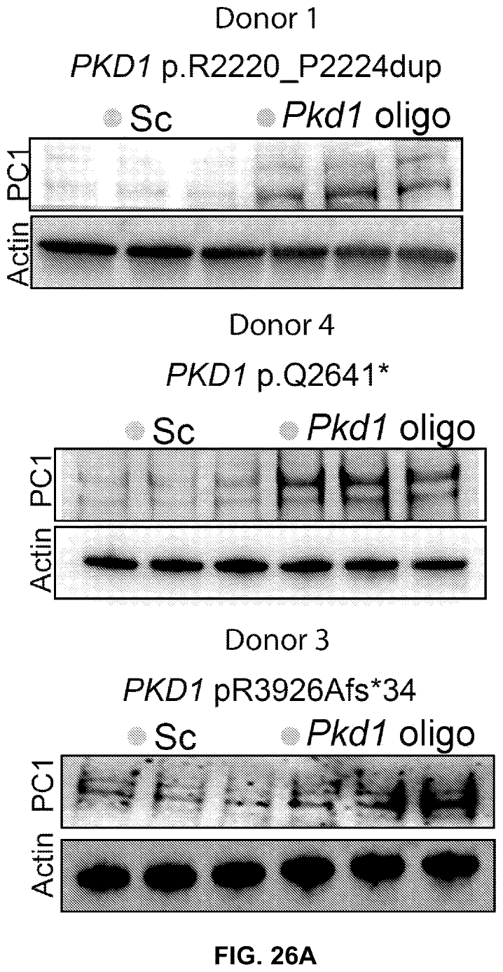

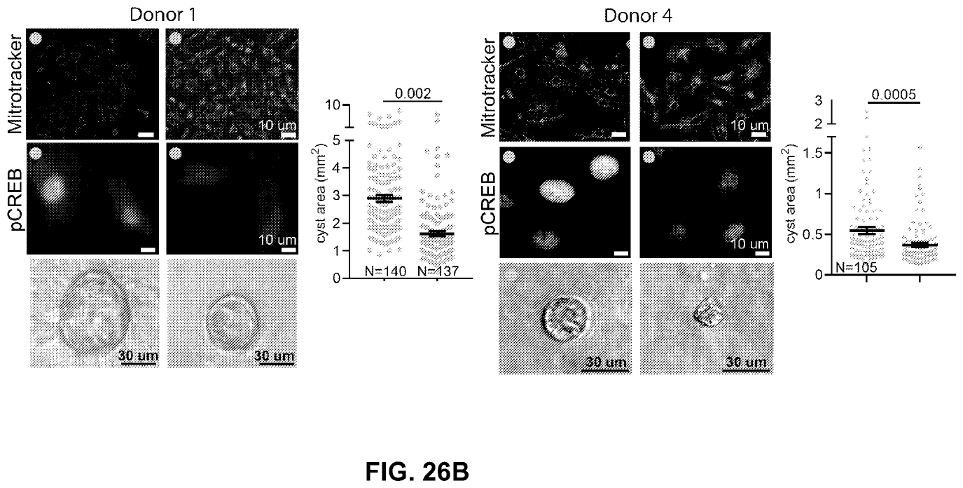

In this example, data is shown that Pkd1 oligo (SEQ ID NO: 1) can increase Pkd1 expression in human cell lines. Specifically, three cell immortalized human ADPKD kidney cell lines (each having one inactivated or mutant PKD1 allele) were treated with the Pkd1 oligo (SEQ ID NO: 1) and then analyzed for PKD-1 protein expression, cyst size, mitochondrial activity and overall gene expression. FIG. 26A depict illustrative western blots demonstrating increased PKD1 expression of the remaining PKD1 allele in all three cell lines. FIG. 26B shows that treated cells also had reduced cyst size, increased MitoTracker signal (red) and reduced PKDREB expression (green).

### Example 12—Side by Side Testing of Pkd1 Oligo 1 and 2 in Mouse and Human ADPKD Kidney Cell Lines

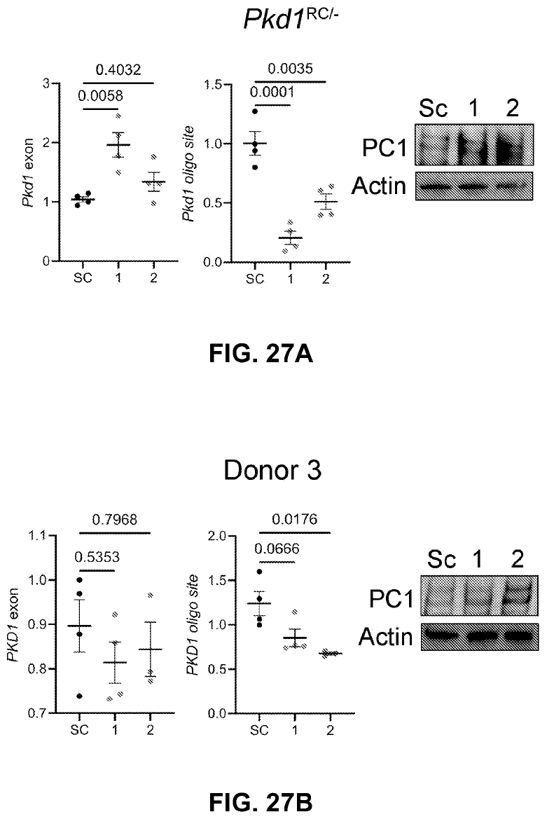

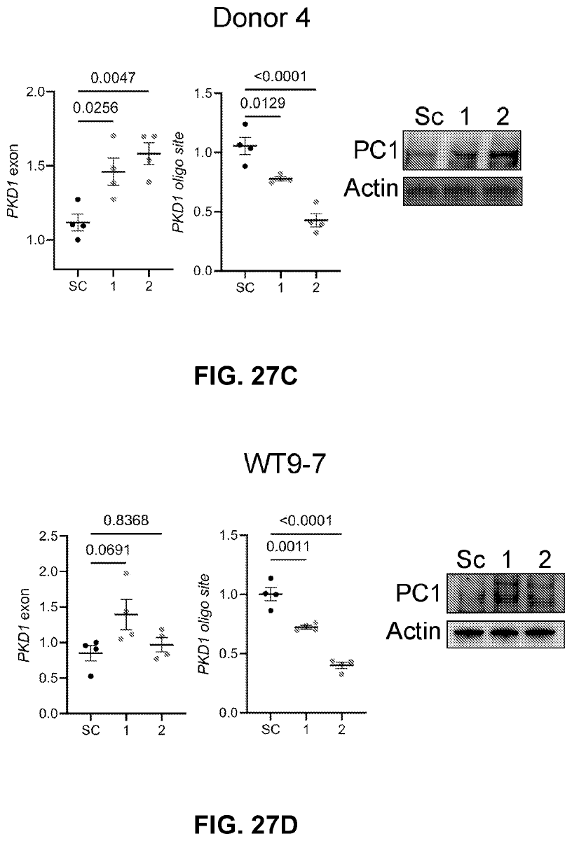

In this example, two Pkd1 oligos are tested in human and mouse ADPKD cell lines and systems. In a first experiment, murine Pkd1^{RC/-} cells were treated with scrambled, Pkd1 oligo #1 (SEQ ID NO: 1) or Pkd1 oligo #2 (SEQ ID NO: 2) and analyzed for levels of Pkd1 mRNA transcript or Pkd1 3′-UTR transcript (containing the binding site for each oligo). Protein expression of Polycystin 1 in treated cells was also measured using western blot. FIG. 27A shows that each Pkd1 oligo binding increases total transcript of Pkd1 mRNA (far left plot) but prevents cDNA synthesis over Pkd1 3-UTR binding site as evidenced by reduced Pkd1 3′-UTR transcript detection in Pkd1 oligo #1 or #2 treated cells (middle plot). The increased transcript correlates to increased protein expression, as evidenced by increased Polycystin levels as measured by Western blot (far right image).

In a second experiment, human ADPKD cells from three donors (Donor 3, Donor 4 and WT9-7) were treated in the same way with scrambled, Pkd1 oligo #1 (SEQ ID NO: 1) or Pkd1 oligo #2 (SEQ ID NO: 2). Increased levels of the Pkd1 mRNA transcript (full), decreased levels of Pkd1 3′-UTR transcript, and increased protein expression of Polycystin-1 (via Western Blot) were found after treatment with each Pkd1 oligo in each cell line (FIG. 27B-27D). Therefore, both Pkd1 oligos (SEQ ID NO: 1 and SEQ ID NO: 2) are effective at increasing PKD1 dosage in both human and mouse cell lines.

### Example 13—Pkd2 Oligo Increases PKD2 Expression in mIMCD3 Cells

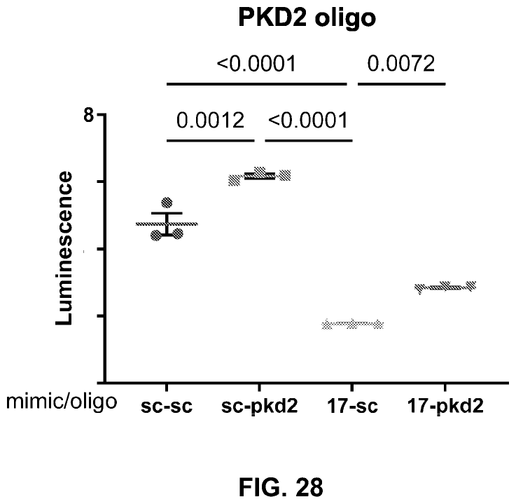

mIMCD3 cells were transfected with pls-PKD2-3′-UTR reporter plasmid, microRNA mimic (sc or miR-17) and scrambled (sc) or Pkd2 oligo (SEQ ID NO: 3). Cells were also transfected with 0.04 μg of the pGL3-Control plasmid (Promega Corp) encoding Photinus luciferase to serve as control for differences in transfection efficiency. Levels of luciferase were measured 72 hours after transfection as a reporter for PKD2 expression (see Methods in Example 14). FIG. 28 shows that cells treated with PKD2 oligo in the presence of the microRNA (miR-17) had increased signal and therefore increased PKD2 expression compared to cells treated with the microRNA alone.

### Example 14—Materials and Methods Used in Examples 9-13 Luciferase Assay

mIMCD3 cells were seeded into six-well dishes (2×10⁵ cells per well) and transfected with 0.4 μg of pLS-Pkd1-3′-UTR plasmid, 10 nM of miR-17 or scramble mimic (Dharmacon) and 40 nM of Scramble or Pkd1 oligo or Pkd2 oligo (Qiagen). Cells were also transfected with 0.04 μg of the pGL3-Control plasmid (Promega Corp) encoding Photinus luciferase to serve as control for differences in transfection efficiency. Lipofectamine 2000 (Invitrogen) was used as a transfection reagent. After forty-eight hours the cells were lysed in 250 μl of passive lysis buffer (Promega Corp), and 40 μl of the cell lysate was added to 96-well plates. Photinus and Renilla luciferase activities were measured by using the Dual-Luciferase Reporter Assay System (Promega Corp) according to the manufacturer's directions.

qRT-PKDR

Total RNA was extracted using Qiagen miRNEASY mini kit. One microgram of RNA was treated with DNase I (Invitrogen) and cDNA was produced with Invitrogen First Strand Superscript III cDNA synthesis kit. Q-PKDR was performed using iQ SYBR Green Supermix (Bio-Rad). All samples were loaded in duplicate or triplicate for analysis on the CFX Connect™ Real-time PKDR. 18s rRNA was used to normalize mRNA expression.

### Western Blot

Cells were scraped with cell scrapers from 6 well plates in 1×PBS and then centrifuged for 5 minutes to isolate pellet. Total protein was extracted from cell pellets using tissue protein extraction reagent (Invitrogen, catalog #78510) with a protease phosphatase inhibitor tablet (Fisher, catalog #PIA32961). 20-50 μg of protein were loaded on NuPAGE™ 3-8% Tris-Acetate Protein Gel (Invitrogen, EA03785) alongside high molecular weight ladder (Invitrogen, catalog #LC5699) at 160 V for 1.5 h on ice. Proteins were transferred onto nitrocellulose membrane using Invitrogen Wet Tank Transfer system at 200 mAmps for 100 minutes on ice or at 4° C. Membrane was blocked for 45 minutes in 5% milk in 1×TBS-Tween and then probed with PKD1 antibody (Santa Cruz 7E12) at 1:500 dilution overnight or Actin-HRP (Sigma) at 1:40,000 for 1 hour. The next morning membrane was washed three times with 1×TBS-tween and then Goat-anti-mouse conjugated HRP was applied as secondary antibody at 1:5000 dilution for one hour. Blots were developed using chemiluminescence substrate (Super-signal West Femto or ECL) reagent from Pierce and visualized on Bio-Rad Digital Imager.

mRNA Stability Assay

Pkd1^{RC/-} cells were plated into 6 well plates (1.5×10⁵ cells per well) and the next morning transfected with Scramble or Pkd1 oligo using Lipofectamine 3000 (Invitrogen) to reach a final concentration of 40 nM. 48 hours after transfection, culture media was replaced with media containing 5 μg/ml media of actinomycin. Cells were harvested at 0, 4 and 8 hours after actinomycin treatment for analysis.

### Cell Lines

Pkd1^{RC/-} cells, kidney epithelial cells and mIMCD3 cells were used in these experiments, mIMCD3 cells were obtained from ATCC. Kidney epithelial cells and Pkd1^{RC/-} cells are immortalized tubule derived kidney epithelial cells derived in our laboratory from 12 day old male mouse kidneys.

### 3D Cystogenesis Assay

8-well chamber slides and 200 μl sterile pipette tips were pre-cooled at −20° C. for a minimum of 6 hours. The floor of each well of an 8-well chamber slide was carefully coated with 25 μl of 100% Matrigel (Fisher, catalog #354234) using 200 μl sterile pipette tips. The plate was then placed in a 37° C. incubator for 30 min to set the Matrigel. During this time. cells were washed with 1×PBS, trypsinized and filtered through a 40 μm cell strainer to create a single-cell suspension. Cells were counted with Hemocytometer and diluted to reach a final concentration of 5000 cells per 150 μl. 150 μl of cell suspension was combined 1:1 with 4% Matrigel and placed in each well. Each treatment condition was seeded in triplicate and incubated at 37° C. for 7 days. After 72 hours, each well was supplemented with 100 μl of epithelial media. On day 7, the cysts were imaged using a Leica DMI 3000B light microscope. The images were analyzed using ImageJ software to measure cyst size. Each assay was repeated three times.

### Immunofluorescence Staining

Cells were fixed with ice-cold 100% methanol for five minutes at 4° C. and then washed thrice with 1× phosphate buffered saline. The cells were then blocked for a minimum of 30 minutes in 1×PBS+10% goat serum+0.1% BSA+0.1 M glycine+0.1% Tween 20 (blocking solution) at room temperature. Cells were probed with PKDREB antibody (Cell Signaling) at 1:400 dilution in blocking solution overnight at 4° C. Secondary antibody was applied at 1:400 dilution for one hour at room temperature. Cells were counterstained with DAPI diluted 1:100,00 in 1×PBS and visualized using Carl Zeiss Compound Light Microscope. All conditions for each experiment were processed and imaged simultaneously.

### Mito Tracker Staining

Cells were washed with 1×PBS and then incubated in MitoTracker Red CMXRos (Thermo Fisher) in serum-free DMEM media at 100 nM concentration for eight minutes. The serum-free media was then replaced with regular epithelial media and cells were immediately imaged using Carl Zeiss Compound Light microscope. All images were taken at the same exposure to compare intensity of MitoTracker fluorescence which is directly proportional to membrane potential.

### RNA Sensor Experiments

Pkd1^{RC/-} cells were plated into 6 well plates (1×10⁵ cells per well) and the next morning transfected with Scramble or Pkd1 oligo and pDAC565 plasmid (Addgene #195242) which bears a guide RNA targeting exon 4 of the Pkd1 gene (CAGCCACGCCAGACCACAGTTGCACTCAAATG; SEQ ID NO: 39) using Lipofectamine 3000 (Invitrogen) to reach a final concentration of 40 nM. After 48 hours cells were visualized under 40× fluorescence microscopy. Quantification of Pkd1 mRNA abundance was determined by counting number of cells which were transfected with plasmid (indicated by background nuclear green fluorescence) that displayed GFP expressing puncta or cloud like signal (indicative for Pkd1 mRNA). 200 cells with GFP glow were counted per each condition. Each experiment was completed 3 times.

1. A method of selectively increasing expression of polycystin 1 and/or polycystin 2 in a cell, the method comprising delivering an antisense oligonucleotide (ASO) that hybridizes to a 3′UTR regulatory region on an mRNA encoding polycystin 1 or polycystin 2 (PKD1 or PKD2 mRNA to the cell.

2. The method of claim 1, wherein the ASO interferes with a microRNA hybridizing to the 3′UTR regulatory region.

3. The method of claim 2, wherein the microRNA is microRNA-17.

4. The method of claim 1, wherein the 3′UTR regulatory region comprises a cis-inhibitory motif.

5. The method of claim 1, wherein the ASO stabilizes the PKD1 and/or PKD2 mRNA.

6. The method of claim 1, wherein the ASO comprises at least 9 nucleotides.

7. The method of claim 6, wherein the ASO comprises 9 to 24 nucleotides.

8. The method of claim 7, wherein the ASO consists of 15 or 16 nucleotides.

9. The method of any one of claims 1 to 8, wherein the ASO hybridizes to at least 6, at least 7, at least 8, at least 9, at least 10, at least 11, at least 12, at least 13, at least 14, at least 15, or at least 16 consecutive nucleotides of a 3′UTR regulatory region on an mRNA encoding polycystin 1 (PKD1 mRNA).

10. The method of claim 9, wherein the 3′UTR regulatory region on an mRNA encoding polycystin 1 (PKD1 mRNA) has a nucleic acid sequence of any one of SEQ ID NOs: 4 to 7.

11. The method of claim 10, wherein the ASO comprises a nucleic acid sequence having at least at least 50%, at least 60%, at least 70% or at least 80%, at least 90%, at least 95%, at least 96%, at least 97%, at least 98%, or at least 99% identity to SEQ ID NO: 1 or SEQ ID NO: 2.

12. The method of claim 11, wherein the ASO comprises a nucleic acid sequence comprising or consisting of SEQ ID NO: 1 or SEQ ID NO: 2.

13. The method of any one of claims 1 to 8, wherein the ASO hybridizes to at least 6, at least 7, at least 8, at least 9, at least 10, at least 11, at least 12, at least 13, at least 14, at least 15, or at least 16 consecutive nucleotides of a 3′UTR regulatory region on an mRNA encoding polycystin 2 (PKD2 mRNA).

14. The method of claim 13, wherein the 3′UTR regulatory region on an mRNA encoding polycystin 1 (PKD2 mRNA) has a nucleic acid sequence of any one of SEQ ID NOs: 8 to 13.

15. The method of claim 14, wherein the ASO comprises a nucleic acid sequence having at least at least 50%, at least 60%, at least 70% or at least 80%, at least 90%, at least 95%, at least 96%, at least 97%, at least 98%, or at least 99% identity to SEQ ID NO: 3.

16. The method of claim 15, wherein the ASO comprises a nucleic acid sequence comprising or consisting of SEQ ID NO: 3.

17. The method of any one of claims 1 to 16, wherein the cell carries a mutation in at least one allele of the PKD1 and/or PKD2 gene and has reduced baseline expression of polycystin 1 and/or polycystin 2 compared to a cell without the mutation.

18. The method of claim 17, wherein expression of polycystin 1 and/or polycystin 2 is increased by at least 25%, at least 30%, at least 40%, at least 50%, at least 60%, at least 70% or at least 80% over baseline.

19. The method of any one of claims 1 to 18, wherein the cell is in vitro.

20. The method of claims 1 to 19, wherein the cell is in vivo.

21. The method of any one of claims 1 to 20, wherein the cell is human or murine.

22. A method of treating Autosomal Dominant Polycystic Kidney Disease (ADPKD) in a subject in need thereof, the method comprising administering a pharmaceutically effective amount of an antisense oligonucleotide (ASO) that hybridizes to a 3′UTR regulatory region on an mRNA encoding polycystin 1 or polycystin 2 (PKD1 or PKD2 mRNA to the subject.

23. The method of claim 22, wherein the ASO interferes with a microRNA hybridizing to the 3′UTR regulatory region.

24. The method of claim 23, wherein the microRNA is microRNA-17.

25. The method of claim 22, wherein the 3′UTR regulatory region comprises a cis-inhibitory motif.

26. The method of claim 22, wherein the ASO stabilizes the PKD1 and/or PKD2 mRNA.

27. The method of claim 22, wherein the ASO comprises at least 9 nucleotides.

28. The method of claim 27, wherein the ASO comprises 9 to 24 nucleotides.

29. The method of claim 28, wherein the ASO consists of 15 or 16 nucleotides.

30. The method of any one of claims 22 to 29, wherein the ASO hybridizes to at least 6, at least 7, at least 8, at least 9, at least 10, at least 11, at least 12, at least 13, at least 14, at least 15, or at least 16 consecutive nucleotides of a 3′UTR regulatory region on an mRNA encoding polycystin 1 (PKD1 mRNA).

31. The method of claim 30, wherein the 3′UTR regulatory region on an mRNA encoding polycystin 1 (PKD1 mRNA) has a nucleic acid sequence of any one of SEQ ID NOs: 4 to 7.

32. The method of claim 31, wherein the ASO comprises a nucleic acid sequence having at least at least 50%, at least 60%, at least 70% or at least 80%, at least 90%, at least 95%, at least 96%, at least 97%, at least 98%, or at least 99% identity to SEQ ID NO: 1 or SEQ ID NO: 2.

33. The method of claim 32, wherein the ASO comprises a nucleic acid sequence comprising or consisting of SEQ ID NO: 1 or SEQ ID NO: 2

34. The method of any one of claims 22 to 29, wherein the ASO hybridizes to at least 6, at least 7, at least 8, at least 9, at least 10, at least 11, at least 12, at least 13, at least 14, at least 15, or at least 16 consecutive nucleotides of a 3′UTR regulatory region on an mRNA encoding polycystin 2 (PKD2 mRNA).

35. The method of claim 34, wherein the 3′UTR regulatory region on an mRNA encoding polycystin 1 (PKD2 mRNA) has a nucleic acid sequence of any one of SEQ ID NOs: 8 to 13.

36. The method of claim 35, wherein the ASO comprises a nucleic acid sequence having at least at least 50%, at least 60%, at least 70% or at least 80%, at least 90%, at least 95%, at least 96%, at least 97%, at least 98%, or at least 99% identity to SEQ ID NO: 3.

37. The method of claim 36, wherein the ASO comprises a nucleic acid sequence comprising or consisting of SEQ ID NO: 3.

38. The method of any one of claims 22 to 37, wherein the ASO is administered systemically.

39. The method of claim 38, wherein the ASO is administered orally, intravenously, subcutaneously, or intraperitoneally.

40. The method of any one of claims 22 to 39, wherein the ASO is administered as a pharmaceutical composition.

41. The method of any one of claims 22 to 40, wherein the subject is human.

42. An antisense oligonucleotide (ASO) that hybridizes to a 3′UTR regulatory region on an mRNA encoding polycystin 1 or polycystin 2 (PKD1 or PKD2 mRNA).

43. The ASO of claim 42, wherein the ASO hybridizes to at least 6, at least 7, at least 8, at least 9, at least 10, at least 11, at least 12, at least 13, at least 14, at least 15, or at least 16 consecutive nucleotides of a 3′UTR regulatory region on an mRNA encoding polycystin 1 (PKD1 mRNA).

44. The ASO of claim 43, wherein the 3′UTR regulatory region on an mRNA encoding polycystin 1 (PKD1 mRNA) comprises a nucleic acid sequence of any one of SEQ ID NOs: 4 to 7.

45. The ASO of claim 44, wherein the ASO comprises a nucleic acid sequence having at least at least 50%, at least 60%, at least 70% or at least 80%, at least 90%, at least 95%, at least 96%, at least 97%, at least 98%, or at least 99% identity to SEQ ID NO: 1 or SEQ ID NO: 2.

46. The ASO of claim 45, wherein the ASO comprises a nucleic acid sequence comprising or consisting of SEQ ID NO: 1 or SEQ ID NO: 2.

47. The ASO of claim 42, wherein the ASO hybridizes to at least 6, at least 7, at least 8, at least 9, at least 10, at least 11, at least 12, at least 13, at least 14, at least 15, or at least 16 consecutive nucleotides of a 3′UTR regulatory region on an mRNA encoding polycystin 2 (PKD2 mRNA).

48. The ASO of claim 47, wherein the 3′UTR regulatory region on an mRNA encoding polycystin 1 (PKD2 mRNA) has a nucleic acid sequence of any one of SEQ ID NOs: 8 to 13.

49. The ASO of claim 48, wherein ASO comprises a nucleic acid sequence having at least at least 50%, at least 60%, at least 70% or at least 80%, at least 90%, at least 95%, at least 96%, at least 97%, at least 98%, or at least 99% identity to SEQ ID NO: 3.

50. The ASO of claim 49, wherein the ASO comprises a nucleic acid sequence comprising or consisting of SEQ ID NO: 3.

51. The ASO of any one of claims 42 to 50, further comprising at least one locked nucleic acid.

52. A pharmaceutical composition comprising an ASO of claim 51 and at least one carrier or excipient.

53. A kit comprising the pharmaceutical composition of claim 52.
